(19) United States
(12) Patent Application Publication
(10) Pub. No.: US 2025/0084372 A1
(43) Pub. Date: Mar. 13, 2025

(54) IN VITRO DERIVATION OF GONADAL SOMATIC CELLS

(71) Applicant: Conception Bisosciences, Inc., Berkeley, CA (US)

(72) Inventors: Rhishikesh BARGAJE, BERKELEY, CA (US); Karmen Seres, Berkeley, CA (US); Pablo Hurtado-Gonzalez, Berkeley, CA (US); Alyssa Miller, Berkeley, CA (US)

(73) Assignee: Conception Biosciences, Inc., Berkeley, CA (US)

(21) Appl. No.: 18/310,161

(22) PCT Filed: Nov. 1, 2021
(86) PCT No.: PCT/US2021/072165

§ 371 Date: May 1, 2023

Related U.S. Application Data
(60) Provisional application No. 63/222,953, filed on Jul. 16, 2021.
(60) Provisional application No. 63/108,666, filed on Nov. 2, 2020.

(51) Int. Cl. C12N 5/071

(52) U.S. Cl. CPC C12N 5/0608; C12N 2501/115; C12N 2501/119; C12N 2501/155; C12N 2501/727; C12N 2506/45; C12N 2533/52

(57) ABSTRACT

Provided herein are methods of generating gonadal cell populations such as ovarian somatic cells from pluripotent stem cells. Also included are gonadal cell populations, as well as intermediate cell populations generated therein.

### CROSS-REFERENCE TO RELATED APPLICATIONS

This application claims priority to U.S. provisional applications 63/108,666, filed Nov. 2, 2020, entitled “HUMAN GRANULOSA DIFFERENTIATION”; and 63/222,953, filed Jul. 16, 2021, entitled “IN VITRO DERIVATION OF GONADAL SOMATIC CELLS” the contents of which are incorporated by reference in their entirety for all purposes.

### FIELD OF THE INVENTION

The present disclosure relates generally to methods of generating gonadal cell populations such as ovarian somatic cells from pluripotent stem cells. Also included are gonadal cell populations, as well as intermediate cell populations generated therein.

### BRIEF SUMMARY OF THE INVENTION

Provided herein is a method of producing a gonadal cell population, the method comprising: (a) culturing pluripotent stem cells in a mesoderm induction medium comprising Activin A for a first period of time to produce a first intermediate cell population; (b) culturing the first intermediate cell population in an intermediate mesoderm induction medium comprising an retinoic acid pathway modulator (RAPM) for a second period of time, thereby producing a second intermediate cell population; and (c) culturing the second intermediate cell population in a gonadal induction medium comprising BMP and FGF, for a third period of time to produce the gonadal cell population. In some embodiments, provided herein is a method of producing a gonadal cell population, the method comprising: (a) culturing mesoderm or mesoderm-like cells in an intermediate mesoderm induction medium comprising a retinoic acid pathway modulator (RAPM) for a first period of time thereby producing a second intermediate cell population; and (b) culturing the second intermediate cell population in a gonadal induction medium comprising BMP, and FGF for a second period of time to produce the gonadal cell population. In some embodiments, there is provided a method of producing a gonadal cell population, the method comprising: culturing intermediate mesoderm or intermediate mesoderm-like cells in a gonadal induction medium comprising, BMP and FGF for a period of time to produce the gonadal cell population. In some embodiments, the BMP is a BMP4, BMP2, BMP7, BMP15, or any combination thereof. In some embodiments, the gonadal induction medium includes BMP4. In some embodiments, the gonadal induction medium further comprises follistatin.

Provided herein is a method of producing a gonadal cell population, the method comprising: (a) culturing pluripotent stem cells in a mesoderm induction medium comprising Activin A for a first period of time to produce a first intermediate cell population; (b) culturing the first intermediate cell population in an intermediate mesoderm induction medium comprising an retinoic acid pathway modulator (RAPM) for a second period of time, thereby producing a second intermediate cell population; and (c) culturing the second intermediate cell population in a gonadal induction medium comprising follistatin, BMP4 and FGF, for a third period of time to produce the gonadal cell population.

Also provided herein is a method of producing a gonadal cell population, the method comprising: (a) culturing mesoderm or mesoderm-like cells in an intermediate mesoderm induction medium comprising a retinoic acid pathway modulator (RAPM) for a first period of time thereby producing a second intermediate cell population; and (b) culturing the second intermediate cell population in a gonadal induction medium comprising follistatin, BMP4, and FGF for a second period of time to produce the gonadal cell population.

Also provided herein is a method of producing a gonadal cell population, the method comprising culturing intermediate mesoderm or intermediate mesoderm-like cells in a gonadal induction medium comprising follistatin, BMP4 and FGF for a period of time to produce the gonadal cell population.

Also provided herein is a method of producing a first intermediate cell population, the method comprising: culturing pluripotent stem cells in a mesoderm induction medium comprising Activin A and a glycogen synthase kinase-3 inhibitor for a period of time to produce a first intermediate cell population Also provided herein is a method of producing a second intermediate cell population, the method comprising culturing mesoderm or mesoderm-like cells in an intermediate mesoderm induction medium comprising a retinoic acid pathway modulator (RAPM) for a period of time, thereby producing the second intermediate cell population. Also provided herein is a method of producing a second intermediate cell population, the method comprising: (a) culturing pluripotent stem cells in a mesoderm induction medium comprising activin A and a glycogen synthase kinase-3 inhibitor for a first period of time to produce a first intermediate cell population; and (b) culturing the first intermediate cell population in an intermediate mesoderm induction medium comprising a retinoic acid pathway modulator (RAPM), FGF, and a glycogen synthase kinase-3 inhibitor for a second period of time, thereby producing the second intermediate cell population. In some of any embodiments, the first intermediate cell population and second intermediate cell population are intermediate populations of differentiated pluripotent stem cells for producing a gonadal cell population.

In some of any embodiments, at least a portion of cells in the first intermediate cell population express Brachyury.

In some of any embodiments, at least a portion of the cells in the second intermediate cell population express OSR1, PAX2, or LHX.

In some of any embodiments, at least a portion of the cells in the gonadal cell population express FOXL2, NR2F2, or RUNX1.

In some of any embodiments, the pluripotent stem cells are seeded at a density of about 10.000 to about 40,000 cells per cm2. In some of any embodiments, the pluripotent stem cells are seeded in a culture plate coated with fibronectin. In some of any embodiments, the pluroipotent stem cells are seeded in a culture plate coated with matrigel.

In some of any embodiments, the mesoderm induction medium further comprises FGF. In some of any embodiments, the mesoderm induction medium further comprises BMP4. In some of any embodiments, the mesoderm induction medium further comprises a glycogen synthase kinase-3 inhibitor. In some of any embodiments, the mesoderm induction medium further comprises an apoptosis inhibitor.

In some of any embodiments, the concentration of Activin A in the mesoderm induction medium is about 30 ng/ml to about 70 ng/mL. In some of any embodiments, the concentration of Activin A in the mesoderm induction medium is about 50 ng/ml.

In some of any embodiments, the FGF in the mesoderm induction medium is FGF2. In some embodiments, the concentration of FGF2 in the mesoderm induction medium is about 5 ng/mL to about 20 ng/mL. In some embodiments, the concentration of FGF2 in the mesoderm induction medium is about 12 ng/ml.

In some of any embodiments, the concentration of BMP4 in the mesoderm induction medium is about 10 ng/mL to about 50 ng/mL. In some embodiments, the concentration of BMP4 in the mesoderm induction medium is about 30 ng/mL.

In some of any embodiments, the glycogen synthase kinase-3 inhibitor in the mesoderm induction medium is CHIR99021. In some embodiments, the concentration of CHIR99021 is about 1 μM to about 5 μM. In some embodiments, the concentration of CHIR99021 in the mesoderm induction medium is about 3 μM.

In some of any embodiments, within the mesoderm-induction medium the apoptosis inhibitor is Y-27632. In some embodiments, the concentration of Y-27632 is about 5 μM to about 20 μM. In some embodiments, the concentration of Y-27632 is about 10 μM. In some of any embodiments, within the mesoderm-induction medium the apoptosis inhibitor comprises Chroman1, Emricasan, and Trans-ISRIB. In some embodiments, the apoptosis inhibitor is Chroman1 and the concentration of Chroman1 is about 30 nM to about 70 nM. In some embodiments, the concentration of Chroman1 is about 50 nM. In some embodiments, the apoptosis inhibitor is Emricasan and the concentration of Emricasan is about 2 μM to about 10 μM. In some embodiments, the concentration of Emricasan is about 5 μM. In some embodiments, the apoptosis inhibitor is Trans-ISRIB and the concentration of Trans-ISRIB is about 0.2 μM to about 2 μM. In some embodiments, the concentration of Trans-ISRIB is about 0.7 μM.

In some of any embodiments, the period of time for culturing in mesoderm induction medium is about 24 hours to about 96 hours. In some of any embodiments, the period of time for culturing in mesoderm induction medium is about 24 hours to about 72 hours. In some of any embodiments, the period of time for culturing in mesoderm induction medium is about 56 hours to about 72 hours.

In some of any embodiments, at least 90% of cells in the first intermediate cell population express Brachyury. In some of any embodiments, at least 80% of cells in the first intermediate cell population express one or more of: MIXL1, N-Cadherin, EpCam, NCAM, In some of any embodiments, at least 90% of cells in the first intermediate cell population expresses Brachyury, N-Cadherin, EpCam, and NCAM. In some of any embodiments, at least 90% of cells in the first intermediate cell population are mesoderm or mesoderm-like cells. In some of any embodiments, the first intermediate cell population consists essentially of mesoderm or mesoderm-like cells.

In some of any embodiments, the first intermediate cell population is replated onto a new fibronectin-coated culture plate prior to culturing in intermediate mesoderm induction medium. In some embodiments, the first intermediate cell population is enzymatically detached, centrifuged and resuspended before replating. In some of any embodiments, the first intermediate cell population is plated a density of about 5000 to about 25000 cells/cm2.

In some of any embodiments, the intermediate mesoderm induction medium further comprises an FGF. In some of any embodiments, the intermediate mesoderm induction medium further comprises a glycogen synthase kinase-3 inhibitor. In some of any embodiments, the intermediate mesoderm induction medium further comprises Activin A. In some of any embodiments, the intermediate mesoderm induction medium further comprises an apoptosis inhibitor.

In some of any embodiments, the method comprises culturing the first intermediate cell population (i) first in the intermediate mesoderm induction medium with a first concentration of the apoptosis inhibitor. (ii) subsequently in the intermediate mesoderm induction medium with no more than a second concentration of the apoptosis inhibitor.

In some of any embodiments, the RAPM in the intermediate mesoderm induction medium is an RAR agonist. In some embodiments, the RAPM comprises retionic acid (RA) and/or TTNPB. In some embodiments, the RAPM is RA. In some embodiments, the concentration of RA in the intermediate mesoderm induction medium is about 0.5 μM to about 2 μM. In some embodiments, the concentration of RA in the intermediate mesoderm induction medium is about 1 μM. In some embodiments, the RAPM is TTNPB. In some embodiments, the concentration of TTNPB in the intermediate mesoderm induction medium is about 0.2 μM to about 1 μM. In some embodiments, the concentration of TTNPB in the intermediate mesoderm induction medium is about 0.5 μM.

In some of any embodiments, the FGF in the intermediate mesoderm induction medium is FGF2. In some embodiments, the concentration of the FGF2 is about 10 ng/mL to about 30 ng/mL. In some embodiments, the concentration of the FGF2 in the intermediate mesoderm induction medium is about 20 ng/ml.

In some of any embodiments, the glycogen synthase kinase-3 inhibitor in the intermediate mesoderm induction medium is CHIR99021. In some embodiments, the concentration of CHIR99021 is about 1 μM to about 5 μM. In some embodiments, the concentration of CHIR99021 in the intermediate mesoderm induction medium is about 3 μM.

In some of any embodiments, the concentration of Activin A in the intermediate mesoderm induction medium is about 10 ng/ml to about 50 ng/mL. In some embodiments, the concentration of Activin A in the intermediate mesoderm induction medium is about 30 ng/mL.

In some of any embodiments, within the intermediate mesoderm-induction medium the apoptosis inhibitor is Y-27632. In some embodiments, the concentration of Y-27632 is about 5 μM to about 20 μM. In some embodiments, within the intermediate mesoderm-induction medium, the apoptosis inhibitor comprises Chroman1, Emricasan, and Trans-ISRIB. In some embodiments, the apoptosis inhibitor is Chroman1. In some embodiments, the concentration of Chroman1 is about 30 nM to about 70 nM. In some embodiments, the apoptosis inhibitor is Emricasan. In some embodiments, the concentration of Emricasan is about 2 μM to about 10 μM. In some embodiments, the apoptosis inhibitor is Trans-ISRIB. In some embodiments, the concentration of Trans-ISRIB is about 0.2 μM to about 2 μM.

In some embodiments, the apoptosis inhibitor in the intermediate mesoderm induction medium is Y-27632, wherein the method comprises culturing the first intermediate cell population (i) first in the intermediate mesoderm induction medium comprising about 10 μM of Y-27632, and (ii) subsequently in the intermediate mesoderm induction medium with no more than about 2 μM of Y-27632. In some embodiments, the method comprises culturing the first intermediate cell population: (i) first in the intermediate mesoderm induction medium comprising about 10 μM of Y-27632 for about 24 hours, and (ii) subsequently in the intermediate mesoderm induction medium with no more than about 2 μM of Y-27632 for about 5-6 days.

In some of any embodiments, the period of time for culturing in the intermediate mesoderm induction medium is about 4-14 days. In some of any embodiments, the period of time for culturing in the intermediate mesoderm induction medium is about 5-9 days.

In some of any embodiments, the apoptosis inhibitor in the intermediate mesoderm induction medium is Y-27632, wherein the concentration of Y-27632 is: (a) about 10 μM during the first 24 hours; (b) no more than about 2 μM between 24 hours and 72 hours; (c) no more than about 0.5 μM between 72 hours and 120 hours; or (d) no more than about 0.1 μM after 120 hours, each during the culturing in the intermediate mesoderm induction medium. In some of any embodiments, the first intermediate cell population is cultured in intermediate mesoderm induction medium comprising an apoptosis inhibitor for about 24 hours, and wherein after about 24 hours, a portion of the medium is first replaced with an intermediate mesoderm induction medium not comprising the apoptosis inhibitor. In some embodiments, the portion of the medium is about 80% of the medium. In some embodiments, the method further comprises subsequent medium replacements, wherein the medium replacements comprise replacing a portion of the medium every about 48 hours after the initial 24 hours of culturing. In some embodiments, the portion of the medium in each subsequent medium replacement is about 80% of the medium.

In some of any embodiments, at least 90% of cells in the second intermediate cell population expresses one or more of: OSR1, PAX2, LHX1, and RUNX1. In some of any embodiments, at least 90% of cells in the second intermediate cell population expresses two or more of: OSR1, PAX2, LHX1, and RUNX1. In some of any embodiments, at least 90% of the cells in the second intermediate cell population expresses OSR1, PAX2, and LHX1. In some of any embodiments, at least 90% of cells in the second intermediate cell population are intermediate mesoderm or intermediate mesoderm-like cells. In some of any embodiments, the second intermediate cell population consists essentially of intermediate mesoderm or intermediate mesoderm-like cells.

In some of any embodiments, the second intermediate cell population is replated onto a new fibronectin-coated culture plate prior to culturing in gonadal induction medium. In some embodiments, the second intermediate cell population is enzymatically detached, centrifuged and resuspended before replating. In some embodiments, the second intermediate cell population is plated at a density of about 5000 to about 25000 cells/cm2.

In some of any embodiments, the gonadal induction medium further comprises an RAPM. In some embodiments, the RAPM in the gonadal induction medium is an RAR agonist. In some embodiments, the RAPM comprises retionic acid (RA) and/or TTNPB. In some embodiments, the RAPM is RA. In some embodiments, the concentration of RA in the gonadal induction medium is about 0.5 μM to about 2 μM. In some embodiments, the RAPM is TTNPB. In some embodiments, the concentration of TTNPB in the gonadal induction medium is about 0.2 μM to about 1 μM.

In some of any embodiments, the gonadal induction medium further comprises an apoptosis inhibitor. In some embodiments, within the gonadal induction medium the apoptosis inhibitor is Y-27632. In some embodiments, the concentration of Y-27632 is about 5 μM to about 20 μM. In some embodiments, the concentration of Y-27632 is about 10 μM. In some embodiments, within the gonadal induction medium, the apoptosis inhibitor comprises Chroman1, Emricasan, and Trans-ISRIB. In some embodiments, the apoptosis inhibitor is Chroman1 and the concentration of Chroman1 is about 30 nM to about 70 nM. In some embodiments, the concentration of Chroman 1 is about 50 nM. In some embodiments, the apoptosis inhibitor is Emricasan and the concentration of Emricasan is about 2 μM to about 10 μM. In some embodiments, the concentration of Emricasan is about 5 μM. In some embodiments, the apoptosis inhibitor is Trans-ISRIB and the concentration of Trans-ISRIB is about 0.2 μM to about 2 μM. In some embodiments, the concentration of Trans-ISRIB is about 0.7 μM.

In some of any embodiments, the gonadal induction medium further comprises follistatin.

In some of any embodiments, the concentration of follistatin in the gonadal induction medium is about 10 ng/mL to about 50 ng/mL. In some embodiments, the concentration of follistatin in the gonadal induction medium about 25 ng/mL.

In some of any embodiments, the BMP in the gonadal induction medium comprises BMP4, BMP2. BMP7. BMP15, or any combinations thereof. In some embodiments, the gonadal induction medium includes at least BMP4, alone or in combination with another BMP. In some embodiments, the only BMP included in the gonadal induction medium is BMP4. In some embodiments, the total concentration of BMP in the gonadal induction medium is about 5 ng/ml to about 20 ng/ml, or about 20 ng/ml to about 70 ng/mL. In some embodiments, the total concentration of BMP in the gonadal induction medium is about 10 ng/ml or about 50 ng/mL.

In some of any embodiments, the concentration of BMP4 in the gonadal induction medium is about 5 ng/ml to about 20 ng/ml. In some of any embodiments, the concentration of BMP4 in the gonadal induction medium or about 20 ng/ml to about 70 ng/mL. In some of any embodiments, the concentration of BMP4 in the gonadal induction medium is about 10 ng/ml or about 50 ng/mL.

In some of any embodiments, the FGF in the gonadal induction medium comprises FGF2, FGF9, FGF10, FGF16, FGF17, FGF18, FGF19, or any combinations thereof. In some embodiments, the gonadal induction medium includes at least FGF2, alone or in combination with another FGF. In some embodiments, the only FGF included in the gonadal induction medium is FGF2. In some embodiments, the concentration of FGF is about 1 ng/ml to about 10 ng/mL or about 5 ng/ml to about 25 ng/ml. In some embodiments, the concentration of FGF is about 5 ng/ml or about 10 ng/mL.

In some of any embodiments, the FGF in the gonadal induction medium is FGF2. In some embodiments, the concentration of the FGF2 is about 1 ng/ml to about 10 ng/mL. In some embodiments, the concentration of the FGF2 is about 5 ng/ml to about 25 ng/mL. In soe embodiments, the concentration of the FGF2 in the gonadal induction medium is about 5 ng/ml or about 10 ng/mL.

In some of any embodiments, the period of time for culturing in gonadal induction medium is about 5 days to about 21 days. In some of any embodiments, the period of time for culturing in gonadal induction medium is about 7 days to about 14 days.

In some of any embodiments, the gonadal cell population comprises ovarian somatic cells. In some of any embodiments, he gonadal cell population consists of ovarian somatic cells.

In some of any embodiments, at least 20% of cells within the gonadal cell population are FOXL2-positive cells. In some of any embodiments, at least 20% of the cells within the gonadal cell population are NR2F2-positive cells. In some of any embodiments, at least 20% of the cells within the gonadal cell population are KRT19-positive cells. In some of any embodiments, at least 90% of the gonadal cell population are FOXL2-positive cells, NR2F2-positive cells, and/or KRT-19 positive cells. In some of any embodiments, the gonadal cell population consists essentially of FOXL2-positive cells, NR2F2-positive cells, and/or KRT-19 positive cells. In some of any embodiments, the FOXL2-positive cells comprise granulosa cells. In some of any embodiments, the NR2F2-positive cells comprise stroma cells and/or granulosa cells. In some of any embodiments, the KRT-19 positive cells comprise ovarian epithelial cells.

In some of any embodiments, in the gonadal cell population generated by the method, as compared to a corresponding gonadal cell population generated by a method wherein the intermediate mesoderm induction medium does not comprise RAPM, one or more of: (I) the amount of NR2F2-expressing cells in the gonadal cell population is increased; and/or (II) the amount of FOXL2-expressing cells in the gonadal cell population is increased; and/or (III) the amount of RUNX1-expressing cells in the gonadal cell population is increased; and/or (IV) the amount of WNT6-expressing cells in the gonadal cell population is increased; and/or (V) the amount of NR5A1-expressing cells in the gonadal cell population is increased; and/or (VI) the amount of OSR1-expressing cells in the gonadal cell population is increased; and/or (VII) the amount of LHX9-expressing cells in the gonadal cell population is increased; and/or (VIII) the amount of EMX2-expressing cells in the gonadal cell population is increased.

In some of any embodiments, in the gonadal cell population generated by the method, as compared to a corresponding gonadal cell population generated by a method wherein the intermediate mesoderm induction medium comprises a lower concentration of RAPM, one or more of: (I) the amount of NR2F2-expressing cells in the gonadal cell population is increased; and/or (II) the amount of FOXL2-expressing cells in the gonadal cell population is increased; and/or (III) the amount of RUNX1-expressing cells in the gonadal cell population is increased; and/or (IV) the amount of WNT6-expressing cells in the gonadal cell population is increased; and/or (V) the amount of NR5A1-expressing cells in the gonadal cell population is increased; and/or (VI) the amount of OSR1-expressing cells in the gonadal cell population is increased.

In some of any embodiments, in the gonadal cell population generated by the method, as compared to a corresponding gonadal cell population generated by a method wherein the intermediate mesoderm induction medium does not comprise glycogen synthase kinase-3 inhibitor, one or more of: (I) the amount of NR2F2-expressing cells in the gonadal cell population is increased; and/or (II) the amount of FOXL2-expressing cells in the gonadal cell population is increased; and/or (III) the amount of RUNX1-expressing cells in the gonadal cell population is increased; and/or (IV) the amount of WNT6-expressing cells in the gonadal cell population is increased; and/or (V) the amount of NR5A1-expressing cells in the gonadal cell population is increased; and/or (VI) the amount of OSR1-expressing cells in the gonadal cell population is increased; and/or (VII) the amount of LHX9-expressing cells in the gonadal cell population is increased; and/or (VIII) the amount of EMX2-expressing cells in the gonadal cell population is increased.

In some of any embodiments, in the gonadal cell population generated by the method, as compared to a corresponding gonadal cell population generated by a method wherein the intermediate mesoderm induction medium comprises a lower concentration of glycogen synthase kinase-3 inhibitor, one or more of: (I) the amount of NR2F2-expressing cells in the gonadal cell population is increased; and/or (II) the amount of FOXL2-expressing cells in the gonadal cell population is increased; and/or (III) the amount of RUNX1-expressing cells in the gonadal cell population is increased; and/or (IV) the amount of WNT6-expressing cells in the gonadal cell population is increased; and/or (V) the amount of NR5A1-expressing cells in the gonadal cell population is increased; and/or (VI) the amount of OSR1-expressing cells in the gonadal cell population is increased; and/or (VII) the amount of LHX9-expressing cells in the gonadal cell population is increased; and/or (VIII) the amount of EMX2-expressing cells in the gonadal cell population is increased.

In some of any embodiments, in the second intermediate cell population produced by the method, as compared to a corresponding second intermediate cell population generated by a method wherein the intermediate mesoderm induction medium does not comprise RAPM, one or more of: (I) the amount of LHX1 expression in the second intermediate cell population is increased; and/or (II) the amount of PAX2 expression in the second intermediate cell population is increased; and/or (III) the amount of WT1 expression in the second intermediate population is increased; and/or (IV) the amount of RUNX1 expression in the second intermediate cell population is increased; and/or (V) the viability of the second intermediate cell population is increased; and/or (VI) the cell morphology of the second intermediate cell population is more uniform.

In some of any embodiments, in the second intermediate cell population produced by the method, as compared to a corresponding second intermediate cell population generated by a method wherein the intermediate mesoderm induction medium comprises a lower concentration of RAPM, one or more of: (I) the amount of LHX1 expression in the second intermediate cell population is increased; and/or (II) the amount of PAX2 expression in the second intermediate cell population is increased; and/or (III) the amount of WT1 expression in the second intermediate population is increased; and/or (IV) the amount of RUNX1 expression in the second intermediate cell population is increased; and/or (V) the viability of the second intermediate cell population is increased; and/or (VI) the cell morphology of the second intermediate cell population is more uniform.

In some of any embodiments, in the second intermediate cell population produced by the method, as compared to a corresponding second intermediate cell population generated by a method wherein the intermediate mesoderm induction medium docs not comprise glycogen synthase kinase-3 inhibitor, one or more of: (1) the amount of LHX1 expression in the second intermediate cell population is increased; and/or (II) the amount of PAX2 expression in the second intermediate cell population is increased; and/or (III) the amount of WT1 expression in the second intermediate population is increased; and/or (IV) the amount of RUNX1 expression in the second intermediate cell population is increased; and/or (V) the viability of the second intermediate cell population is increased; and/or (VI) the cell morphology of the second intermediate cell population is more uniform.

In some of any embodiments, in the second intermediate cell population produced by the method, as compared to a corresponding second intermediate cell population generated by a method wherein the intermediate mesoderm induction medium comprises a lower concentration of glycogen synthase kinase-3 inhibitor, (I) the amount of LHX1 expression in the second intermediate cell population is increased; and/or (II) the amount of PAX2 expression in the second intermediate cell population is increased; and/or (III) the amount of WT1 expression in the second intermediate population is increased; and/or (IV) the amount of RUNX1 expression in the second intermediate cell population is increased; and/or (V) the viability of the second intermediate cell population is increased; and/or (VI) the cell morphology of the second intermediate cell population is more uniform.

In some of any embodiments, in the second intermediate cell population produced by the method, as compared to a corresponding second intermediate cell population generated by a method wherein the intermediate mesoderm induction medium does not comprise RAPM, one of more of: (I) the potential of the second intermediate cell population to differentiate into NR2F2-expressing gonadal cells is increased; and/or (II) the potential of the second intermediate cell population to differentiate into FOXL2-expressing gonadal cells is increased; and/or (III) the potential of the second intermediate cell population to differentiate into RUNX1-expressing gonadal cells is increased; and/or (IV) the potential of the second intermediate cell population to differentiate into WNT6-expressing gonadal cells is increased; and/or (V) the potential of the second intermediate cell population to differentiate into NR5A1-expressing gonadal cells is increased; and/or (VI) the potential of the second intermediate cell population to differentiate into OSR1-expressing gonadal cells is increased.

In some of any embodiments, in the second intermediate cell population produced by the method, as compared to a corresponding second intermediate cell population generated by a method wherein the intermediate mesoderm induction medium comprises a lower concentration of RAPM. (1) the potential of the second intermediate cell population to differentiate into NR2F2-expressing gonadal cells is increased; and/or (II) the potential of the second intermediate cell population to differentiate into FOXL2-expressing gonadal cells is increased; and/or (III) the potential of the second intermediate cell population to differentiate into RUNX1-expressing gonadal cells is increased; and/or (IV) the potential of the second intermediate cell population to differentiate into WNT6-expressing gonadal cells is increased; and/or (V) the potential of the second intermediate cell population to differentiate into NR5A1-expressing gonadal cells is increased; and/or (VI) the potential of the second intermediate cell population to differentiate into OSR1-expressing gonadal cells is increased.

In some of any embodiments, in the second intermediate cell population produced by the method, as compared to a corresponding second intermediate cell population generated by a method wherein the intermediate mesoderm induction medium does not comprise glycogen synthase kinase-3 inhibitor, one or more of: (I) the potential of the second intermediate cell population to differentiate into NR2F2-expressing gonadal cells is increased; and/or (II) the potential of the second intermediate cell population to differentiate into FOXL2-expressing gonadal cells is increased; and/or (III) the potential of the second intermediate cell population to differentiate into RUNX1-expressing gonadal cells is increased; and/or (IV) the potential of the second intermediate cell population to differentiate into WNT6-expressing gonadal cells is increased; and/or (V) the potential of the second intermediate cell population to differentiate into NR5A1-expressing gonadal cells is increased; and/or (VI) the potential of the second intermediate cell population to differentiate into OSR1-expressing gonadal cells is increased

In some of any embodiments, in the second intermediate cell population produced by the method, as compared to a corresponding second intermediate cell population generated by a method wherein the intermediate mesoderm induction medium comprises a lower concentration of glycogen synthase kinase-3 inhibitor, one or more of: (I) the potential of the second intermediate cell population to differentiate into NR2F2-expressing gonadal cells is increased; and/or (II) the potential of the second intermediate cell population to differentiate into FOXL2-expressing gonadal cells is increased; and/or (III) the potential of the second intermediate cell population to differentiate into RUNX1-expressing gonadal cells is increased; and/or (IV) the potential of the second intermediate cell population to differentiate into WNT6-expressing gonadal cells is increased; and/or (V) the potential of the second intermediate cell population to differentiate into NR5A1-expressing gonadal cells is increased; and/or (VI) the potential of the second intermediate cell population to differentiate into OSR1-expressing gonadal cells is increased.

In some of any embodiments, the gonadal cell population produced by the method is one in which: (I) the amount of FOXL2 expression in the gonadal cell population is decreased; and/or (II) the amount of expression of NR1H4 and/or KITLG in the gonadal cell population is decreased; and/or (III) the amount of KRT-19 expression in the gonadal cell population is increased; and/or (IV) the amount of cytoplasmic KRT-19 expression in the gonadal cell population is increased; and/or (V) the amount of expression of MSLN, LRRN4, and/or TMEM151A in the gonadal cell population is increased as compared to a corresponding gonadal cell population generated by a method wherein the gonadal induction medium comprises a lower concentration of RAPM.

In some of any embodiments, the gonadal cell population produced by the method is one in which: (I) the amount of FOXL2 expression in the gonadal cell population is decreased; and/or (II) the amount of expression of NR1H4 and/or KITLG in the gonadal cell population is decreased; and/or (III) the amount of KRT-19 expression in the gonadal cell population is increased; and/or (IV) the amount of cytoplasmic KRT-19 expression in the gonadal cell population is increased; and/or (V) the amount of expression of MSLN, LRRN4, and/or TMEM151A in the gonadal cell population is increased, as compared to a corresponding gonadal cell population generated by a method wherein the gonadal induction medium does not comprise a RAPM.

In some of any embodiments, the gonadal cell population is one in which: (I) the amount of FOXL2 expression in the gonadal cell population is decreased; and/or (II) the amount of expression of NR1H4 and/or KITLG in the gonadal cell population is decreased; and/or (III) the amount of KRT-19 expression in the gonadal cell population is increased; and/or (IV) the amount of cytoplasmic KRT-19 expression in the gonadal cell population is increased; and/or (V) the amount of expression of MSLN, LRRN4, and/or TMEM151A in the gonadal cell population is increased, as compared to a corresponding gonadal cell population generated by a gonadal induction step comprising contacting with an RAPM for a shorter period of time.

In some of any embodiments, the pluripotent stem cells are mammalian stem cells. In some embodiments, the pluripotent stem cells are human pluripotent stem cells. In some embodiments, the pluripotent stem cells are bovine stem cells. In some embodiments, the pluripotent stem cells are murine pluripotent stem cells. In some embodiments, the pluripotent stem cells are embryonic stem cells or induced pluripotent stem cells.

In some of any embodiments, the gonadal population comprises one or more populations selected from granulosa cells, ovarian stroma cells and epithelial cells or a combination thereof. In some of any embodiments, the gonadal population comprises a mixture of granulosa cells, ovarian stroma cells and epithelial cells

Provided herein is a gonadal cell population produced by any of the provided methods. In some embodiments, the gonadal population comprises one or more populations selected from granulosa cells, ovarian stroma cells and epithelial cells or a combination thereof. In some embodiments, the gonadal population comprises a mixture of granulosa cells, ovarian stroma cells and epithelial cells

Provided herein is a first intermediate cell population produced by any of the provided methods.

Provided herein is a second intermediate cell population produced by any of the provided embodiments.

Among the provided embodiments are:
1. A method of producing a granulosa cell, the method comprising:
culturing a pluripotent stem cell in the presence of activin A, a glycogen synthase kinase-3 inhibitor, and a ROCK inhibitor to produce an incipient mesoderm-like cell (iMeLC),
culturing the iMeLC in the presence of FGF2, a glycogen synthase kinase-3 inhibitor, and a ROCK inhibitor for a first period of time,
reducing an amount of the ROCK inhibitor in the iMeLC culture and then culturing the iMeLC for a second period of time to produce an intermediate mesoderm cell, and
culturing the intermediate mesoderm cell in the presence of follistatin, BMP4, FGF2, and a ROCK inhibitor to produce the granulosa cell.
2. The method of embodiment 1, wherein the glycogen synthase kinase-3 inhibitor is CHIR99021.
3. The method of embodiment 1 or 2, wherein the ROCK inhibitor is Y-27632 or CET.
4. The method of any one of embodiments 1-3, wherein the pluripotent stem cell is cultured for about 56 to 72 hours.
5. The method of any one of embodiments 1-4, wherein the pluripotent stem cell is cultured for about 65 hours.
6. The method of any one of embodiments 1-5, wherein pluripotent stem cell is cultured in a medium comprising the activin A, glycogen synthase kinase-3 inhibitor, and ROCK inhibitor, and wherein the medium is replaced with fresh medium every about 24 hours.
7. The method of any one of embodiments 1-6, wherein the first period of time is about 24 hours.
8. The method of any one of embodiments 1-7, wherein the second period of time is about 5 or 6 days.
9. The method of any one of embodiments 1-8, wherein the iMeLC is cultured in a medium comprising the FGF2, Glycogen synthase kinase-3 inhibitor, and ROCK inhibitor for the first period of time, and wherein after the first period of time a portion of the medium is replaced with a medium comprising FGF2 and glycogen synthase kinase-3 inhibitor but not a ROCK inhibitor.
10. The method of embodiment 9, wherein the portion of the medium is about 80% of the medium.
11. The method of embodiment 9 or 10, wherein the method further comprises replacing a second portion of the medium every about 48 hours during the second time period.
12. The method of embodiment 11, wherein the second portion of the medium is about 80% of the medium.
13. The method of any one of embodiments 11-12, wherein the intermediate mesoderm cell is cultured for a period of about 5-7 days.
14. The method of any one of embodiments 11-13, wherein the intermediate mesoderm cell is cultured in a medium comprising the follistatin, BMP4, FGF2, and ROCK inhibitor, and wherein a portion of the medium is replaced with fresh medium every about 24 to 48 hours.
15. The method of any one of embodiments 11-14, wherein the iMeLC expresses Brachyury.
16. The method of any one of embodiments 11-13, wherein the intermediate mesoderm cell expresses OSR1, PAX2, and LHX1.
17. The method of any one of embodiments 11-16, wherein the granulosa cell expresses FOXL2 and CONNEXIN43.
18. The method of any one of embodiments 11-17, wherein the pluripotent stem cell is a human induced pluripotent stem cell.
19. A population comprising granulosa cells produced by the method of any one of embodiments 11-18.

In any of the provided embodiments, including embodiments of any of the provided methods or populations, (a) one or more of the culturing steps comprises adherent culture; and/or (b) one or more of the culturing steps comprises 3-dimensional organoid culture. In some embodiments, one or more of the culturing steps comprises adherent culture. In some embodiments, one or more of the culturing step comprises 3-dimensional organoid culture.

In some of any of the provided embodiments, the method is carried out in vitro. In some embodiments, the methods produce an in vitro stem cell-derived gonadal cell population. In particular embodiments, the cells are a in vitro stem cell-derived gonadal somatic cell population, such as an ovarian somatic cell population (OSC).

Also provided herein is an in vitro stem cell-derived gonadal somatic cell population comprising FOXL2-expressing cells, NR2F2-expressing cells and/or KRT-19 expressing cells. In some embodiments, the gonadal somatic cell population is an ovarian somatic cell population. In some embodiments, the population comprises at least a first cell type expressing FOXL2, a second cell type expressing NR2F2, and a third cell type expressing KRT-19. In some embodiments, at least 20% of cells within the cell population are FOXL2-positive cells. In some embodiments, at least 20% of the cells within the cell population are NR2F2-positive cells. In some embodiments, at least 20% of the cells within the gonadal cell population are KRT19-positive cells. In some embodiments, (a) at least 20% of cells within the cell population are FOXL2-positive cells, and/or (b) at least 20% of the cells within the cell population are NR2F2-positive cells; and/or (c) at least 20% of the cells within the gonadal cell population are KRT19-positive cells. In some embodiments, least 90% of the gonadal cell population are FOXL2-positive cells, NR2F2-positive cells, and/or KRT-19 positive cells; optionally wherein: the gonadal somatic cell population consists essentially of FOXL2-positive cells, NR2F2-positive cells, and/or KRT-19 positive cells. In some embodiments, the FOXL2-positive cells comprise granulosa cells; the NR2F2-positive cells comprise ovarian stromal cells and/or granulosa cells; and the KRT-19 positive cells comprise ovarian epithelial cells.

In some of any embodiments of a provided gonadal somatic cell population the cell population is differentiated from pluripotent stem cells. In some of any embodiments, the gonadal somatic cell population is derived in a process comprising: (a) culturing pluripotent stem cells in a mesoderm induction medium comprising Activin A for a first period of time to produce a first intermediate cell population; (b) culturing the first intermediate cell population in an intermediate mesoderm induction medium comprising an retinoic acid pathway modulator (RAPM) for a second period of time, thereby producing a second intermediate cell population; and (c) culturing the second intermediate cell population in a gonadal induction medium comprising BMP and FGF, and optionally follistatin, for a third period of time to produce the gonadal cell population.

In some of any embodiments, the gonadal somatic cell population is or has been cryopreserved. In some embodiments, there is provided a composition comprising a gonadal somatic cell population. In some embodiments, the composition further comprises a cryoprotectant.

### BRIEF DESCRIPTION OF THE DRAWINGS

Representative embodiments of the invention are disclosed by reference to the following figures. It should be understood that the embodiments depicted are not limited to the precise details shown.

FIG. 1A and FIG. 1B show the characterization of mesoderm induction, illustrating the morphology of iMeLCs (FIG. 1A): Staining iMeLCs and un-induced cells with fluorescently labeled antibody against Brachyury (FIG. 1B).

FIGS. 2A-2D show the characterization of intermediate mesoderm induction, illustrating Morphology of IM cells at day5 of culture (FIG. 2A); Expression pattern for OSR1 across iPSCs, iMeLCs and IMs during differentiation (FIG. 2B); Expression pattern for PAX2 across iPSCs, iMeLCs and IMs during differentiation (FIG. 2C); and Expression pattern for LHX1 across iPSCs, iMeLCs and IMs during differentiation (FIG. 2D).

FIGS. 3A-3C show the characterization of gonadal cell differentiation, illustrating the morphology of gonadal cells at day 7 of culture (FIG. 3A), Staining for FOXL2-transcription factor that is highly expressed in granulosa cells (FIG. 3B); representative immunofluorescence images for IM, kidney and granulosa cells that are stained for FOXL2 (FIG. 3C second row) and connexin43 (FIG. 3C third row).

FIG. 4 shows the CD24 expression pattern during in vitro gonadal cell differentiation from iPSCs.

FIGS. 5A-C show the effect of retinoic acid (RA) in IM induction. RA/TTNPB treatment led to increased cell survival and uniform cellular morphology (FIG. 5A) Expression of IM marker-LHX1. PAX2 shows higher expression with increasing RA and CHIR concentration (FIG. 5B). Additional markers of IM lineage-WT1 and RUNX1 show similar increasing expression with increasing RA or TTNPB concentration (FIG. 5C).

FIGS. 6A-6E show that Retinoic acid (RA) treated IM cells exhibited better survival and induction of gonadal somatic cell differentiation. FIG. 6A illustrates a bulk RNAseq experiment without RA treatment at any stage, in vitro gonadal somatic cells exhibited increased expression of granulosa cell markers-FOXL2, RUNX1, NR2F2, WNT6 and KRT19. FIG. 6B shows that in long term cultures of granulosa cells, RA treated cells exhibited better survival and higher expression of FOXL2 and RUNX1. FIG. 6C are immunofluorescence stainings of granulosa cells for FOXL2 and NR2F2. FIG. 6D shows that continuous treatment with RA during differentiation of the second intermediate cell population led to increased expression of KRT19. FIG. 6E shows the Immunofluorescence staining for KRT19 on day 28 of the gonadal somatic cell culture.

FIG. 7 shows the qRT-PCR of granulosa cell marker Fox12, ovarian stromal cells Nr2f2 and ovarian epithelial cells KRT19. Expression at day 13 of the murine differentiation protocol was compared to the basic granulosa basal medium with addition of or Retinoic Acid (RA). Positive and negative controls are included as e13 mouse ovary and mPSCs respectively.

FIG. 8 shows the Immuno-fluorescent staining on day 13 of the murine differentiation protocol after 7 days of granulosa induction showing the number of cells and level of expression of Fox12, NR2F2 and KRT19 with and without 1 μM of Retinoic Acid treatment added in addition to the granulosa basal medium growth factors.

FIG. 9A shows the relative expression levels of markers of bi-potent ovarian somatic cell progenitors after ovarian somatic cell (OSC) induction with different FGF family members. FIG. 9B shows the relative expression levels of markers of mature ovarian granulosa cells after OSC induction with different FGF family members.

FIG. 10A shows the relative expression levels of markers of bi-potent ovarian somatic cell progenitors after OSC induction with different BMP isoforms. FIG. 10B shows the relative expression levels of markers of mature ovarian granulosa cells after OSC induction with different BMP isoforms.

FIG. 11A shows the relative expression of steroidogenic enzyme CYP19A1 at different stages of OSC differentiation (iPSC stage; IM D5-intermediate mesoderm Day 5; OSC D7 basic-Day 7 of OSC induction in granulosa basic media: OSC D7 Double-Day 7 of OSC induction in granulosa double medi). FIG. 11B shows the secreted estradiol when the OSCs were treated with the indicated amount of dhT for 24 hours (left four bars) or 48 hours (right two bars).

FIGS. 12A, 12C and 12E shows the relative expression levels of GATA4, marker of bi-potent ovarian somatic cell progenitors after OSC induction with the indicated amount of follistatin (Foll). FGF2 (FGF) and BMP4 (BMP). FIGS. 12B, 12D, 12F show the relative expression levels of FOXL2, marker of mature ovarian granulosa cells after OSC induction with the indicated amount of follistatin (Foll), FGF2 (FGF) and BMP4 (BMP).

FIG. 13A displays a panel of immunofluorescence images indicating the expression of granulosa marker FOXL2 and ovarian epithelial marker KRT19 when OSC is induced in the absence of retinoic acid (RA). FIG. 13B displays a panel of immunofluorescence images indicating the expression of granulosa marker FOXL2 and ovarian epithelial marker KRT19 when OSC is induced in the presence of 500 nM retinoic acid (RA). FIGS. 13C and 13D show the relative expression levels of FOXL2, marker for granulosa cells and KRT19, marker for ovarian epithelial cells, respectively, when OSC is induced in RA of the indicated concentrations. FIGS. 13E and 13F show the relative expression levels of FOXL2, marker for granulosa cells and KRT19, marker for ovarian epithelial cells, respectively, when OSC is induced in the presence of RA at the indicated concentrations, for the indicated durations.

FIGS. 14A and 14B show the heatmap for the top 200 highly variable genes and principal component analysis of the normalized and processed data, respectively, indicating the changes in the gene expression profile during transition between from the first intermediate stage to the second intermediate stages stage, and from the second intermediate stage to the OSCs. FIG. 14C shows the distinct clusters of genes with increased expression that were observed amongst groups of more than 10.000 cells analyzed in this experiment. FIGS. 14D, 14E, 14F, 14G show the expression bipotential gonad somatic cell markers, the ovarian stromal markers, the granulosa markers, and ovarian epithelial markers, respectively of cells at Day 7 and day 14 of gonadal somatic induction (OSC induction) with either basic granulosa media (Basic), double granulosa media (Double) and with or without retinoic acid treatment (RA).

FIG. 15 shows the immunofluorescence images indicating the presence of ovarian somatic cells (GATA4 and NR5A1 expressing cells), as well as ovarian epithelial/mesothelial cells (KRT19 expressing cells).

### DETAILED DESCRIPTION OF THE INVENTION

In some aspects, the present disclosure relates to methods of generating gonadal cell populations such as ovarian somatic cells from pluripotent stem cells. Also included are gonadal cell populations, as well as intermediate cell populations generated therein.

All publications, including patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### General Techniques

The techniques and procedures described or referenced herein are generally well understood and commonly employed using conventional methodology by those skilled in the art, such as, for example, the widely utilized methodologies described in Molecular Cloning: A Laboratory Manual (Sambrook et al . . . 4^{th} ed., Cold Spring Harbor Laboratory Press, Cold Spring Harbor, N.Y., 2012); Current Protocols in Molecular Biology (F. M. Ausubel, et al. eds., 2003); the series Methods in Enzymology (Academic Press, Inc.); PCR 2: A Practical Approach (M. J. MacPherson, B. D. Hames and G. R. Taylor eds., 1995); Antibodies, A Laboratory Manual (Harlow and Lane, eds., 1988); Culture of Animal Cells: A Manual of Basic Technique and Specialized Applications (R. I. Freshney, 6^{th} ed., J. Wiley and Sons. 2010); Oligonucleotide Synthesis (M. J. Gait, ed., 1984); Methods in Molecular Biology, Humana Press; Cell Biology: A Laboratory Notebook (J. E. Cellis, ed., Academic Press, 1998); Introduction to Cell and Tissue Culture (J. P. Mather and P. E. Roberts, Plenum Press, 1998); Cell and Tissue Culture: Laboratory Procedures (A. Doyle, J. B. Griffiths, and D. G. Newell, eds., J. Wiley and Sons. 1993-8); Handbook of Experimental Immunology (D. M. Weir and C. C. Blackwell, eds., 1996); Gene Transfer Vectors for Mammalian Cells (J. M. Miller and M. P. Calos, eds., 1987); PCR: The Polymerase Chain Reaction, (Mullis et al., eds., 1994); Current Protocols in Immunology (J. E. Coligan et al., eds., 1991); Short Protocols in Molecular Biology (Ausubel et al., eds., J. Wiley and Sons, 2002); Immunobiology (C. A. Janeway et al., 2004); Antibodies (P. Finch, 1997); Antibodies: A Practical Approach (D. Catty, ed., IRL Press, 1988-1989); Monoclonal Antibodies: A Practical Approach (P. Shepherd and C. Dean, eds., Oxford University Press, 2000); Using Antibodies: A Laboratory Manual (E. Harlow and D. Lane, Cold Spring Harbor Laboratory Press, 1999): The Antibodies (M. Zanetti and J. D. Capra, eds., Harwood Academic Publishers, 1995); and Cancer: Principles and Practice of Oncology (V. T. DeVita et al., eds., J.B. Lippincott Company, 2011)

### Definitions

For purposes of interpreting this specification, the following definitions will apply and whenever appropriate, terms used in the singular will also include the plural and vice versa. In the event that any definition set forth below conflicts with any document incorporated herein by reference, the definition set forth shall control.

As used herein, the singular form “a”, “an”, and “the” includes plural references unless indicated otherwise.

It is understood that aspects and embodiments of the invention described herein include “comprising,” “consisting,” and “consisting essentially of” aspects and embodiments.

The term “about” as used herein refers to the usual error range for the respective value readily known to the skilled person in this technical field. Reference to “about” a value or parameter herein includes (and describes) embodiments that are directed to that value or parameter per se.

The term “homogenous” as used herein refers to something which is consistent or uniform in structure or composition throughout. In some examples, the term refers to cells having consistent maturation status, marker expression or phenotype within a given population.

As used herein, the term “inhibit” may refer to the act of blocking, reducing, eliminating, or otherwise antagonizing the presence, or an activity of, a particular target. For example, inhibiting the phosphorylation of Tau protein may refer to any act leading to decreasing, reducing, antagonizing eliminating, blocking or otherwise diminishing the phosphorylation of Tau protein. Inhibition may refer to partial inhibition or complete inhibition. In other examples, inhibition of the expression of a nucleic acid may include, but not limited to reduction in the transcription of a nucleic acid, reduction of mRNA abundance (e.g., silencing mRNA transcription), degradation of mRNA, inhibition of mRNA translation, and so forth.

As used herein, the term “suppress” may refer to the act of decreasing, reducing, prohibiting, limiting, lessening, or otherwise diminishing the presence, or an activity of, a particular target. Suppression may refer to partial suppression or complete suppression. For example, suppressing phosphorylation of Tau protein may refer to any act leading to decreasing, reducing, prohibiting, limiting, lessening, or otherwise diminishing the phosphorylation of Tau protein. In other examples, suppression of the expression of a nucleic acid may include, but not limited to reduction in the transcription of a nucleic acid, reduction of mRNA abundance (e.g., silencing mRNA transcription), degradation of mRNA, inhibition of mRNA translation, and so forth.

As used herein, the term “enhance” may refer to the act of improving, boosting, heightening, or otherwise increasing the presence, or an activity of, a particular target. For example, enhancing steroidogenesis may refer to any act leading to improving, boosting, heightening, or otherwise increasing steroidogenesis.

As used herein, the term “modulate” may refer to the act of changing, altering, varying, or otherwise modifying the presence, or an activity of, a particular target. For example, modulating a signaling pathway may include but not limited to any acts leading to changing, altering, varying, or otherwise modifying the activity of the signaling pathway. In some examples, “modulate” refers to enhancing the presence or activity of a particular target. In some examples, “modulate” refers to suppressing the presence or activity of a particular target. For example, modulating the amount of retinoic acid signaling may include but is not limited to suppressing or enhancing the amount of the retinoic acid signaling.

As used herein, the term “induce” may refer to the act of initiating, prompting, stimulating, establishing, or otherwise producing a result. For example, inducing an expression of mutant gene may refer to any act leading to initiating, prompting, stimulating, establishing, or otherwise producing the desired expression of the mutant gene. In other examples, inducing the expression of a nucleic acid may include, but not limited to initiation of the transcription of a nucleic acid, initiation of mRNA translation, and so forth. In some examples, inducing a germ layer may refer to any act leading to or designed for leading to initiating, prompting, stimulating, establishing, or otherwise producing the desired derivation of the germ layer.

As used herein “stem cell”, unless defined further, refers to any non-somatic cell. Any cell that is not a terminally differentiated or terminally committed cell may be referred to as a stem cell. This includes embryonic stem cells, induced pluripotent stem cells, hematopoietic stem cells, progenitor cells, and partially differentiated progenitor cells. Stem cells may be totipotent, pluripotent, or multipotent stem cells. Any cell which has the potential to differentiate into two different types of cells is considered a stem cell for the purpose of this application.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

For any of the structural and functional characteristics described herein, methods of determining these characteristics are known in the art.

### Derivation, Differentiation and Maturation of Gonadal Progeny Cells

Human iPSCs have become powerful tools in modeling human diseases and hold tremendous potential for translational research in target discovery and drug development.

While recent advances have indicated that oocytes could be generated from pluripotent stem cells in vitro, these derived cells still require a proper somatic environment to develop fully as reproductive cells. It is only when combined with the ovarian somatic cells of the correct makeup that early primordial germ cells or in vitro-derived primordial germ cell-like cells mature to gametes. Provided herein are methods of producing gonadal cell populations, such as in particular ovarian somatic cells, from pluripotent stem cells. In some embodiments, the provided cell populations can be used in methods to support oocyte development in vitro, such as in connection with in vitro gametogenesis.

Differentiation of stem cells progresses through various stages that can be identified by changes in gene expression. Described is a method to generate gonadal cells (such as ovarian somatic cells) by progressively converting stem cells through a series of steady stable states. In some embodiments, conditions were optimized for maximum purity and efficiency in order to obtain pure homogeneous ovarian somatic cell cultures. In other embodiments, conditions were optimized to obtain certain heterogeneous ovarian somatic cell cultures comprising granulosa cells, ovarian stromal cells and ovarian epithelial cells. In some embodiments, provided herein are two key intermediate steps for granulosa cell differentiation. In some embodiments, provided herein are two key intermediate steps for ovarian somatic cell differentiation. In some embodiments, the two key intermediate steps include-differentiating pluripotent stem cells first into a mesoderm intermediate, then into intermediate mesoderm population, before driving the cells into gonadal cell populations (such as ovarian somatic cells, OSCs).

In some embodiments, the protocol described herein can be employed for different stem cell lines, from different mammals (such as but not limited to human, mouse bovine). For robust differentiation, the expression of certain markers may be optimized at each step by changing the concentration and the duration of inducers (e.g., cytokines or small molecules). More importantly, since a precise balance and organization of different ovarian somatic cell types (such as but not limited to granulosa cells, epithelial cells and stromal cells) may be necessary to form a functional “mini-ovary” to facilitate gamete production and maturation, the final composition of ovarian somatic cells are optimized by changing the concentration and the duration of inducers. Findings herein support that use of different concentrations of particular compounds (such as but not limited to retinoic acid and glycogen synthase kinase 3), as well as different timings of incubation during induction of intermediate mesoderm and/or induction of gonadal cell populations were shown to produce gonadal cell populations that include various assemblies of ovarian somatic cells, including granulosa cells, epithelial cells and stromal cells. Such optimization, as demonstrated in the specification herein, is built into the protocol for a robust, universal method that would work with cell lines of different genetic backgrounds. Such methods and cell populations could also tailor and facilitate the development and maturation of pluripotent stem cell-derived oocytes generated by different protocols.

### Deriving Gonadal Cell Populations

### Methods of Generating Gonadal Cell Populations and Intermediate Cell Populations

In some aspects, provided are methods of generating gonadal somatic cell populations from pluripotent stem cells. In some embodiments, provided herein is a method of producing a gonadal somatic cell population, the method comprising: (a) culturing pluripotent stem cells in a mesoderm induction medium comprising activin A for a first period of time to produce a first intermediate cell population: (b) culturing the first intermediate cell population in an intermediate mesoderm induction medium comprising a retinoic acid pathway modulator (RAPM) for a second period of time, thereby producing a second intermediate cell population; and (c) culturing the second intermediate cell population in a gonadal induction medium comprising BMP and FGF, for a third period of time to produce the gonadal cell population.

In some embodiments, the gonadal induction medium further comprises follistatin.

In some aspects, provided are methods of generating gonadal cell populations from pluripotent stem cells. In some embodiments, provided herein is a method of producing a gonadal cell population, the method comprising: (a) culturing pluripotent stem cells in a mesoderm induction medium comprising activin A for a first period of time to produce a first intermediate cell population; (b) culturing the first intermediate cell population in an intermediate mesoderm induction medium comprising a retinoic acid pathway modulator (RAPM) for a second period of time, thereby producing a second intermediate cell population; and (c) culturing the second intermediate cell population in a gonadal induction medium comprising follistatin, BMP4 and FGF, for a third period of time to produce the gonadal cell population.

In some embodiments, provided is a method of producing a gonadal cell population, the method comprising: (a) culturing mesoderm or mesoderm-like cells in an intermediate mesoderm induction medium comprising a RAPM for a first period of time thereby producing a second intermediate cell population; and (b) culturing the second intermediate cell population in a gonadal induction medium comprising BMP and FGF for a second period of time to produce the gonadal cell population. In some embodiments, the gonadal induction medium further comprises follistatin. In some embodiments, provided is a method of producing a gonadal cell population, the method comprising: (a) culturing mesoderm or mesoderm-like cells in an intermediate mesoderm induction medium comprising a RAPM for a first period of time thereby producing a second intermediate cell population; and (b) culturing the second intermediate cell population in a gonadal induction medium comprising follistatin, BMP4, and FGF for a second period of time to produce the gonadal cell population.

In some embodiments, provided is a method of producing a gonadal cell population, the method comprising: culturing intermediate mesoderm or intermediate mesoderm-like cells in a gonadal induction medium comprising BMP and FGF for a period of time to produce the gonadal cell population. In some embodiments, the gonadal induction medium further comprises follistatin. In some embodiments, provided is a method of producing a gonadal cell population, the method comprising: culturing intermediate mesoderm or intermediate mesoderm-like cells in a gonadal induction medium comprising follistatin, BMP4 and FGF for a period of time to produce the gonadal cell population.

In some embodiments, provided is a method of producing a first intermediate cell population, the method comprising: culturing pluripotent stem cells in a mesoderm induction medium comprising activin A and a glycogen synthase kinase-3 inhibitor for a period of time to produce a first intermediate cell population

In some embodiments, provided is a method of producing a second intermediate cell population, the method comprising: culturing mesoderm or mesoderm-like cells in a medium comprising RAPM for a period of time, thereby producing the second intermediate cell population.

In some embodiments, provided is a method of producing a second intermediate cell population, the method comprising: (a) culturing pluripotent stem cells in a medium comprising activin A and a glycogen synthase kinase-3 inhibitor for a first period of time to produce a first intermediate cell population; (b) culturing the first intermediate cell population in a medium comprising a RAPM, FGF, and a glycogen synthase kinase-3 inhibitor for a second period of time, thereby producing the second intermediate cell population.

In some embodiments according to any one of the methods described herein,

### Gonadal Cell Populations and Intermediate Cell Populations

In some aspects, provided is an in vitro stem cell-derived gonadal somatic cell population comprising FOXL2-expressing cells, NR2F2-expressing cells and/or KRT-19 expressing cells, optionally wherein the gonadal somatic cell population is an ovarian somatic cell population. In some embodiments, the gonadal somatic cell population is generated by a process comprising: (a) culturing pluripotent stem cells in a mesoderm induction medium comprising activin A for a first period of time to produce a first intermediate cell population; (b) culturing the first intermediate cell population in an intermediate mesoderm induction medium comprising a retinoic acid pathway modulator (RAPM) for a second period of time, thereby producing a second intermediate cell population; and (c) culturing the second intermediate cell population in a gonadal induction medium comprising BMP and FGF, for a third period of time, thereby generating the gonadal cell population. In some embodiments, the gonadal induction medium further comprises follistatin.

Also provided are gonadal cell populations, as well as intermediate cell populations generated by the methods described herein. In some embodiments, provided herein is a gonadal cell population, generated by a method comprising: (a) culturing pluripotent stem cells in a mesoderm induction medium comprising activin A for a first period of time to produce a first intermediate cell population: (b) culturing the first intermediate cell population in an intermediate mesoderm induction medium comprising a retinoic acid pathway modulator (RAPM) for a second period of time, thereby producing a second intermediate cell population; and (c) culturing the second intermediate cell population in a gonadal induction medium comprising follistatin, BMP4 and FGF, for a third period of time, thereby generating the gonadal cell population. In some embodiments, provided herein is a gonadal cell population, generated by a method comprising: (a) culturing pluripotent stem cells in a mesoderm induction medium comprising activin A for a first period of time to produce a first intermediate cell population: (b) culturing the first intermediate cell population in an intermediate mesoderm induction medium comprising a retinoic acid pathway modulator (RAPM) for a second period of time, thereby producing a second intermediate cell population; and (c) culturing the second intermediate cell population in a gonadal induction medium comprising BMP and FGF, for a third period of time, thereby generating the gonadal cell population. In some embodiments, the gonadal induction medium further comprises follistatin.

In some embodiments, provided herein is a gonadal cell population, generated by a method comprising: (a) culturing mesoderm or mesoderm-like cells in an intermediate mesoderm induction medium comprising a RAPM for a first period of time thereby producing a second intermediate cell population; and (b) culturing the second intermediate cell population in a gonadal induction medium comprising follistatin, BMP4, and FGF for a second period of time, thereby generating the gonadal cell population. In some embodiments, provided herein is a gonadal cell population, generated by a method comprising: (a) culturing mesoderm or mesoderm-like cells in an intermediate mesoderm induction medium comprising a RAPM for a first period of time thereby producing a second intermediate cell population; and (b) culturing the second intermediate cell population in a gonadal induction medium comprising BMP, and FGF for a second period of time, thereby generating the gonadal cell population. In some embodiments, the gonadal induction medium further comprises follistatin.

In some embodiments, provided is a gonadal cell population, generated by a method comprising: culturing intermediate mesoderm or intermediate mesoderm-like cells in a gonadal induction medium comprising follistatin, BMP4 and FGF for a period of time to produce the gonadal cell population. In some embodiments, provided is a gonadal cell population, generated by a method comprising: culturing intermediate mesoderm or intermediate mesoderm-like cells in a gonadal induction medium comprising BMP and FGF for a period of time to produce the gonadal cell population. In some embodiments, the gonadal induction medium further comprises follistatin.

In some embodiments, provided a first intermediate cell population, generated by a method comprising: culturing pluripotent stem cells in a mesoderm induction medium comprising activin A and a glycogen synthase kinase-3 inhibitor for a period of time, thereby generating a first intermediate cell population.

In some embodiments, provided is a second intermediate cell population, the method comprising: culturing mesoderm or mesoderm-like cells in a medium comprising RAPM for a period of time, thereby producing the second intermediate cell population.

In some embodiments, provided is a second intermediate cell population, generated by a method comprising: (a) culturing pluripotent stem cells in a medium comprising activin A and a glycogen synthase kinase-3 inhibitor for a first period of time to produce a first intermediate cell population; (b) culturing the first intermediate cell population in a medium comprising a RAPM, FGF, and a glycogen synthase kinase-3 inhibitor for a second period of time, thereby producing the second intermediate cell population.

### Induction Medium, Differentiation Protocols and Precursor Cells

In some embodiments according to any one of the methods or cell populations described herein, the pluripotent stem cells are human pluripotent stem cells or murine pluripotent stem cells. In some embodiments, the pluripotent stem cells are embryonic stem cells or induced pluripotent stem cells. In some embodiments, the pluripotent stem cells are mammalian pluripotent stem cells. In some embodiments, the pluripotent stem cells are murine or human pluripotent stem cells. In some embodiments, the pluripotent stem cells are murine pluripotent stem cells. In some embodiments, the pluripotent stem cells are human pluripotent stem cells. In some embodiments, the pluripotent stem cells are bovine pluripotent stem cells.

In some embodiments, the pluripotent stem cells are seeded at a density of about 10,000 to about 40,000 cells per cm². In some embodiments,

In some embodiments according to any one of the methods or cell populations described herein, the concentration of Activin A in the mesoderm induction medium is about 30 ng/mL to about 70 ng/mL. In some embodiments, the concentration of Activin A in the mesoderm induction medium is about 50 ng/mL. In some embodiments, the concentration of Activin A in the mesoderm induction medium is about any one of: 5, 10, 15, 20, 25, 30, 35, 40, 45, 50, 55, 60, 65, 70, 75, 80, 85, 90, 95, 100, 125, 150, 200, 300, 400 or 500 ng/mL, or one of any concentrations there between. In some embodiments the concentration of Activin A in the mesoderm induction medium is about any one of about 30, 32, 34, 36, 38, 40, 42, 44, 46, 48, 50, 52, 54, 56, 58, 60, 62, 64, 66, 68 or 70 ng/mL, or one of any concentrations there between.

In some embodiments according to any one of the methods or cell populations described herein, the mesoderm induction medium further comprises FGF. In some embodiments, the FGF is one or more of: FGF2, FGF4 or FGF9, In some embodiments, the FGF is FGF2. In some embodiments, the mesoderm induction medium comprises FGF2, wherein the concentration of FGF2 is about any one of: 0.5, 1, 2, 3, 4, 5, 6, 7, 8, 9, 10, 11, 12, 13, 14, 15, 16, 17, 18, 19, 20, 21, 22, 23, 24, 25, 30, 35, 40, 50, 60, 70, 80, 90, 100 ng/ml, or one of any concentrations there between. In some embodiments, the concentration of FGF2 in the mesoderm induction medium is about 5 ng/mL to about 20 ng/mL. In some embodiments, the concentration of FGF2 in the mesoderm induction medium is about 12 ng/mL.

In some embodiments according to any one of the methods or cell populations described herein, the mesoderm induction medium further comprises BMP4. In some embodiments, the concentration of BMP4 in the mesoderm induction medium is about any one of: 5, 10, 15, 20, 25, 30, 35, 40, 45, 50, 55, 60, 65, 70, 75, 80, 85, 90, 95, 100, 125, 150, 200, 300, 400 or 500 ng/ml, or one of any concentrations there between. In some embodiments, the concentration of BMP4 in the mesoderm induction medium is about any one of about 10, 12, 14, 16, 18, 20, 22, 24, 26, 28, 30, 32, 34, 36, 38, 40, 42, 44, 46, 48 or 50 ng/mL, or one of any concentrations there between. In some embodiments, the concentration of BMP4 in the mesoderm induction medium is about 10 ng/ml to about 50 ng/mL. In some embodiments, the concentration of BMP4 in the mesoderm induction medium is about 30 ng/ml.

In some embodiments according to any one of the methods or cell populations described herein, the mesoderm induction medium further comprises a glycogen synthase kinase-3 inhibitor. In some embodiments, the glycogen synthase kinase-3 inhibitor is CHIR99021. In some embodiments, the concentration of CHIR99021 in the mesoderm induction medium is about any one of: 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 5.0, 6.0, 7.0, 8.0, 9.0, 10.0, 20.0, 30.0, 40.0, 50.0 μM, or one of any concentrations there between. In some embodiments, the concentration of CHIR99021 is about 1 μM to about 5 μM. In some embodiments, the concentration of CHIR99021 is about 3 μM.

In some embodiments according to any one of the methods or cell populations described herein, the mesoderm induction medium further comprises an apoptosis inhibitor. In some embodiments, the apoptosis inhibitor in the mesoderm induction medium is Y-27632. In some embodiments, the concentration of Y-27632 is about any one of: 0.5, 1, 2, 3, 4, 5, 6, 7, 8, 9, 10, 11, 12, 13, 14, 15, 16, 17, 18, 19, 20, 21, 22, 23, 24, 25, 30, 35, 40, 50, 60, 70, 80, 90, 100 ng/mL, or one of any concentrations there between. In some embodiments, the concentration of Y-27632 is about 5 μM to about 20 μM. In some embodiments, the concentration of Y-27632 is about 10 μM. In some embodiments, the apoptosis inhibitor in the mesoderm induction medium comprises Chroman1. Emricasan, and Trans-ISRIB. In some embodiments, the concentration of Chroman 1 is about 30 nM to about 70 nM, the concentration of Emricasan is about 2 μM to about 10 μM, and/or the concentration of Trans-ISRIB is about 0.2 μM to about 2 μM. In some embodiments, the concentration of Chroman1 is about 50 nM, the concentration of Emricasan is about 5 μM, and/or the concentration of Trans-ISRIB is about 0.7 μM.

In some embodiments according to any one of the methods or cell populations described herein, the period of time for culturing in mesoderm induction medium is about any one of: 12, 16, 20, 24, 28, 32, 36, 40, 44, 48, 52, 56, 60, 64, 68, 72, 76, 80, 84, 88, 92, 96, 120, 144, 168, 192 hours, or one of any lengths there between. In some embodiments, the period of time for culturing in mesoderm induction medium is at least about any one of: 12, 16, 20, 24, 28, 32, 36, 40, 44, 48, 52, 56, 60, 64, 68, 72, 76, 80, 84, 88, 92, 96, 120, 144, 168, 192 hours. In some embodiments, the period of time for culturing in mesoderm induction medium is about 24 hours to about 96 hours. In some embodiments, the period of time for culturing in mesoderm induction medium is about 24 hours to about 72 hours. In some embodiments, the period of time for culturing in mesoderm induction medium is about 56 hours to about 72 hours.

In some embodiments, the first intermediate cell population is replated onto a new fibronectin-coated culture plate prior to culturing in intermediate mesoderm induction medium: optionally wherein the first intermediate cell population is enzymatically detached, centrifuged and resuspended before replating. In some embodiments, the first intermediate cell population is plated at a density of about 5000 to about 25000 cells/cm². In some embodiments, the first intermediate cell population is plated at a density of about 25000 to about 75000 cells/cm². In some embodiments, the first intermediate cell population is plated at a density of about 75000 to about 150000 cells/cm². In some embodiments, the mesoderm or mesoderm-like cells are plated at a density of about 5000 to about 25000 cells/cm². In some embodiments, the mesoderm or mesoderm-like cells are plated at a density of about 25000 to about 75000 cells/cm². In some embodiments, the mesoderm or mesoderm-like cells are plated at a density of about 75000 to about 150000 cells/cm².

In some embodiments according to any one of the methods or cell populations described herein, the RAPM in the intermediate mesoderm induction medium is an RAR agonist. In some embodiments, the RAPM comprises retinoic acid (RA) and/or TTNPB. In some embodiments, the RAPM is RA. In some embodiments, the RAPM is TTNPB. In some embodiments, the concentration of RA in the intermediate mesoderm induction medium is about any one of: 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 5.0, 6.0, 7.0, 8.0, 9.0, 10.0, 20.0, 30.0, 40.0, 50.0 μM, or one of any concentrations there between. In some embodiments, the concentration of TTNPB is about any one of: 0.005, 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 5.0, 6.0, 7.0, 8.0, 9.0, 10.0, 20.0 μM, or one of any concentrations there between. In some embodiments, the concentration of RA in the intermediate mesoderm induction medium is about 0.5 μM to about 2 μM, and/or the concentration of TTNPB in the intermediate mesoderm induction medium is about 0.2 μM to about 1 μM. In some embodiments, the concentration of RA in the intermediate mesoderm induction medium is about 1 μM; and/or the concentration of TTNPB in the intermediate mesoderm induction medium is about 0.5 μM.

In some embodiments according to any one of the methods or cell populations described herein, the intermediate mesoderm induction medium further comprises FGF. In some embodiments, the FGF is one or more of: FGF2, FGF4 or FGF9, In some embodiments, the FGF is FGF2. In some embodiments, the intermediate mesoderm induction medium comprises FGF2, wherein the concentration of FGF2 is about any one of: 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 50, 60, 70, 80, 90, 100 ng/ml, or any one concentrations there between. In some embodiments, the concentration of FGF2 in the intermediate mesoderm induction medium is about 10 ng/mL to about 30 ng/mL. In some embodiments, the concentration of FGF2 in the intermediate mesoderm induction medium is about 20 ng/mL.

In some embodiments according to any one of the methods or cell populations described herein, the intermediate mesoderm induction medium further comprises a glycogen synthase kinase-3 inhibitor. In some embodiments, the glycogen synthase kinase-3 inhibitor is CHIR99021. In some embodiments, the concentration of CHIR99021 in the intermediate mesoderm induction medium is about any one of: 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 5.0, 6.0, 7.0, 8.0, 9.0, 10.0, 20.0, 30.0, 40.0, 50.0 μM, or one of any concentrations there between. In some embodiments, the concentration of CHIR99021 is about 1 μM to about 5 μM. In some embodiments, the concentration of CHIR99021 is about 3 μM.

In some embodiments according to any of the methods or cell populations described herein, different compositions of the final ovarian somatic cell population can be achieved at differing concentrations and exposure duration of inducers (such as growth factors or small molecules).

In some embodiments, different concentrations of and exposure durations to RAPM and/or glycogen synthase kinase-3 inhibitor can be utilized in the derivation of the second intermediate cell population. In some embodiments, the developmental potential of the second intermediate cell population generated thereby is rendered different when different concentrations of and exposure duration to RAPM and/or glycogen synthase kinase-3 inhibitor are employed during derivation. In some embodiments, the RAPM comprises retinoic acid (RA) and/or TTNPB. In some embodiments, the RAPM is RA. In some embodiments, the RAPM is TTNPB.

In some embodiments according to any one of the methods or cell populations described herein, the intermediate mesoderm induction medium further comprises an apoptosis inhibitor. In some embodiments, the apoptosis inhibitor in the intermediate mesoderm induction medium is Y-27632. In some embodiments, the concentration of Y-27632 is about any one of: 0.5, 1, 2, 3, 4, 5, 6, 7, 8, 9, 10, 11, 12, 13, 14, 15, 16, 17, 18, 19, 20, 21, 22, 23, 24, 25, 30, 35, 40, 50, 60, 70, 80, 90, 100 ng/mL, or one of any concentrations there between. In some embodiments, the concentration of Y-27632 is about 5 μM to about 20 μM. In some embodiments, the concentration of Y-27632 is about 10 μM. In some embodiments, the apoptosis inhibitor in the intermediate mesoderm induction medium comprises Chroman1, Emricasan, and Trans-ISRIB. In some embodiments, the concentration of Chroman1 is about 30 nM to about 70 nM, the concentration of Emricasan is about 2 μM to about 10 μM, and/or the concentration of Trans-ISRIB is about 0.2 μM to about 2 μM. In some embodiments, the concentration of Chroman1 is about 50 nM, the concentration of Emricasan is about 5 μM, and/or the concentration of Trans-ISRIB is about 0.7 μM. In some embodiments, the method comprises culturing the first intermediate cell population: (i) first in the intermediate mesoderm induction medium comprising about 10 μM of Y-27632, and (ii) subsequently in the intermediate mesoderm induction medium with no more than about 2 μM of Y-27632. In some embodiments, the method comprises culturing the first intermediate cell population: (i) first in the intermediate mesoderm induction medium comprising about 10 μM of Y-27632 for about 24 hours, (ii) subsequently in the intermediate mesoderm induction medium with no more than about 2 μM of Y-27632 for about 5-6 days.

In some embodiments according to any one of the methods or cell populations described herein, the period of time for culturing in intermediate mesoderm induction medium is about any one of: 2, 3, 4, 5, 6, 7, 8, 9, 10, 11, 12, 13, 14, 15, 16, 17, 18, 19, 20, 21, 22, 23, 24, 25, 28, 30, 35, 42, 49 days or one of any lengths there between. In some embodiments, the period of time for culturing in intermediate mesoderm induction medium is at least about any one of: 2, 3, 4, 5, 6, 7, 8, 9, 10, 11, 12, 13, 14, 15, 16, 17, 18, 19, 20, 21, 22, 23, 24, 25, 28, 30, 35, 42, 49 days. In some embodiments, the period of time for culturing in intermediate mesoderm induction medium is about 4 days to about 14 days. In some embodiments, the period of time for culturing in intermediate mesoderm induction medium is about 5 days to about 9 days.

In some embodiments, the second intermediate cell population is replated onto a new fibronectin-coated culture plate prior to culturing in gonadal induction medium; optionally wherein the second intermediate cell population is enzymatically detached, centrifuged and resuspended before replating. In some embodiments, the second intermediate cell population is plated at a density of about 5000 to about 25000 cells/cm². In some embodiments, the second intermediate cell population is plated at a density of about 25000 to about 75000 cells/cm². In some embodiments, the second intermediate cell population is plated at a density of about 75000 to about 150000 cells/cm². In some embodiments, the mesoderm or mesoderm-like cells are plated at a density of about 5000 to about 25000 cells/cm². In some embodiments, the intermediate mesoderm or intermediate mesoderm-like cells are plated at a density of about 5000 to about 25000 cells/cm². In some embodiments, the intermediate mesoderm or intermediate mesoderm-like cells are plated at a density of about 25000 to about 75000 cells/cm². In some embodiments, the intermediate mesoderm or intermediate mesoderm-like cells are plated at a density of about 75000 to about 150000 cells/cm².

In some embodiments according to any one of the methods or cell populations described herein, the concentration of follistatin in the gonadal induction medium is about any one of: 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 40, 50, 60, 70, 80, 90, 100, 200, 500 ng/ml, or one of any concentrations there between. In some embodiments, the concentration of follistatin in the gonadal induction medium is about 10 ng/ml to about 50 ng/ml. In some embodiments, the concentration of follistatin in the gonadal induction medium about 25 ng/mL.

In some embodiments according to any one of the methods or cell populations described herein, the concentration of BMP4 in the gondal induction medium is about any one of: 5, 10, 15, 20, 25, 30, 35, 40, 45, 50, 55, 60, 65, 70, 75, 80, 85, 90, 95, 100, 125, 150, 200, 300, 400 or 500 ng/ml, or one of any concentrations there between. In some embodiments, the concentration of BMP4 in the gonadal induction medium is about any one of: 0.5, 1, 2, 3, 4, 5, 6, 7, 8, 9, 10, 11, 12, 13, 14, 15, 16, 17, 18, 19, 20, or any concentrations there between. In some embodiments, the concentration of BMP4 in the gonadal induction medium is about any one of: 30, 32, 34, 36, 38, 40, 42, 44, 46, 48, 50, 52, 54, 56, 58, 60, 62, 64, 66, 68 or 70 ng/mL, or one of any concentrations there between. In some embodiments, the concentration of BMP4 in the gonadal induction medium is about 5 ng/ml to about 20 ng/mL. In some embodiments, the concentration of BMP4 in the gonadal induction medium is about 30 ng/mL to about 70 ng/mL. In some embodiments, the concentration of BMP4 in the gonadal induction medium is about 10 ng/mL. In some embodiments, the concentration of BMP4 in the gonadal induction medium is about 50 ng/mL.

In some embodiments according to any one of the methods or cell populations described herein, the BMP in the gonadal induction medium comprises BMP4, BMP2, BMP7, BMP15, or any combinations thereof. In some embodiments, the concentration of BMP in the gondal induction medium is about any one of: 5, 10, 15, 20, 25, 30, 35, 40, 45, 50, 55, 60, 65, 70, 75, 80, 85, 90, 95, 100, 125, 150, 200, 300, 400 or 500 ng/mL, or one of any concentrations there between. In some embodiments, the concentration of BMP in the gonadal induction medium is about any one of: 0.5, 1, 2, 3, 4, 5, 6, 7, 8, 9, 10, 11, 12, 13, 14, 15, 16, 17, 18, 19, 20, or any concentrations there between. In some embodiments, the concentration of BMP in the gonadal induction medium is about any one of: 30, 32, 34, 36, 38, 40, 42, 44, 46, 48, 50, 52, 54, 56, 58, 60, 62, 64, 66, 68 or 70 ng/ml, or one of any concentrations there between. In some embodiments, the concentration of BMP in the gonadal induction mediumis about 5 ng/mL to about 20 ng/mL. In some embodiments, the concentration of BMP in the gonadal induction medium is about 30 ng/mL to about 70 ng/mL. In some embodiments, the concentration of BMP in the gonadal induction mediumis about 10 ng/mL. In some embodiments, the concentration of BMP in the gonadal induction medium is about 20 ng/mL. In some embodiments, the concentration of BMP in the gonadal induction medium is about 50 ng/mL.

In some embodiments according to any one of the methods or cell populations described herein, the FGF in the gonadal induction medium is one or more of: FGF2, FGF4 or FGF9. In some embodiments, the FGF in the gonadal induction medium is FGF2. In some embodiments, the gonadal induction medium comprises FGF2, wherein the concentration of FGF2 is about any one of: 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 50, 60, 70, 80, 90, 100 ng/mL, or one of any one concentrations there between. In some embodiments, the concentration of FGF2 in the gonadal induction medium is about 1 ng/mL to about 10 ng/mL. In some embodiments, the concentration of FGF2 in the gonadal induction medium is about 5 ng/ml to about 25 ng/mL. In some embodiments, the concentration of FGF2 in the gonadal induction medium is about 1 ng/mL to about 10 ng/mL. In some embodiments, the concentration of FGF2 in the gonadal induction medium is about 5 ng/mL to about 25 ng/ml. In some embodiments, the concentration of FGF2 in the gonadal induction medium is about 5 ng/ml. In some embodiments, the concentration of FGF2 in the gonadal induction medium is about 10 ng/mL.

In some embodiments, the FGF in the gonadal induction medium comprises: FGF2, FGF4, FGF9, FGF10, FGF16, FGF17, FGF18, FGF19, or any combinations thereof. In some embodiments, the concentration of FGF is about any one of: 0.1, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 35, 40, 50, 60, 70, 80, 90, 100 ng/ml, or one of any one concentrations there between. In some embodiments, the concentration of FGF in the gonadal induction medium is about 1 ng/mL to about 10 ng/ml. In some embodiments, the concentration of FGF in the gonadal induction medium is about 5 ng/mL to about 25 ng/mL. In some embodiments, the concentration of FGF in the gonadal induction medium is about 1 ng/ml to about 10 ng/ml. In some embodiments, the concentration of FGF in the gonadal induction medium is about 5 ng/ml to about 25 ng/mL. In some embodiments, the concentration of FGF in the gonadal induction medium is about 5 ng/mL. In some embodiments, the concentration of FGF in the gonadal induction medium is about 10 ng/mL.

In some embodiments according to any one of the methods or cell populations described herein, the gonadal medium further comprises an RAPM. In some embodiments, the RAPM in the gonadal induction medium is an RAR agonist. In some embodiments, the RAPM comprises retinoic acid (RA) and/or TTNPB. In some embodiments, the RAPM is RA. In some embodiments, the RAPM is TTNPB. In some embodiments, the concentration of RA in the gonadal induction medium is about any one of: 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 5.0, 6.0, 7.0, 8.0, 9.0, 10.0, 20.0, 30.0, 40.0, 50.0 μM, or one of any concentrations there between. In some embodiments, the concentration of TTNPB in the gonadal induction medium is about any one of: 0.005, 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 5.0, 6.0, 7.0, 8.0, 9.0, 10.0, 20.0 μM, or one of any concentrations there between. In some embodiments, the concentration of RA in the gonadal induction medium is about 0.5 μM to about 2 μM, and/or the concentration of TTNPB in the gonadal induction medium is about 0.2 μM to about 1 μM. In some embodiments, the concentration of RA in the gonadal induction medium is about 1 μM; and/or the concentration of TTNPB in the gonadal induction medium is about 0.5 μM.

In some embodiments according to any one of the methods or cell populations described herein, the gonadal induction medium further comprises an apoptosis inhibitor. In some embodiments, the apoptosis inhibitor in the gonadal induction medium is Y-27632. In some embodiments, the concentration of Y-27632 is about any one of: 0.5, 1, 2, 3, 4, 5, 6, 7, 8, 9, 10, 11, 12, 13, 14, 15, 16, 17, 18, 19, 20, 21, 22, 23, 24, 25, 30, 35, 40, 50, 60, 70, 80, 90, 100 ng/mL, or one of any concentrations there between. In some embodiments, the concentration of Y-27632 is about 5 μM to about 20 μM. In some embodiments, the concentration of Y-27632 is about 10 μM. In some embodiments, the apoptosis inhibitor in the gonadal induction medium comprises Chroman1, Emricasan, and Trans-ISRIB. In some embodiments, the concentration of Chroman1 is about 30 nM to about 70 nM, the concentration of Emricasan is about 2 μM to about 10 M, and/or the concentration of Trans-ISRIB is about 0.2 μM to about 2 μM. In some embodiments, the concentration of Chroman1 is about 50 nM, the concentration of Emricasan is about 5 μM, and/or the concentration of Trans-ISRIB is about 0.7 μM.

In some embodiments according to any one of the methods or cell populations described herein, the period of time for culturing in gonadal induction medium is about any one of: 3, 4, 5, 6, 7, 8, 9, 10, 11, 12, 13, 14, 15, 16, 17, 18, 19, 20, 21, 22, 23, 24, 25, 28, 30, 35, 42, 49, 56, 63, 70 days, or one of any lengths there between. In some embodiments, the period of time for culturing in gonadal induction medium is at least about any one of: 3, 4, 5, 6, 7, 8, 9, 10, 11, 12, 13, 14, 15, 16, 17, 18, 19, 20, 21, 22, 23, 24, 25, 28, 30, 35, 42, 49, 56, 63, 70 days. In some embodiments, the period of time for culturing in gonadal induction medium is about 5 days to about 21 days. In some embodiments, the period of time for culturing in gonadal induction medium is about 7 days to about 14 days.

In some embodiments, provided herein is a method of producing a gonadal cell population, the method comprising: (a) culturing pluripotent stem cells in a mesoderm induction medium comprising about 50 ng/mL Activin A, about 3 μM CHIR99021 for about 56 to about 72 hours thereby producing a first intermediate cell population wherein at least about 80% of the cells within express Brachyury; (b) culturing the first intermediate cell population in an intermediate mesoderm induction medium comprising about 1 μM RA, about 3 μM CHIR99021, and about 20 ng/ml FGF2 for about 5 to 9 days, thereby producing a second intermediate cell population wherein at least about 80% of the cells within express PAX2; and (c) culturing the second intermediate cell population in a gonadal induction medium comprising about 25 ng/ml follistatin, about 10 ng/ml BMP4 and about 5 ng/ml FGF, for about 7 days to about 14 days, thereby producing the gonadal cell population wherein at least about 20% of the cells within express FOXL2 and/or NR2F2.

### Characterization of Gonadal Cell Populations and Intermediate Cell Populations

In some embodiments according to any one of the methods or cell populations described herein, the gonadal cell population can be characterized by one or more expression markers. In some embodiments, at least a portion of cells in the gonadal cell population express one or more of: FOXL2, NR2F2, WNT6, KITLG and NR1H4. In some embodiments, at least a portion of cells in the gonadal cell population express one or more of: NR2F2, GPC3 and COL1A1. In some embodiments, at least a portion of cells in the gonadal cell population express one or more of: NR2F2, GPC3 and COL1A1. In some embodiments, at least a portion of cells in the gonadal cell population express one or more of: KRT-19 and UPK3B. In some embodiments, at least a portion of the cells in the gonadal cell population express one or more of: FOXL2, NR2F2 and KRT-19.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express FOXL2. In some embodiments, at least about 20% of the cells within the gonadal cell population express FOXL2. In some embodiments, at least about 50% of the cells within the gonadal cell population express FOXL2.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express NR2F2. In some embodiments, at least about 20% of the cells within the gonadal cell population express NR2F2. In some embodiments, at least about 50% of the cells within the gonadal cell population express NR2F2.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express RUNX1. In some embodiments, at least about 20% of the cells within the gonadal cell population express RUNX1. In some embodiments, at least about 50% of the cells within the gonadal cell population express RUNX1.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express LGR5. In some embodiments, at least about 20% of the cells within the gonadal cell population express LGR5. In some embodiments, at least about 50% of the cells within the gonadal cell population express LGR5.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express WNT6. In some embodiments, at least about 20% of the cells within the gonadal cell population express WNT6. In some embodiments, at least about 50% of the cells within the gonadal cell population express WNT6.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express KITLG. In some embodiments, at least about 20% of the cells within the gonadal cell population express KITLG. In some embodiments, at least about 50% of the cells within the gonadal cell population express KITLG.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express NR1H4. In some embodiments, at least about 20% of the cells within the gonadal cell population express NR1H4. In some embodiments, at least about 50% of the cells within the gonadal cell population express NR1H4.

Granulosa cells become steroidogenic upon maturation—these cells are able to convert androgens such as testosterone into estrogens such as estradiol. One of the key enzymes responsible for steroidogenesis is Aromatase-CYP19A1.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express CYP19A1. In some embodiments, at least about 20% of the cells within the gonadal cell population express CYP19A1. In some embodiments, at least about 50% of the cells within the gonadal cell population express CYP19A1.

In some embodiments, the gonadal cell population secretes estradiol upon treatment with dihydroxy-testosterone (dhT). In some embodiments, the estradiol secretion level of a mature gonadal cell population upon treatment with dhT is at least about any one of: 10%, 20%, 50%, 80%, 100%, 10-fold, 20-fold, 50-fold, 100-fold, 1000-fold, 10000-fold, 100000-fold, or 1000000-fold higher than that of untreated cells. In some embodiments, the estradiol secretion level of a mature gonadal cell population upon treatment with dhT is at least about any one of: 10%, 20%, 50%, 80%, 100%, 10-fold, 20-fold, 50-fold, 100-fold, 1000-fold. 10000-fold, 100000-fold, or 1000000-fold higher than that of a bipotential gonadal cell population. In some embodiments, the estradiol secretion level of a mature gonadal cell population upon treatment with dhT is at least about any one of: 10%, 20%, 50%, 80%, 100%, 10-fold, 20-fold, 50-fold, 100-fold, 1000-fold, 10000-fold, 100000-fold, or 1000000-fold higher than that of an intermediate cell population expressing mesodermal markers. In some embodiments, the estradiol secretion level of a mature gonadal cell population upon treatment with dhT is at least about any one of: 10%, 20%, 50%, 80%, 100%, 10-fold, 20-fold, 50-fold, 100-fold, 1000-fold, 10000-fold, 100000-fold, or 1000000-fold higher than that of an intermediate cell population expressing intermediate mesodermal markers. In some embodiments, the estradiol secretion level of a mature gonadal cell population upon treatment with dhT is at least about any one of: 10%, 20%, 50%, 80%, 100%, 10-fold, 20-fold, 50-fold, 100-fold, 1000-fold, 10000-fold, 100000-fold, or 1000000-fold higher than that of pluripotent stem cells.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express GPC3. In some embodiments, at least about 20% of the cells within the gonadal cell population express GPC3. In some embodiments, at least about 50% of the cells within the gonadal cell population express GPC3.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express COL1A1. In some embodiments, at least about 20% of the cells within the gonadal cell population express COL1A1. In some embodiments, at least about 50% of the cells within the gonadal cell population express COL1A1.

In some embodiments, at least about any one of: 10%, 20%, 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first gonadal cell population express KRT19. In some embodiments, at least about 20% of the cells within the gonadal cell population express KRT19. In some embodiments, at least about 50% of the cells within the gonadal cell population express KRT19.

In some embodiments, at least 90% of the cells within the gonadal cell population are FOXL2-positive cells, NR2F2-positive cells, and/or KRT-19 positive cells. In some embodiments, the gonadal cell population consists essentially of FOXL2-positive cells, NR2F2-positive cells, and/or KRT-19 positive cells. In some embodiments, the FOXL2-positive cells comprise granulosa cells. In some embodiments, the NR2F2-positive cells comprise stroma cells and/or granulosa cells. In some embodiments, the KRT-19 positive cells comprise ovarian epithelial cells.

In some embodiments, the gonadal cell population comprises ovarian somatic cells (OSCs). In some embodiments, the gonadal cell population is an ovarian somatic cell population. In some embodiments, the gonadal cell population comprises bipotential gonadal somatic cells (such as bipotential ovarian somatic cells). In some embodiments, the bipotential gonadal somatic cells express one or more of: GATA4, WT1. LHX9 and ZFPM2. In some embodiments, the gonadal cell population comprises mature ovarian somatic cells. In some embodiments, the gonadal cell population comprises one or more of: granulosa cells, ovarian stromal cells and/or ovarian epithelial cells. In some embodiments, the granulosa cells express one or more of: FOXL2, KITLG and/or NR1H4. In some embodiments, the ovarian stromal cells express NR2F2. In some embodiments, the ovarian epithelial express one or more of: KRT19, MSLN, TMEM151A and/or LRRN4.

In some embodiments according to any one of the methods or cell populations described herein, the first intermediate cell population can be characterized by one or more expression markers. In some embodiments, at least a portion of cells in the first intermediate cell population express one or more of: Brachyury. MIXL1, N-Cadherin, ECPAM, NCAM.

In some embodiments, at least a portion of the cells in the first intermediate cell population express Brachyury. In some embodiments, at least a portion of the cells in the first intermediate cell population express MIXL1. In some embodiments, at least a portion of the cells in the first intermediate cell population express N-Cadherin. In some embodiments, at least a portion of the cells in the first intermediate cell population express EpCam. In some embodiments, at least a portion of the cells in the first intermediate cell population express NCAM. In some embodiments, at least a portion of the cells in the first intermediate cell population express GSC. In some embodiments, at least a portion of the cells in the first intermediate cell population express two or more of: Brachyury, MIXL1, N-Cadherin, EpCam, NCAM. In some embodiments, at least a portion of the cells in the first intermediate cell population express three or more of: Brachyury, MIXL1, N-Cadherin, EpCam, NCAM.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first intermediate cell population express Brachyury. In some embodiments, at least about 80% of the cells within the first intermediate cell population express Brachyury. In some embodiments, at least about 90% of the cells within the first intermediate cell population express Brachyury. In some embodiments, at least about 95% of the cells within the first intermediate cell population express Brachyury.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first intermediate cell population express MIXL1. In some embodiments, at least about 80% of the cells within the first intermediate cell population express MIXL1. In some embodiments, at least about 90% of the cells within the first intermediate cell population express MIXL1.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first intermediate cell population express N-Cadherin. In some embodiments, at least about 80% of the cells within the first intermediate cell population express N-Cadherin. In some embodiments, at least about 90% of the cells within the first intermediate cell population express N-Cadherin.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first intermediate cell population express EPCAM. In some embodiments, at least about 80% of the cells within the first intermediate cell population express EPCAM. In some embodiments, at least about 90% of the cells within the first intermediate cell population express EPCAM.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first intermediate cell population express N-Cadherin. In some embodiments, at least about 80% of the cells within the first intermediate cell population express N-Cadherin. In some embodiments, at least about 90% of the cells within the first intermediate cell population express N-Cadherin.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first intermediate cell population express NCAM. In some embodiments, at least about 80% of the cells within the first intermediate cell population express NCAM. In some embodiments, at least about 90% of the cells within the first intermediate cell population express NCAM.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first intermediate cell population express GSC. In some embodiments, at least about 80% of the cells within the first intermediate cell population express GSC. In some embodiments, at least about 90% of the cells within the first intermediate cell population express GSC.

In some embodiments, at least about any one of: 30%, 40%, 50%, 60%, 70%, 96%, 97%, or 98% of the cells within the first intermediate cell population are mesodermal cells. In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the first intermediate cell population are mesoderm-like cells. In some embodiments, a mesoderm-like cell can refer to a cell displaying one or more markers of a corresponding mesoderm in vivo. In some embodiments, a mesoderm-like cell can refer to a cell having some or all of the developmental potential of a corresponding mesoderm in vivo. In some embodiments, at least about 90% of the cells within the first intermediate cell population are mesodermal cells. In some embodiments, at least about 90% of the cells within the first intermediate cell population are mesoderm-like cells. In some embodiments, at least at least about 90% of the cells within the first intermediate cell population are mesodermal cells or mesoderm-like cells.

In some embodiments according to any one of the methods or cell populations described herein, the second intermediate cell population can be characterized by one or more expression markers. In some embodiments, at least a portion of the cells in the second intermediate cell population express one or more of: OSR1, PAX2, LHX1, and RUNX1.

In some embodiments, at least a portion of the cells in the second intermediate cell population express OSR1. In some embodiments, at least a portion of the cells in the second intermediate cell population express PAX2. In some embodiments, at least a portion of the cells in the second intermediate cell population express LHX1. In some embodiments, at least a portion of the cells in the second intermediate cell population express WT1. In some embodiments, at least a portion of the cells in the second intermediate cell population express SALL1. In some embodiments, at least a portion of the cells in the second intermediate cell population express GSC. In some embodiments, at least a portion of the cells in the second intermediate cell population express one or more of: OSR1, PAX2, LHX1, RUNX1, WT1 and SALL1. In some embodiments, at least a portion of the cells in the second intermediate cell population express two or more of: OSR1, PAX2, LHX1, RUNX1, WT1 and SALL1. In some embodiments, at least a portion of the cells in the second intermediate cell population express three or more of: OSR1, PAX2, LHX1. RUNX1, WT1 and SALL1.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the second intermediate cell population express LHX1. In some embodiments, at least about 90% of the cells within the second intermediate cell population express LHX1. In some embodiments, at least about 95% of the cells within the second intermediate cell population express LHX1.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the second intermediate cell population express PAX2. In some embodiments, at least about 90% of the cells within the second intermediate cell population express PAX2. In some embodiments, at least about 95% of the cells within the second intermediate cell population express PAX2.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the second intermediate cell population express OSR1. In some embodiments, at least about 80% of the cells within the second intermediate cell population express OSR1. In some embodiments, at least about 90% of the cells within the second intermediate cell population express OSR1.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the second intermediate cell population express RUNX1. In some embodiments, at least about 80% of the cells within the second intermediate cell population express RUNX1. In some embodiments, at least about 90% of the cells within the second intermediate cell population express RUNX1.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the second intermediate cell population express WT1. In some embodiments, at least about 80% of the cells within the second intermediate cell population express WT1. In some embodiments, at least about 90% of the cells within the second intermediate cell population express WT1.

In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the second intermediate cell population express SALL1. In some embodiments, at least about 80% of the cells within the second intermediate cell population express SALL1. In some embodiments, at least about 90% of the cells within the second intermediate cell population express SALL1.

In some embodiments, at least about any one of: 30%, 40%, 50%, 60%, 70%, 96%, 97%, or 98% of the cells within the second intermediate cell population are intermediate mesodermal cells. In some embodiments, at least about any one of: 30%, 40%, 50%, 60%, 70%, 80%, 81%, 82%, 83%, 84%, 85%, 86%, 87%, 88%, 89%, 90%, 91%, 92%, 93%, 94%, 95%, 96%, 97%, or 98% of the cells within the second intermediate cell population are intermediate mesoderm-like cells. In some embodiments, an intermediate mesoderm-like cell can refer to a cell displaying one or more markers of a corresponding intermediate mesoderm in vivo. In some embodiments, a mesoderm-like cell can refer to a cell having some or all of the developmental potential of a corresponding intermediate mesoderm in vivo. In some embodiments, at least about 80% of the cells within the second intermediate cell population are intermediate mesodermal cells. In some embodiments, at least about 90% of the cells within the second intermediate cell population are intermediate mesoderm-like cells. In some embodiments, at least 90% of the cells within the second intermediate cell population are intermediate mesodermal cells and/or intermediate mesoderm-like cells.

In some embodiments, (I) the potential of the second intermediate cell population to differentiate into NR2F2-expressing gonadal cells is increased; and/or (II) the potential of the second intermediate cell population to differentiate into FOXL2-expressing gonadal cells is increased; and/or (III) the potential of the second intermediate cell population to differentiate into RUNX1-expressing gonadal cells is increased; and/or (IV) the potential of the second intermediate cell population to differentiate into WNT6-expressing gonadal cells is increased; and/or (V) the potential of the second intermediate cell population to differentiate into NR5A1-expressing gonadal cells is increased; and/or (VI) the potential of the second intermediate cell population to differentiate into OSR1-expressing gonadal cells is increased; and/or (VII) the potential of the second intermediate cell population to differentiate into DLK1- and/or GPC3-expressing gonadal cells is increased; and/or (VIII) the potential of the second intermediate cell population to differentiate into LHX9-expressing gonadal cells is increased; and/or (IX) the potential of the second intermediate cell population to differentiate into KRT19-expressing gonadal cells is increased; as compared to a corresponding second intermediate cell population generated by a method wherein the intermediate mesoderm induction medium does not comprise RAPM.

In some embodiments, (I) the potential of the second intermediate cell population to differentiate into NR2F2-expressing gonadal cells is increased; and/or (II) the potential of the second intermediate cell population to differentiate into FOXL2-expressing gonadal cells is increased; and/or (III) the potential of the second intermediate cell population to differentiate into RUNX1-expressing gonadal cells is increased; and/or (IV) the potential of the second intermediate cell population to differentiate into WNT6-expressing gonadal cells is increased; and/or (V) the potential of the second intermediate cell population to differentiate into NR5A1-expressing gonadal cells is increased; and/or (VI) the potential of the second intermediate cell population to differentiate into OSR1-expressing gonadal cells is increased; and/or (VII) the potential of the second intermediate cell population to differentiate into DLK1- and/or GPC3-expressing gonadal cells is increased; and/or (VIII) the potential of the second intermediate cell population to differentiate into LHX9-expressing gonadal cells is increased; and/or (IX) the potential of the second intermediate cell population to differentiate into KRT19-expressing gonadal cells is increased, as compared to a corresponding second intermediate cell population generated by a method wherein the intermediate mesoderm induction medium comprises a lower concentration of RAPM.

In some embodiments, (I) the potential of the second intermediate cell population to differentiate into NR2F2-expressing gonadal cells is increased; and/or (II) the potential of the second intermediate cell population to differentiate into FOXL2-expressing gonadal cells is increased; and/or (III) the potential of the second intermediate cell population to differentiate into RUNX1-expressing gonadal cells is increased; and/or (IV) the potential of the second intermediate cell population to differentiate into WNT6-expressing gonadal cells is increased; and/or (V) the potential of the second intermediate cell population to differentiate into NR5A1-expressing gonadal cells is increased; and/or (VI) the potential of the second intermediate cell population to differentiate into OSR1-expressing gonadal cells is increased; and/or (VII) the potential of the second intermediate cell population to differentiate into DLK1- and/or GPC3-expressing gonadal cells is increased; and/or (VIII) the potential of the second intermediate cell population to differentiate into LHX9-expressing gonadal cells is increased; and/or (IX) the potential of the second intermediate cell population to differentiate into KRT19-expressing gonadal cells is increased, as compared to a corresponding second intermediate cell population generated by a method wherein the intermediate mesoderm induction medium does not comprise glycogen synthase kinase-3 inhibitor.

In some embodiments, (I) the potential of the second intermediate cell population to differentiate into NR2F2-expressing gonadal cells is increased; and/or (II) the potential of the second intermediate cell population to differentiate into FOXL2-expressing gonadal cells is increased; and/or (III) the potential of the second intermediate cell population to differentiate into RUNX1-expressing gonadal cells is increased; and/or (IV) the potential of the second intermediate cell population to differentiate into WNT6-expressing gonadal cells is increased; and/or (V) the potential of the second intermediate cell population to differentiate into NR5A1-expressing gonadal cells is increased; and/or (VI) the potential of the second intermediate cell population to differentiate into OSR1-expressing gonadal cells is increased; and/or (VII) the potential of the second intermediate cell population to differentiate into DLK1- and/or GPC3-expressing gonadal cells is increased; and/or (VIII) the potential of the second intermediate cell population to differentiate into LHX9-expressing gonadal cells is increased; and/or (IX) the potential of the second intermediate cell population to differentiate into KRT19-expressing gonadal cells is increased, as compared to a corresponding second intermediate cell population generated by a method wherein the intermediate mesoderm induction medium comprises a lower concentration of glycogen synthase kinase-3 inhibitor.

In some embodiments, the concentration of and exposure duration to RAPM can be adjusted in the derivation of the gonadal somatic cell population. In some embodiments, the cell fate and composition of the gonadal somatic cell population generated thereby is rendered different when the concentration of and exposure duration to RAPM is adjusted during derivation. In some embodiments, the RAPM comprises retinoic acid (RA) and/or TTNPB. In some embodiments, the RAPM is RA. In some embodiments, the RAPM is TTNPB.

In some embodiments, the RAPM is RA. In some embodiments according to any of the methods or cell populations described herein, the gonadal induction medium comprises RA at a concentration of at least about any one of: 0.01, 0.05, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 3.0, 3.5, 4.0, 5.0, 6.0, 7.0, 8.0, 9.0, 10.0, 20.0, 30.0, 40.0, 50.0 μM, or one of any concentrations there between. In some embodiments, the gonadal induction is conducted in the presence of RA (at any one of the concentration described above, such as but not limited to 0.1, 0.5, or 1.0 μM) for at least about any one of 2, 4, 6, 8, 10, 12, 16, 20, 24, 28, 32, 36, 40, 44, 48, 72, 96, 120, 144, 168, 192, or 240 hours.

In some embodiments according to any of the methods or cell populations described herein, (I) the amount of FOXL2 expression in the gonadal cell population is decreased; and/or (II) the amount of expression of NR1H4 and/or KITLG in the gonadal cell population is decreased; and/or (III) the amount of KRT-19 expression in the gonadal cell population is increased; and/or (IV) the amount of cytoplasmic KRT-19 expression in the gonadal cell population is increased; and/or (V) the amount of expression of MSLN, LRRN4, and/or TMEM151A in the gonadal cell population is increased, as compared to a corresponding gonadal cell population generated by a method wherein the gonadal induction medium comprises a lower concentration of RAPM.

In some embodiments according to any of the methods or cell populations described herein, (I) the amount of FOXL2 expression in the gonadal cell population is decreased; and/or (II) the amount of expression of NR1H4 and/or KITLG in the gonadal cell population is decreased; and/or (III) the amount of KRT-19 expression in the gonadal cell population is increased; and/or (IV) the amount of cytoplasmic KRT-19 expression in the gonadal cell population is increased; and/or (V) the amount of expression of MSLN, LRRN4, and/or TMEM151A in the gonadal cell population is increased, as compared to a corresponding gonadal cell population generated by a method wherein the gonadal induction medium does not comprise a RAPM.

In some embodiments according to any of the methods or cell populations described herein, (I) the amount of FOXL2 expression in the gonadal cell population is decreased; and/or (II) the amount of expression of NR1H4 and/or KITLG in the gonadal cell population is decreased; and/or (III) the amount of KRT-19 expression in the gonadal cell population is increased; and/or (IV) the amount of cytoplasmic KRT-19 expression in the gonadal cell population is increased; and/or (V) the amount of expression of MSLN, LRRN4, and/or TMEM151A in the gonadal cell population is increased, as compared to a corresponding gonadal cell population generated by a gonadal induction step comprising contacting with an RAPM for a shorter period of time.

Also provided is a gonadal cell population (e.g. ovarian somatic cells) produced by any of the provided methods.

Also provided herein is an in vitro stem cell-derived gonadal somatic cell population comprising FOXL2-expressing cells, NR2F2-expressing cells and/or KRT-19 expressing cells. In some embodiments, the gonadal somatic cell population is an ovarian somatic cell population. In some embodiments, the population comprises at least a first cell type expressing FOXL2, a second cell type expressing NR2F2, and a third cell type expressing KRT-19. In some embodiments, at least 20% of cells within the cell population are FOXL2-positive cells, such as at least 25%, at least 30%, at least 40% or at least 50% of the cells within the population are FOXL2-positive cells. In some embodiments, at least 20% of the cells within the cell population are NR2F2-positive cells, such as at least 25%, at least 30%, at least 40% or at least 50% of the cells within the population are NR2F2-positive cells. In some embodiments, at least 20% of the cells within the gonadal cell population are KRT19-positive cells, such as at least 25%, at least 30%, at least 40% or at least 50% of the cells within the population are KRT192-positive cells. In some embodiments, (a) at least 20% of cells within the cell population are FOXL2-positive cells, and/or (b) at least 20% of the cells within the cell population are NR2F2-positive cells; and/or (c) at least 20% of the cells within the gonadal cell population are KRT19-positive cells. In some embodiments, (a) about 20%-40% of cells within the cell population are FOXL2-positive cells, and/or (b) about 20%-40% of the cells within the cell population are NR2F2-positive cells; and/or (c) about 20%-40% of the cells within the gonadal cell population are KRT19-positive cells. In some embodiments, least 85% of the gonadal cell population are FOXL2-positive cells, NR2F2-positive cells, and/or KRT-19 positive cells. In some embodiments, least 90% of the gonadal cell population are FOXL2-positive cells. NR2F2-positive cells, and/or KRT-19 positive cells. In some embodiments, least 95% of the gonadal cell population are FOXL2-positive cells, NR2F2-positive cells, and/or KRT-19 positive cells. In some embodiments, the gonadal somatic cell population consists essentially of FOXL2-positive cells. NR2F2-positive cells, and/or KRT-19 positive cells. In some embodiments, the FOXL2-positive cells comprise granulosa cells; the NR2F2-positive cells comprise ovarian stromal cells and/or granulosa cells; and the KRT-19 positive cells comprise ovarian epithelial cells.

In some of any embodiments of a provided gonadal somatic cell population the cell population is differentiated from pluripotent stem cells. In some embodiments, the gonadal somatic cell population (e.g. ovarian somatic cells) can be produced by any of the provided methods herein. In some of any embodiments, the gonadal somatic cell population is derived in a process comprising: (a) culturing pluripotent stem cells in a mesoderm induction medium comprising Activin A for a first period of time to produce a first intermediate cell population; (b) culturing the first intermediate cell population in an intermediate mesoderm induction medium comprising an retinoic acid pathway modulator (RAPM) for a second period of time, thereby producing a second intermediate cell population; and (c) culturing the second intermediate cell population in a gonadal induction medium comprising BMP and FGF, and optionally follistatin, for a third period of time to produce the gonadal cell population.

In some of any embodiments, the gonadal somatic cell population is or has been cryopreserved. In some embodiments, there is provided a composition comprising a gonadal somatic cell population. In some embodiments, the composition further comprises a cryoprotectant. In some of any of the provided embodiments, the method further includes formulating the gonadal cell population, such as a cell population produced or harvested by the provided methods, with a cryoprotectant (also called a cryopreservative). In some embodiments, the cryoprotectant is selected from glycerol, propylene glycol, dimethyl sulfoxide (DMSO), or a combination thereof. In some embodiments, the cryoprotectant includes DMSO. In some embodiments, the cryoprotectant is DMSO.

In some embodiments, the formulation buffer contains a cryopreservative. In some embodiments, the cells are formulated with a cyropreservative solution that contains 1.0% to 30% DMSO solution, such as a 5% to 20% DMSO solution or a 5% to 10% DMSO solution. In some embodiments, the cryopreservation solution is or contains, for example, PBS containing 20% DMSO and 8% human serum albumin (HSA), or other suitable cell freezing media. In some embodiments, the cryopreservative solution is or contains, for example, at least or about 7.5% DMSO. In some embodiments, the method includes one or more processing steps that can involve washing the differentiated cells to replace the cells in a cryopreservative solution. In some embodiments, the cells are frozen, e.g., cryopreserved or cryoprotected, in media and/or solution with a final concentration of or of about 12.5%. 12.0%, 11.5%, 11.0%, 10.5%, 10.0%, 9.5%. 9.0%, 8.5%, 8.0%, 7.5%, 7.0%, 6.5%, 6.0%. 5.5%, or 5.0% DMSO, or between 1% and 15%, between 6% and 12%, between 5% and 10%, or between 6% and 8% DMSO. In particular embodiments, the cells are frozen, e.g., cryopreserved or cryoprotected, in media and/or solution with a final concentration of or of about 5.0%, 4.5%, 4.0%, 3.5%, 3.0%, 2.5%, 2.0%, 1.5%, 1.25%, 1.0%, 0.75%, 0.5%, or 0.25% HSA, or between 0.1% and −5%, between 0.25% and 4%, between 0.5% and 2%, or between 1% and 2% HSA. In some embodiments, the gonadal cell population (e.g. ovarian somatic cells) produced by the method are formulated with about 10% DMSO. In some embodiments, the one or more compositions have been previously cryopreserved and stored, and are thawed prior to their further use.

### Exemplary Embodiments

Among the provided embodiments are:
1. A method of producing a gonadal cell population, the method comprising:
(a) culturing pluripotent stem cells in a mesoderm induction medium comprising Activin A for a first period of time to produce a first intermediate cell population;
(b) culturing the first intermediate cell population in an intermediate mesoderm induction medium comprising an retinoic acid pathway modulator (RAPM) for a second period of time, thereby producing a second intermediate cell population; and
(c) culturing the second intermediate cell population in a gonadal induction medium comprising BMP and FGF, for a third period of time to produce the gonadal cell population.
2. A method of producing a gonadal cell population, the method comprising:
(a) culturing pluripotent stem cells in a mesoderm induction medium comprising Activin A for a first period of time to produce a first intermediate cell population;
(b) culturing the first intermediate cell population in an intermediate mesoderm induction medium comprising an retinoic acid pathway modulator (RAPM) for a second period of time, thereby producing a second intermediate cell population; and
(c) culturing the second intermediate cell population in a gonadal induction medium comprising follistatin, BMP4 and FGF, for a third period of time to produce the gonadal cell population.
3. A method of producing a gonadal cell population, the method comprising:
(a) culturing mesoderm or mesoderm-like cells in an intermediate mesoderm induction medium comprising a retinoic acid pathway modulator (RAPM) for a first period of time thereby producing a second intermediate cell population; and
(b) culturing the second intermediate cell population in a gonadal induction medium comprising BMP, and FGF for a second period of time to produce the gonadal cell population
4. A method of producing a gonadal cell population, the method comprising:
(a) culturing mesoderm or mesoderm-like cells in an intermediate mesoderm induction medium comprising a retinoic acid pathway modulator (RAPM) for a first period of time thereby producing a second intermediate cell population; and
(b) culturing the second intermediate cell population in a gonadal induction medium comprising follistatin, BMP4, and FGF for a second period of time to produce the gonadal cell population.
5. A method of producing a gonadal cell population, the method comprising:
culturing intermediate mesoderm or intermediate mesoderm-like cells in a gonadal induction medium comprising. BMP and FGF for a period of time to produce the gonadal cell population.
6. A method of producing a gonadal cell population, the method comprising:
culturing intermediate mesoderm or intermediate mesoderm-like cells in a gonadal induction medium comprising follistatin, BMP4 and FGF for a period of time to produce the gonadal cell population.
7. A method of producing a first intermediate cell population, the method comprising: culturing pluripotent stem cells in a mesoderm induction medium comprising Activin A and a glycogen synthase kinase-3 inhibitor for a period of time to produce a first intermediate cell population
8. A method of producing a second intermediate cell population, the method comprising:
culturing mesoderm or mesoderm-like cells in an intermediate mesoderm induction medium comprising a retinoic acid pathway modulator (RAPM) for a period of time, thereby producing the second intermediate cell population.
9. A method of producing a second intermediate cell population, the method comprising:
(a) culturing pluripotent stem cells in a mesoderm induction medium comprising activin A and a glycogen synthase kinase-3 inhibitor for a first period of time to produce a first intermediate cell population; and
(b) culturing the first intermediate cell population in an intermediate mesoderm induction medium comprising a retinoic acid pathway modulator (RAPM), FGF, and a glycogen synthase kinase-3 inhibitor for a second period of time, thereby producing the second intermediate cell population.
10. The method of any one of embodiments 1-4 and 9, wherein at least a portion of cells in the first intermediate cell population express Brachyury.
11. The method of any one of embodiments 1-4, 8, and 9, wherein at least a portion of the cells in the second intermediate cell population express OSR1, PAX2, or LHX
12. The method of any one of embodiments 1-6, wherein at least a portion of the cells in the gonadal cell population express FOXL2, NR2F2, or RUNX1
13. The method of any one of embodiments 1, 2, 7, and 9-11, wherein the pluripotent stem cells are seeded at a density of about 10,000 to about 40,000 cells per cm².
14. The method of any one of embodiments 1, 2, 7, and 9-13, wherein the pluripotent stem cells are seeded in a culture plate coated with fibronectin.
15. The method of any one of embodiments 1, 2, 7, and 9-13, wherein the pluripotent stem cells are seeded in a culture plate coated with matrigel.
16. The method of any one of embodiments 1-15, wherein the mesoderm induction medium further comprises FGF.
17. The method of any one of embodiments 1-16, wherein the mesoderm induction medium further comprises BMP4.
18. The method of any one of embodiments 1-17, wherein the mesoderm induction medium further comprises a glycogen synthase kinase-3 inhibitor.
19. The method of any one of embodiments 1-18, wherein the mesoderm induction medium further comprises an apoptosis inhibitor.
20. The method any one of embodiments 1-19, wherein the concentration of Activin A in the mesoderm induction medium is about 30 ng/ml to about 70 ng/ml.
21. The method of embodiment 20, wherein the concentration of Activin A in the mesoderm induction medium is about 50 ng/mL.
22. The method of any one of embodiments 16-21, wherein the FGF in the mesoderm induction medium is FGF2.
23. The method of embodiment 22, wherein the concentration of FGF2 in the mesoderm induction medium is about 5 ng/mL to about 20 ng/mL, optionally wherein the concentration of FGF2 in the mesoderm induction medium is about 12 ng/ml.
24. The method of any one of embodiments 17-23, wherein the concentration of BMP4 in the mesoderm induction medium is about 10 ng/ml to about 50 ng/ml, optionally wherein the concentration of BMP4 in the mesoderm induction medium is about 30 ng/ml.
25. The method any one of embodiments 18-24, wherein the glycogen synthase kinase-3 inhibitor in the mesoderm induction medium is CHIR99021.
26. The method of embodiment 25, wherein the concentration of CHIR99021 is about 1 μM to about 5 μM.
27. The method of embodiment 26, wherein the concentration of CHIR99021 in the mesoderm induction medium is about 3 μM.
28. The method of any one of embodiments 19-27, wherein within the mesoderm-induction medium:
(A) the apoptosis inhibitor is Y-27632, optionally wherein the concentration of Y-27632 is about 5 μM to about 20 μM; or
(B) the apoptosis inhibitor comprises Chroman1, Emricasan, and Trans-ISRIB, optionally wherein the concentration of Chroman1 is about 30 nM to about 70 nM, the concentration of Emricasan is about 2 μM to about 10 μM, and the concentration of Trans-ISRIB is about 0.2 μM to about 2 μM.
29. The method of embodiment 28, wherein within the mesoderm induction media:
(A) the concentration of Y-27632 is about 10 μM; or
(B) the concentration of Chroman1 is about 50 nM, the concentration of Emricasan is about 5 μM, and the concentration of Trans-ISRIB is about 0.7 μM.
30. The method of any one of embodiments 1-29, wherein the period of time for culturing in mesoderm induction medium is about 24 hours to about 96 hours.
31. The method of embodiment 30, wherein the period of time for culturing in mesoderm induction medium is about 24 hours to about 72 hours.
32. The method of embodiment 31, wherein the period of time for culturing in mesoderm induction medium is about 56 hours to about 72 hours
33. The method of embodiment any one of embodiments 1-32, wherein at least 90% of cells in the first intermediate cell population express Brachyury.
34. The method of embodiment any one of embodiments 1-33, wherein at least 80% of cells in the first intermediate cell population express one or more of: MIXL1, N-Cadherin, EpCam, NCAM.
35. The method of embodiment 1-34, wherein at least 90% of cells in the first intermediate cell population expresses: Brachyury, N-Cadherin, EpCam, and NCAM
36. The method of embodiment any one of embodiments 1-35, wherein at least 90% of cells in the first intermediate cell population are mesoderm or mesoderm-like cells.
37. The method of embodiment 36, wherein the first intermediate cell population consists essentially of mesoderm or mesoderm-like cells.
38. The method of any one of embodiments 1-37, wherein the first intermediate cell population is replated onto a new fibronectin-coated culture plate prior to culturing in intermediate mesoderm induction medium; optionally wherein the first intermediate cell population is enzymatically detached, centrifuged and resuspended before replating.
39. The method of embodiment 38, wherein the first intermediate cell population is plated a density of about 5,000 to about 25,000 cells/cm².
40. The method of any one of embodiments 1-39, wherein the intermediate mesoderm induction medium further comprises an FGF.
41. The method of any one of embodiments 1-40, wherein the intermediate mesoderm induction medium further comprises a glycogen synthase kinase-3 inhibitor.
42. The method of any one of embodiments 1-41, wherein the intermediate mesoderm induction medium further comprises Activin A.
43. The method of any one of embodiments 1-42, wherein the intermediate mesoderm induction medium further comprises an apoptosis inhibitor.
44. The method of embodiment 43, wherein the method comprises culturing the first intermediate cell population
(i) first in the intermediate mesoderm induction medium with a first concentration of the apoptosis inhibitor,
(ii) subsequently in the intermediate mesoderm induction medium with no more than a second concentration of the apoptosis inhibitor.
45. The method of any one of embodiments 1-44, wherein the RAPM in the intermediate mesoderm induction medium is an RAR agonist, optionally wherein the RAPM comprises retionic acid (RA) and/or TTNPB.
46. The method of any one of embodiments 1-45, wherein:
   (a) the RAPM is RA, wherein the concentration of RA in the intermediate mesoderm induction medium is about 0.5 μM to about 2 μM; and/or
   (b) the RAPM is TTNPB, wherein the concentration of TTNPB in the intermediate mesoderm induction medium is about 0.2 M to about 1 μM.
47. The method of embodiment 46, wherein:
   (a) the concentration of RA in the intermediate mesoderm induction medium is about 1 μM; and/or
   (b) the concentration of TTNPB in the intermediate mesoderm induction medium is about 0.5 μM.
48. The method of any one of embodiments 40-47, wherein the FGF in the intermediate mesoderm induction medium is FGF2, wherein the concentration of the FGF2 is about 10 ng/ml to about 30 ng/mL.
49. The method of embodiment 48, wherein the concentration of the FGF2 in the intermediate mesoderm induction medium is about 20 ng/ml.
50. The method of any one of embodiments 41-49, wherein the glycogen synthase kinase-3 inhibitor in the intermediate mesoderm induction medium is CHIR99021, wherein the concentration of CHIR99021 is about 1 μM to about 5 μM.
51. The method of embodiment 50, wherein the concentration of CHIR99021 in the intermediate mesoderm induction medium is about 2 μM or about 3 μM.
52. The method of any one of embodiments 42-51, wherein the concentration of Activin A in the intermediate mesoderm induction medium is about 10 ng/mL to about 50 ng/ml.
53. The method of embodiment 52, wherein the concentration of Activin A in the intermediate mesoderm induction medium is about 30 ng/ml
54. The method of any one of embodiments 43 and 46-53, wherein within the mesoderm-induction medium:
(A) the apoptosis inhibitor is Y-27632, wherein the concentration of Y-27632 is about 5 μM to about 20 μM; or
(B) the apoptosis inhibitor comprises Chroman1, Emricasan, and Trans-ISRIB, wherein the concentration of Chroman1 is about 30 nM to about 70 nM, the concentration of Emricasan is about 2 μM to about 10 μM, and the concentration of Trans-ISRIB is about 0.2 μM to about 2 μM.
55. The method of any one of embodiments 45-54, wherein the apoptosis inhibitor in the intermediate mesoderm induction medium is Y-27632, wherein the method comprises culturing the first intermediate cell population:
(i) first in the intermediate mesoderm induction medium comprising about 10 μM of Y-27632; and
(ii) subsequently in the intermediate mesoderm induction medium with no more than about 2 μM of Y-27632.
56. The method of any one of embodiments 45-55, wherein the method comprises culturing the first intermediate cell population:
(i) first in the intermediate mesoderm induction medium comprising about 10 μM of Y-27632 for about 24 hours,
(ii) subsequently in the intermediate mesoderm induction medium with no more than about 2 μM of Y-27632 for about 5-6 days.
57. The method of any one of embodiments 1-56, wherein the period of time for culturing in the intermediate mesoderm induction medium is about 4-14 days.
58. The method of any one of embodiments 1-57, wherein the period of time for culturing in the intermediate mesoderm induction medium is about 5-9 days.
59. The method of any one of embodiment 1-58, wherein the apoptosis inhibitor in the intermediate mesoderm induction medium is Y-27632, wherein the concentration of Y-27632 is:
(a) about 10 μM during the first 24 hours:
(b) no more than about 2 μM between 24 hours and 72 hours;
(c) no more than about 0.5 μM between 72 hours and 120 hours; or
(d) no more than about 0.1 μM after 120 hours, during the culturing in the intermediate mesoderm induction medium.
60. The method of any one of embodiments 44-59, wherein the first intermediate cell population is cultured in intermediate mesoderm induction medium comprising an apoptosis inhibitor for about 24 hours, and wherein after about 24 hours, a portion of the medium is first replaced with an intermediate mesoderm induction medium not comprising the apoptosis inhibitor.
61. The method of embodiment 60, wherein portion of the medium is about 80% of the medium.
62. The method of embodiment 60 or 61, wherein the method further comprises subsequent medium replacements, wherein the medium replacements comprise replacing a portion of the medium every about 48 hours after the initial 24 hours of culturing.
63. The method of embodiment 62, wherein the portion of the medium in each subsequent medium replacement is about 80% of the medium.
64. The method of any one of embodiments 1-63, wherein at least 90% of cells in the second intermediate cell population expresses one or more of: OSR1. PAX2, LHX1, and RUNX1.
65. The method of any one of embodiments 1-64, wherein at least 90% of cells in the second intermediate cell population expresses two or more of: OSR1, PAX2, LHX1, and RUNX1.
66. The method of any one of embodiments 1-65, wherein at least 90% of the cells in the second intermediate cell population expresses OSR1, PAX2, and LHX1.
67. The method of any one of embodiments 1-66, wherein at least 90% of cells in the second intermediate cell population are intermediate mesoderm or intermediate mesoderm-like cells.
68. The method of embodiment 67, wherein the second intermediate cell population consists essentially of intermediate mesoderm or intermediate mesoderm-like cells.
69. The method of any one of embodiments 1-68, wherein the second intermediate cell population is replated onto a new fibronectin-coated culture plate prior to culturing in gonadal induction medium; optionally wherein the second intermediate cell population is enzymatically detached, centrifuged and resuspended before replating.
70. The method of embodiment 69, wherein the second intermediate cell population is plated at a density of about 5,000 to about 25,000 cells/cm².
71. The method of any one of embodiments 1-70, wherein the gonadal induction medium further comprises an RAPM.
72. The method of any one of embodiments 1-71, wherein the gonadal induction medium further comprises an apoptosis inhibitor.
73. The method of any one of embodiments 1, 3, 5 and 10-72, wherein the gonadal induction medium further comprises follistatin.
74. The method of any one of embodiments 1-73, wherein the concentration of follistatin in the gonadal induction medium is about 10 ng/mL to about 50 ng/mL.
75. The method of embodiment 74, wherein the concentration of follistatin in the gonadal induction medium about 25 ng/mL.
76. The method of any one of embodiments 1, 3, 5 and 10-75, wherein the BMP in the gonadal induction medium comprises BMP4, BMP2, BMP7, BMP15, or any combinations thereof.
77. The method of any one of embodiments 1, 3, 5 and 10-76, wherein the total concentration of BMP in the gonadal induction medium is about 5 ng/ml to about 20 ng/ml, or about 20 ng/ml to about 70 ng/ml.
78. The method of embodiment 77, wherein the total concentration of BMP in the gonadal induction medium is about 10 ng/ml or about 50 ng/mL.
79. The method of any one of embodiments 1-78, wherein the concentration of BMP4 in the gonadal induction medium is about 5 ng/mL to about 20 ng/mL, or about 20 ng/mL to about 70 ng/mL.
80. The method of embodiment 79, wherein the concentration of BMP4 in the gonadal induction medium is about 10 ng/ml or about 50 ng/mL.
81. The method of any one of embodiments 1-80, wherein the FGF in the gonadal induction medium comprises FGF2, FGF9, FGF10, FGF16, FGF17, FGF18, FGF19, or any combinations thereof.
82. The method of any one of embodiments 1-81, wherein the concentration of FGF is about 1 ng/ml to about 10 ng/ml or about 5 ng/ml to about 25 ng/mL.
83. The method of embodiment 82, wherein the concentration of FGF is about 5 ng/ml or about 10 ng/ml
84. The method of any one of embodiments 1-83, wherein the FGF in the gonadal induction medium is FGF2, wherein the concentration of the FGF2 is about 1 ng/mL to about 10 ng/ml or about 5 ng/ml to about 25 ng/ml.
85. The method of embodiment 84, wherein the concentration of the FGF2 in the gonadal induction medium is about 5 ng/mL or about 10 ng/mL.
86. The method of any one of embodiments 71-85, wherein the RAPM in the gonadal induction medium is an RAR agonist, optionally wherein the RAPM comprises retinoic acid (RA) and/or TTNPB.
87. The method of embodiment 86, wherein:
   (a) the RAPM is RA, wherein the concentration of RA in the gonadal induction medium is about 0.5 μM to about 2 μM; and/or
   (b) the RAPM is TTNPB, wherein the concentration of TTNPB in the gonadal induction medium is about 0.2 μM to about 1 μM.
88. The method of any one of embodiments 72-87, wherein within the gonadal induction medium:
(A) the apoptosis inhibitor is Y-27632, optionally wherein the concentration of Y-27632 is about 5 μM to about 20 μM; or
(B) the apoptosis inhibitor comprises Chroman1, Emricasan, and Trans-ISRIB, optionally wherein the concentration of Chroman1 is about 30 nM to about 70 nM, the concentration of Emricasan is about 2 μM to about 10 μM, and the concentration of Trans-ISRIB is about 0.2 μM to about 2 μM.
89. The method of embodiment 88, wherein within the gonadal induction medium:
(A) the concentration of Y-27632 is about 10 μM: or
(B) the concentration of Chroman1 is about 50 nM, the concentration of Emricasan is about 5 μM, and the concentration of Trans-ISRIB is about 0.7 μM.
90. The method of any one of embodiments 1-89, wherein the period of time for culturing in gonadal induction medium is about 5 days to about 21 days.
91. The method of embodiment 90, wherein the period of time for culturing in gonadal induction medium is about 7 days to about 14 days.
92. The method of any one of embodiments 1-91, wherein the gonadal cell population comprises ovarian somatic cells.
93. The method of any one of embodiments 1-92, wherein the gonadal cell population consists of ovarian somatic cells.
94. The method of any one of embodiments 1-93, wherein at least 20% of cells within the gonadal cell population are FOXL2-positive cells.
95. The method of any one of embodiments 1-94, wherein at least 20% of the cells within the gonadal cell population are NR2F2-positive cells.
96. The method of any one of embodiments 1-95, wherein at least 20% of the cells within the gonadal cell population are KRT19-positive cells
97. The method of any one of embodiments 1-96, wherein at least 90% of the gonadal cell population are FOXL2-positive cells. NR2F2-positive cells, and/or KRT-19 positive cells.
98. The method of embodiment 97, wherein the gonadal cell population consists essentially of FOXL2-positive cells, NR2F2-positive cells, and/or KRT-19 positive cells.
99. The method of embodiment 97 or 98, wherein the FOXL2-positive cells comprise granulosa cells; wherein the NR2F2-positive cells comprise stroma cells and/or granulosa cells; and wherein the KRT-19 positive cells comprise ovarian epithelial cells.
100. The method of any one of embodiments 1-99, wherein:
   (I) the amount of NR2F2-expressing cells in the gonadal cell population is increased; and/or
   (II) the amount of FOXL2-expressing cells in the gonadal cell population is increased; and/or
   (III) the amount of RUNX1-expressing cells in the gonadal cell population is increased; and/or
   (IV) the amount of WNT6-expressing cells in the gonadal cell population is increased; and/or
   (V) the amount of NR5A1-expressing cells in the gonadal cell population is increased; and/or
   (VI) the amount of OSR1-expressing cells in the gonadal cell population is increased; and/or
   (VII) the amount of LHX9-expressing cells in the gonadal cell population is increased; and/or
   (VIII) the amount of EMX2-expressing cells in the gonadal cell population is increased,
as compared to a corresponding gonadal cell population generated by a method wherein the intermediate mesoderm induction medium does not comprise RAPM.
101. The method of any one of embodiments 1-99, wherein:
   (I) the amount of NR2F2-expressing cells in the gonadal cell population is increased; and/or
   (II) the amount of FOXL2-expressing cells in the gonadal cell population is increased; and/or
   (III) the amount of RUNX1-expressing cells in the gonadal cell population is increased; and/or
   (IV) the amount of WNT6-expressing cells in the gonadal cell population is increased; and/or
   (V) the amount of NR5A1-expressing cells in the gonadal cell population is increased; and/or
   (VI) the amount of OSR1-expressing cells in the gonadal cell population is increased,
as compared to a corresponding gonadal cell population generated by a method wherein the intermediate mesoderm induction medium comprises a lower concentration of RAPM.
102. The method of any one of embodiments 1-99, wherein:
   (I) the amount of NR2F2-expressing cells in the gonadal cell population is increased; and/or
   (II) the amount of FOXL2-expressing cells in the gonadal cell population is increased; and/or
   (III) the amount of RUNX1-expressing cells in the gonadal cell population is increased; and/or
   (IV) the amount of WNT6-expressing cells in the gonadal cell population is increased; and/or
   (V) the amount of NR5A1-expressing cells in the gonadal cell population is increased; and/or
   (VI) the amount of OSR1-expressing cells in the gonadal cell population is increased; and/or
   (VII) the amount of LHX9-expressing cells in the gonadal cell population is increased; and/or
   (VIII) the amount of EMX2-expressing cells in the gonadal cell population is increased,
as compared to a corresponding gonadal cell population generated by a method wherein the intermediate mesoderm induction medium does not comprise glycogen synthase kinase-3 inhibitor.
103. The method of any one of embodiments 1-99, wherein:
   (I) the amount of NR2F2-expressing cells in the gonadal cell population is increased; and/or
   (II) the amount of FOXL2-expressing cells in the gonadal cell population is increased; and/or
   (III) the amount of RUNX1-expressing cells in the gonadal cell population is increased; and/or
   (IV) the amount of WNT6-expressing cells in the gonadal cell population is increased; and/or
   (V) the amount of NR5A1-expressing cells in the gonadal cell population is increased; and/or
   (VI) the amount of OSR1-expressing cells in the gonadal cell population is increased; and/or
   (VII) the amount of LHX9-expressing cells in the gonadal cell population is increased; and/or
   (VIII) the amount of EMX2-expressing cells in the gonadal cell population is increased,
as compared to a corresponding gonadal cell population generated by a method wherein the intermediate mesoderm induction medium comprises a lower concentration of glycogen synthase kinase-3 inhibitor.
104. The method of any one of embodiments 1-99, wherein:
   (I) the amount of LHX1 expression in the second intermediate cell population is increased; and/or
   (II) the amount of PAX2 expression in the second intermediate cell population is increased; and/or
   (III) the amount of WT1 expression in the second intermediate population is increased; and/or
   (IV) the amount of RUNX1 expression in the second intermediate cell population is increased; and/or
   (V) the viability of the second intermediate cell population is increased; and/or
   (VI) the cell morphology of the second intermediate cell population is more uniform,
as compared to a corresponding second intermediate cell population generated by a method wherein the intermediate mesoderm induction medium does not comprise RAPM.
105. The method of any one of embodiments 1-99, wherein:
   (I) the amount of LHX1 expression in the second intermediate cell population is increased; and/or
   (II) the amount of PAX2 expression in the second intermediate cell population is increased; and/or
   (III) the amount of WT1 expression in the second intermediate population is increased; and/or
   (IV) the amount of RUNX1 expression in the second intermediate cell population is increased; and/or
   (V) the viability of the second intermediate cell population is increased; and/or
   (VI) the cell morphology of the second intermediate cell population is more uniform,
as compared to a corresponding second intermediate cell population generated by a method wherein the intermediate mesoderm induction medium comprises a lower concentration of RAPM
106. The method of any one of embodiments 1-99, wherein:
   (I) the amount of LHX1 expression in the second intermediate cell population is increased; and/or
   (II) the amount of PAX2 expression in the second intermediate cell population is increased; and/or
   (III) the amount of WT1 expression in the second intermediate population is increased; and/or
   (IV) the amount of RUNX1 expression in the second intermediate cell population is increased; and/or
   (V) the viability of the second intermediate cell population is increased; and/or
   (VI) the cell morphology of the second intermediate cell population is more uniform,
as compared to a corresponding second intermediate cell population generated by a method wherein the intermediate mesoderm induction medium does not comprise glycogen synthase kinase-3 inhibitor.
107. The method of any one of embodiments 1-99, wherein:
   (I) the amount of LHX1 expression in the second intermediate cell population is increased; and/or
   (II) the amount of PAX2 expression in the second intermediate cell population is increased; and/or
   (III) the amount of WT1 expression in the second intermediate population is increased; and/or
   (IV) the amount of RUNX1 expression in the second intermediate cell population is increased; and/or
   (V) the viability of the second intermediate cell population is increased; and/or
   (VI) the cell morphology of the second intermediate cell population is more uniform,
as compared to a corresponding second intermediate cell population generated by a method wherein the intermediate mesoderm induction medium comprises a lower concentration of glycogen synthase kinase-3 inhibitor.
108. The method of any one of embodiments 1-99, wherein:
   (I) the potential of the second intermediate cell population to differentiate into NR2F2-expressing gonadal cells is increased; and/or
   (II) the potential of the second intermediate cell population to differentiate into FOXL2-expressing gonadal cells is increased; and/or
   (III) the potential of the second intermediate cell population to differentiate into RUNX1-expressing gonadal cells is increased; and/or
   (IV) the potential of the second intermediate cell population to differentiate into WNT6-expressing gonadal cells is increased; and/or
   (V) the potential of the second intermediate cell population to differentiate into NR5A1-expressing gonadal cells is increased; and/or
   (VI) the potential of the second intermediate cell population to differentiate into OSR1-expressing gonadal cells is increased,
as compared to a corresponding second intermediate cell population generated by a method wherein the intermediate mesoderm induction medium does not comprise RAPM.
109. The method of any one of embodiments 1-99, wherein:
   (I) the potential of the second intermediate cell population to differentiate into NR2F2-expressing gonadal cells is increased; and/or
   (II) the potential of the second intermediate cell population to differentiate into FOXL2-expressing gonadal cells is increased; and/or
   (III) the potential of the second intermediate cell population to differentiate into RUNX1-expressing gonadal cells is increased; and/or
   (IV) the potential of the second intermediate cell population to differentiate into WNT6-expressing gonadal cells is increased; and/or
   (V) the potential of the second intermediate cell population to differentiate into NR5A1-expressing gonadal cells is increased; and/or
   (VI) the potential of the second intermediate cell population to differentiate into OSR1-expressing gonadal cells is increased,
as compared to a corresponding second intermediate cell population generated by a method wherein the intermediate mesoderm induction medium comprises a lower concentration of RAPM
110. The method of any one of embodiments 1-99, wherein:
   (I) the potential of the second intermediate cell population to differentiate into NR2F2-expressing gonadal cells is increased; and/or
   (II) the potential of the second intermediate cell population to differentiate into FOXL2-expressing gonadal cells is increased; and/or
   (III) the potential of the second intermediate cell population to differentiate into RUNX1-expressing gonadal cells is increased; and/or
   (IV) the potential of the second intermediate cell population to differentiate into WNT6-expressing gonadal cells is increased; and/or
   (V) the potential of the second intermediate cell population to differentiate into NR5A1-expressing gonadal cells is increased; and/or
   (VI) the potential of the second intermediate cell population to differentiate into OSR1-expressing gonadal cells is increased,
as compared to a corresponding second intermediate cell population generated by a method wherein the intermediate mesoderm induction medium does not comprise glycogen synthase kinase-3 inhibitor.
111. The method of any one of embodiments 1-99, wherein:
   (I) the potential of the second intermediate cell population to differentiate into NR2F2-expressing gonadal cells is increased; and/or
   (II) the potential of the second intermediate cell population to differentiate into FOXL2-expressing gonadal cells is increased; and/or
   (III) the potential of the second intermediate cell population to differentiate into RUNX1-expressing gonadal cells is increased; and/or
   (IV) the potential of the second intermediate cell population to differentiate into WNT6-expressing gonadal cells is increased; and/or
   (V) the potential of the second intermediate cell population to differentiate into NR5A1-expressing gonadal cells is increased; and/or
   (VI) the potential of the second intermediate cell population to differentiate into OSR1-expressing gonadal cells is increased,
as compared to a corresponding second intermediate cell population generated by a method wherein the intermediate mesoderm induction medium comprises a lower concentration of glycogen synthase kinase-3 inhibitor.
112. The method of any one of embodiments 1-111, wherein:
   (I) the amount of FOXL2 expression in the gonadal cell population is decreased; and/or
   (II) the amount of expression of NR1H4 and/or KITLG in the gonadal cell population is decreased; and/or
   (III) the amount of KRT-19 expression in the gonadal cell population is increased; and/or
   (IV) the amount of cytoplasmic KRT-19 expression in the gonadal cell population is increased; and/or
   (V) the amount of expression of MSLN, LRRN4, and/or TMEM151A in the gonadal cell population is increased,
as compared to a corresponding gonadal cell population generated by a method wherein the gonadal induction medium comprises a lower concentration of RAPM.
113. The method of any one of embodiments 1-111, wherein:
   (I) the amount of FOXL2 expression in the gonadal cell population is decreased; and/or
   (II) the amount of expression of NR1H4 and/or KITLG in the gonadal cell population is decreased; and/or
   (III) the amount of KRT-19 expression in the gonadal cell population is increased; and/or
   (IV) the amount of cytoplasmic KRT-19 expression in the gonadal cell population is increased; and/or
   (V) the amount of expression of MSLN, LRRN4, and/or TMEM151A in the gonadal cell population is increased,
as compared to a corresponding gonadal cell population generated by a method wherein the gonadal induction medium does not comprise a RAPM.
114. The method of any one of embodiments 1-111, wherein:
   (I) the amount of FOXL2 expression in the gonadal cell population is decreased; and/or
   (II) the amount of expression of NR1H4 and/or KITLG in the gonadal cell population is decreased; and/or
   (III) the amount of KRT-19 expression in the gonadal cell population is increased; and/or
   (IV) the amount of cytoplasmic KRT-19 expression in the gonadal cell population is increased; and/or
   (V) the amount of expression of MSLN, LRRN4, and/or TMEM151A in the gonadal cell population is increased,
as compared to a corresponding gonadal cell population generated by a gonadal induction step comprising contacting with an RAPM for a shorter period of time.
115. The method of any one of embodiments 1-114, wherein the pluripotent stem cells are mammalian stem cells.
116. The method of any one of embodiments 1-115, wherein the pluripotent stem cells are human pluripotent stem cells.
117. The method of any one of embodiments 1-115, wherein the pluripotent stem cells are bovine stem cells.
118. The method of any one of embodiments 1-115, wherein the pluripotent stem cells are murine pluripotent stem cells.
119. The method of any one of embodiments 115-118, wherein the pluripotent stem cells are embryonic stem cells or induced pluripotent stem cells.
120. The method of any one of embodiments 1-6 and 10-119, wherein the gonadal population comprises one or more populations selected from granulosa cells, ovarian stroma cells and epithelial cells or a combination thereof.
121. The method of any one of embodiments 1-6 and 10-120, wherein the gonadal population comprises a mixture of granulosa cells, ovarian stroma cells and epithelial cells
122. A gonadal cell population produced by the method of any one of embodiments 1-6 and 10-121.
123. The gonadal cell population of embodiment 122, wherein the gonadal population comprises one or more populations selected from granulosa cells, ovarian stroma cells and epithelial cells or a combination thereof.
124. The gonadal cell population of embodiment 122 or embodiment 123, wherein the gonadal population comprises a mixture of granulosa cells, ovarian stroma cells and epithelial cells
125. A first intermediate cell population produced by the method of any one of embodiments 7 and 13-119.
126. A second intermediate cell population produced by the method of any one of embodiments 6-11 and 13-119.
127. A method of producing a granulosa cell, the method comprising:
culturing a pluripotent stem cell in the presence of activin A, a glycogen synthase kinase-3 inhibitor, and a ROCK inhibitor to produce an incipient mesoderm-like cell (iMeLC),
culturing the iMeLC in the presence of FGF2, a glycogen synthase kinase-3 inhibitor, and a ROCK inhibitor for a first period of time,
reducing an amount of the ROCK inhibitor in the iMeLC culture and then culturing the iMeLC for a second period of time to produce an intermediate mesoderm cell, and
culturing the intermediate mesoderm cell in the presence of follistatin, BMP4, FGF2, and a ROCK inhibitor to produce the granulosa cell.
128. The method of embodiment 127, wherein the glycogen synthase kinase-3 inhibitor is CHIR99021.
129. The method of embodiment 127 or 128, wherein the ROCK inhibitor is Y-27632 or CET.
130. The method of any one of embodiments 127-129, wherein the pluripotent stem cell is cultured for about 56 to 72 hours.
131. The method of any one of embodiments 127-130, wherein the pluripotent stem cell is cultured for about 65 hours.
132. The method of any one of embodiments 127-131, wherein pluripotent stem cell is cultured in a medium comprising the activin A, glycogen synthase kinase-3 inhibitor, and ROCK inhibitor, and wherein the medium is replaced with fresh medium every about 24 hours.
133. The method of any one of embodiments 127-132, wherein the first period of time is about 24 hours.
134. The method of any one of embodiments 127-133, wherein the second period of time is about 5 or 6 days.
135. The method of any one of embodiments 127-134, wherein the iMeLC is cultured in a medium comprising the FGF2, Glycogen synthase kinase-3 inhibitor, and ROCK inhibitor for the first period of time, and wherein after the first period of time a portion of the medium is replaced with a medium comprising FGF2 and glycogen synthase kinase-3 inhibitor but not a ROCK inhibitor.
136. The method of embodiment 135, wherein the portion of the medium is about 80% of the medium.
137. The method of embodiment 135 or 136, wherein the method further comprises replacing a second portion of the medium every about 48 hours during the second time period.
138. The method of embodiment 137, wherein the second portion of the medium is about 80% of the medium.
139. The method of any one of embodiments 127-138, wherein the intermediate mesoderm cell is cultured for a period of about 5-7 days.
140. The method of any one of embodiments 127-139, wherein the intermediate mesoderm cell is cultured in a medium comprising the follistatin, BMP4, FGF2, and ROCK inhibitor, and wherein a portion of the medium is replaced with fresh medium every about 24 to 48 hours.
141. The method of any one of embodiments 127-140, wherein the iMeLC expresses Brachyury.
142. The method of any one of embodiments 127-139, wherein the intermediate mesoderm cell expresses OSR1, PAX2, and LHX1.
143. The method of any one of embodiments 127-142, wherein the granulosa cell expresses FOXL2 and CONNEXIN43.
144. The method of any one of embodiments 127-143, wherein the pluripotent stem cell is a human induced pluripotent stem cell.
145. A population comprising granulosa cells produced by the method of any one of embodiments 127-144.
146. The method or population of any one of embodiments 1-145, wherein:
   (a) one or more of the culturing steps comprises adherent culture; and/or
   (b) one or more of the culturing steps comprises 3-dimensional organoid culture.
147. The method or population of any one of embodiments 1-146, wherein one or more of the culturing steps comprises adherent culture.
148. The method or population of any one of embodiments 1-147, wherein one or more of the culturing step comprises 3-dimensional organoid culture.
149. An in vitro stem cell-derived gonadal somatic cell population comprising FOXL2-expressing cells, NR2F2-expressing cells and/or KRT-19 expressing cells, optionally wherein the gonadal somatic cell population is an ovarian somatic cell population.
150. The gonadal somatic cell population of embodiment 149, wherein the population comprises at least a first cell type expressing FOXL2, a second cell type expressing NR2F2, and a third cell type expressing KRT-19.
151. The gonadal somatic cell population of embodiment 149 or 150, wherein:
(a) at least 20% of cells within the cell population are FOXL2-positive cells; and/or
(b) at least 20% of the cells within the cell population are NR2F2-positive cells; and/or
(c) at least 20% of the cells within the gonadal cell population are KRT19-positive cells.
152. The gonadal somatic cell population of any one of embodiments 149-151, wherein at least 90% of the gonadal cell population are FOXL2-positive cells, NR2F2-positive cells, and/or KRT-19 positive cells; optionally wherein:
the gonadal somatic cell population consists essentially of FOXL2-positive cells, NR2F2-positive cells, and/or KRT-19 positive cells.
153. The gonadal somatic cell population of embodiment 151 or 152, wherein the FOXL2-positive cells comprise granulosa cells; wherein the NR2F2-positive cells comprise ovarian stromal cells and/or granulosa cells; and wherein the KRT-19 positive cells comprise ovarian epithelial cells.
154. The gonadal somatic cell population of any one of embodiments 149-153, wherein the cell population is derived in a process comprising:
(a) culturing pluripotent stem cells in a mesoderm induction medium comprising Activin A for a first period of time to produce a first intermediate cell population;
(b) culturing the first intermediate cell population in an intermediate mesoderm induction medium comprising an retinoic acid pathway modulator (RAPM) for a second period of time, thereby producing a second intermediate cell population; and
(c) culturing the second intermediate cell population in a gonadal induction medium comprising BMP and FGF, and optionally follistatin, for a third period of time to produce the gonadal cell population.

### EXAMPLES

The application may be better understood by reference to the following non-limiting examples, which are provided as exemplary embodiments of the application. The following examples are presented in order to more fully illustrate embodiments and should in no way be construed, however, as limiting the broad scope of the application. While certain embodiments of the present application have been shown and described herein, it will be obvious that such embodiments are provided by way of example only. Numerous variations, changes, and substitutions may occur to those skilled in the art without departing from the spirit and scope of the invention. It should be understood that various alternatives to the embodiments described herein may be employed in practicing the methods described herein.

### Example 1: Media Formulation

In some embodiments, at least about any one of: 30%, 40%, 50%, 60%, 70%, 80%, 81%, 82%, The following media were prepared for use in one or more examples provided below.

TABLE 1
GK2 Medium Formulation

| GK2 medium | | 50 | mL | 20 | mL | 10 | mL | 1 | mL | Final | |
|---|---|---|---|---|---|---|---|---|---|---|---|
| GMEM | Fridge | 40.335 | mL | 16.134 | mL | 8.07 | mL | 0.81 | mL | | |
| KSR | −20° C. | 7.5 | mL | 3 | mL | 1.5 | mL | 150 | μL | 15% | |
| 2-ME | Fridge | 50 | μL | 20 | μL | 10 | μL | 1 | μL | 0.1 | mM |
| Glutamax | RT-shelf | 500 | μL | 200 | μL | 100 | μL | 10 | μL | 2 | mM L-glut |
| Pen/Strep | −20/fridge | 500 | μL | 200 | μL | 100 | μL | 10 | μL | 1X | |
| NEAA | Fridge | 500 | μL | 200 | μL | 100 | μL | 10 | μL | 0.1 | mM |
| Pyruvate | Fridge | 500 | μL | 200 | μL | 100 | μL | 10 | μL | 1 | mM |

TABLE 2
IM Medium Formulation

| IM medium | | | | | | | | | | Final | |
|---|---|---|---|---|---|---|---|---|---|---|---|
| GK2 medium | Use within | 50 | mL | 20 | mL | 10 | mL | 1 | mL | | |
| | 2 weeks of | | | | | | | | | | |
| | reconstituting | | | | | | | | | | |
| Thermo-stable FGF2 | −20° C. | 100 | μL | 40 | μL | 20 | μL | 2 | μL | 20 | ng/ml |
| (10 μg/mL) | | | | | | | | | | | |
| CHIR (10 mM) | −20° C. | 5 | μL | 2 | μL | 1 | μL | 0.1 | μL | 1 | μM |
| Prepare fresh by adding | | | | | | | | | | | |
| inducers to GK2 media | | | | | | | | | | | |
| Rock inhibitor | −20° C. | 50 | μL | 20 | μL | 10 | μL | 1 | μL | 10 | μM |

TABLE 3
Granulosa Basic Medium Formulation

| Granulosa Basic Medium | | | | | | | | | | Final | |
|---|---|---|---|---|---|---|---|---|---|---|---|
| GK2 medium | Use within 2 | 50 | mL | 20 | mL | 10 | mL | 1 | mL | | |
| | weeks of | | | | | | | | | | |
| | reconstituting | | | | | | | | | | |
| Follistatin (20 μg/mL) | −20° C. | 50 | μL | 20 | μL | 10 | μL | 1 | μL | 25 | ng/ml |
| BMP4 (50 μg/mL) | −20° C. | 10 | μL | 4 | μL | 2 | μL | 0.2 | μL | 10 | ng/ml |
| Thermo-stable FGF2 | −20° C. | 25 | μL | 10 | μL | 5 | μL | 0.5 | μL | 5 | ng/ml |
| (10 μg/mL) | | | | | | | | | | | |
| Prepare fresh by adding | | | | | | | | | | | |
| inducers to GK2 media | | | | | | | | | | | |
| Rock inhibitor | −20° C. | 50 | μL | 20 | μL | 10 | μL | 1 | μL | 10 | μM |

TABLE 4
Granulosa Double Medium Formulation

| Granulosa Double Medium | | | | | | | | | | Final | |
|---|---|---|---|---|---|---|---|---|---|---|---|
| GK2 medium | Use within 2 | 50 | mL | 20 | mL | 10 | mL | 1 | mL | | |
| | weeks of | | | | | | | | | | |
| | reconstituting | | | | | | | | | | |
| Follistatin (20 μg/mL) | −20° C. | 100 | μL | 40 | μL | 20 | μL | 2 | μL | 50 | ng/mL |
| BMP4 (50 μg/mL) | −20° C. | 20 | μL | 8 | μL | 4 | μL | 0 | μL | 20 | ng/ml |
| Thermo-stable FGF2 | −20° C. | 50 | μL | 20 | μL | 10 | μL | 1 | μL | 10 | ng/ml |
| (10 μg/mL) | | | | | | | | | | | |
| Prepare fresh by adding | | | | | | | | | | | |
| inducers to GK2 media | | | | | | | | | | | |
| Rock inhibitor | −20° C. | 50 | μL | 20 | μL | 10 | μL | 1 | μL | 10 | μM |

TABLE 5
Granulosa BMP7 Medium Formulation

| Granulosa BMP7 Medium | | | | | | | | | | Final | |
|---|---|---|---|---|---|---|---|---|---|---|---|
| GK2 medium | Use within 2 | 50 | mL | 20 | mL | 10 | mL | 1 | mL | | |
| | weeks of | | | | | | | | | | |
| | reconstituting | | | | | | | | | | |
| Follistatin (20 μg/mL) | −20° C. | 50 | μL | 20 | μL | 10 | μL | 1 | μL | 25 | ng/ml |
| BMP7 (50 μg/mL) | −20° C. | 10 | μL | 4 | μL | 2 | μL | 0.2 | μL | 10 | ng/mL |
| Thermo-stable FGF2 | −20° C. | 25 | μL | 10 | μL | 5 | μL | 0.5 | μL | 5 | ng/ml |
| (10 μg/mL) | | | | | | | | | | | |
| Prepare fresh by adding | | | | | | | | | | | |
| inducers to GK2 media | | | | | | | | | | | |
| Rock inhibitor | −20° C. | 50 | μL | 20 | μL | 10 | μL | 1 | μL | 10 | μM |

TABLE 6
Alternative 50 ng Differentiation Formulation

| 50 ng Differentiation | | | | | | | | | | Final | |
|---|---|---|---|---|---|---|---|---|---|---|---|
| Medium | | | | | | | | | | | |
| GK2 medium | Use within 2 | 50 | mL | 20 | mL | 10 | mL | 1 | mL | | |
| | weeks of | | | | | | | | | | |
| | reconstituting | | | | | | | | | | |
| FGF9 (100 μg/mL) | −20° C. | 25 | μL | 10 | μL | 5 | μL | 0.5 | μL | 50 | ng/ml |
| CHIR (10 mM) | −20° C. | 5 | μL | 2 | μL | 1 | μL | 0.1 | μL | 1 | μM |
| Prepare fresh by adding | | | | | | | | | | | |
| inducers to GK2 media | | | | | | | | | | | |
| Rock inhibitor | −20° C. | 50 | μL | 20 | μL | 10 | μL | 1 | μL | 10 | μM |

TABLE 7
CHIR Differentiation Medium Formulation

| CHIR Differentation | | | | | | | | | | Final | |
|---|---|---|---|---|---|---|---|---|---|---|---|
| Medium | | | | | | | | | | | |
| GK2 medium | Use within 2 weeks | 50 | mL | 20 | mL | 10 | mL | 1 | mL | | |
| | of reconstituting | | | | | | | | | | |
| Dorsomorphin (2 mM) | −20° C. | 50 | μL | 20 | μL | 10 | μL | 1 | μL | 2 | μM |
| CHIR (10 mM) | −20° C. | 15 | μL | 6 | μL | 3 | μL | 0.3 | μL | 3 | μM |
| Prepare fresh by adding | | | | | | | | | | | |
| inducers to GK2 media | | | | | | | | | | | |
| Rock inhibitor | −20° C. | 50 | μL | 20 | μL | 10 | μL | 1 | μL | 10 | μM |

TABLE 8
MEF Medium Formulation

| | | | | | | | | | | |
|---|---|---|---|---|---|---|---|---|---|---|
| MEF Medium | | 50 | mL | 20 | mL | 10 | mL | 1 | mL | Final |
| DMEM | Fridge | 44.5 | mL | 17.8 | mL | 8.9 | mL | 0.890 | mL | |
| FBS | Fridge | 5 | mL | 2 | mL | 1 | mL | 100 | μL | 10% |
| Pen/Strep | Fridge | 500 | μL | 200 | μL | 100 | μL | 10 | μL | 1X |
| Primocin | −20° C. | 50 | μL | 20 | μL | 10 | μL | 1 | μL | 1X |
| Store @ 4 C for 1 month | | | | | | | | | | |

TABLE 9
iMeLC Medium Formulation

| IM medium | | | | | | | | | | Final | |
|---|---|---|---|---|---|---|---|---|---|---|---|
| GK2 medium | Use within | 50 | mL | 20 | mL | 10 | mL | 1 | mL | | |
| | 2 weeks of | | | | | | | | | | |
| | reconstituting | | | | | | | | | | |
| Activin A (50 μg/mL) | −20° C. | 50 | μL | 20 | μL | 10 | μL | 1 | μL | 50 | ng/mL |
| CHIR (10 mM) | −20° C. | 15 | μL | 6 | μL | 3 | μL | 0.3 | μL | 3 | μM |
| Rock inhibitor | −20° C. | 50 | μL | 20 | μL | 10 | μL | 1 | μL | 10 | μM |
| Prepare fresh by adding | | | | | | | | | | | |
| inducers to GK2 media | | | | | | | | | | | |

### Example 2: Production of iMeLC Cells

Differentiation of stem cells progresses through various stages that can be identified by changes in gene expression. Described is a method to generate ovarian somatic cells by progressively converting stem cells through a series of steady stable states that mimic embryonic development of the fetal ovary. For each step, conditions were optimized for maximum purity and efficiency in order to obtain pure homogeneous ovarian somatic cell cultures. Two key intermediate steps were identified in granulosa cell differentiation as well as marker genes for each of those steps. The following protocol was tested with two different stem cell lines. For robust differentiation, the expression of certain markers was optimized at each step by changing the concentration and the duration of inducers (e.g., cytokines or small molecules). Such optimization is built into the protocol for a robust, universal method that would work with cell lines of different genetic backgrounds.

Before working with the cells, each well of a 12-well plate was coated with 610 μL fibronectin solution, which was prepared with 600 μL PBS (at room temperature)+10 μL Fibronectin (1 mg/ml; Millipore, FC010, which was kept on ice), and incubated at 37° C. for 1 hour. Afterwards, media from human induced Pluripotent Stem Cells (hiPSCs) was removed and the cells were washed gently with PBS (room temp). PBS was then removed and 500 μL of 1:1 TrypLE Select+500 μL of 0.5 mM EDTA solution (37° C.) was added to each well of iPSCs, followed by 2-3 min incubation at 37° C. The cells were then removed from the wells by squirting 1 mL MEF medium into the wells and then resuspended into a single cell suspension by pipetting them 3-5 times. The cells were counted and then centrifuged at 1200 rpm for 5 mins. The centrifuged cells were resuspended in GK2 media to obtain about 10×10⁶ cells/mL. Cells were counted again and 70K cells X number of wells to be induced were transferred to a 1.5 mL tube (or 15 mL if many wells are collected). These hiPSCs were centrifuged and resuspended into the iMeLC medium to obtain about 70k cells/mL.

Fibronectin coating solution was removed from the coated wells, and the wells were washed once with PBS. 1 mL of iMeLC medium containing the 70k iPSCs was added to the wells. The plated cells were cultured in an incubator for 56-72 hours (preferably 65 hours) at 37° C. with 5% CO₂. The iMeLC media was changed every 24 hours. About 2-4 million iMeLCs per well were produced from one 12 well plate.

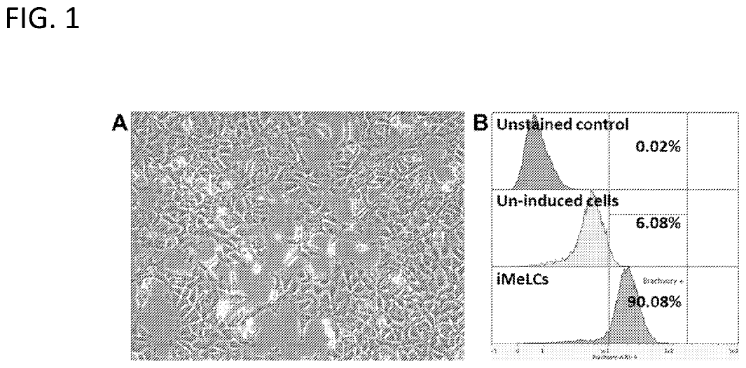

iMeLCs were characterized by expression of transcription factors such as—Brachyury. Fluorescently labelled antibodies were used to detect the protein expression in iMeLCs. FIG. 1A shows the morphology of iMeLCs under phase contrast imaging and FIG. 1B shows the high expression of Brachyury (bottom panel) at 48 hrs after induction of the stem cells. Cells not treated with CHIR and activin (middle panel) show low to no expression of Brachyury.

### Example 3: Production of IM Cells

Cultures of iMeLCs were extended to 65 hours to ensure a homogenous population of iMeLCs that were then induced to IM cells. IM cells are characterized by the expression of PAX2, OSR1 and LHX1. Culture conditions were identified to extend the cultures of IM cells through several passages and multiple weeks. This enables the expansion of IM cells and also provides a pure, homogenous culture that can be differentiated to downstream lineages. Furthermore, the bulk of IM cells can be frozen to ensure superior batch control for reproducible differentiation of granulosa cells in the next steps.

iMeLC media in the wells from Example 2 was removed and washed with 1 mL PBS. 500 μL of TrypLE was added to each well, and the plate was incubated at 37° C. for 2 min. 500 μL MEF medium was added to each well and pipetted 3 times up and down to achieve a single cell suspension. The suspension was transferred to 1.5 mL tube (or 15 mL if many wells are collected), and the remainder of cells was collected with another 500 μL of MEF medium.

The cell suspension was centrifuged at 1200 RPM for 5 min and the supernatant was discarded. The centrifuged cells were resuspended in 1 ml of GK2 medium and the cells were counted. About 720,000 cells (from 24 well plate: 30.000 cells per well) were added to 12 mL IM medium (as prepared in Example 1, but without the ROCK inhibitor). 12 μL ROCK inhibitor Y-27632 (1000×) or 36 μL of CET (3:1000) (final concentration of CET in media: 50 nM Chroman1, 5 μM Emricasan, 0.7 μM Trans-ISRIB) was added to obtain 60,000 cells/mL. 500 μL of the solution was placed in each well of fibronectin coated 24-well plate (about 30,000 cells/well). The plates were cultured for 24 hours, and then 80% of the media was replaced with fresh IM media without the ROCK inhibitor. 80% of the media was again replaced every other day until the cells became confluent (about 5-6 days). Once the cells were confluent, the cells were passaged to fresh fibronectin-coated 24-well plates, with at least 50,000 cells per well after passage (25k/cm²). About 4-5 million IM cells/24 well plate were produced.

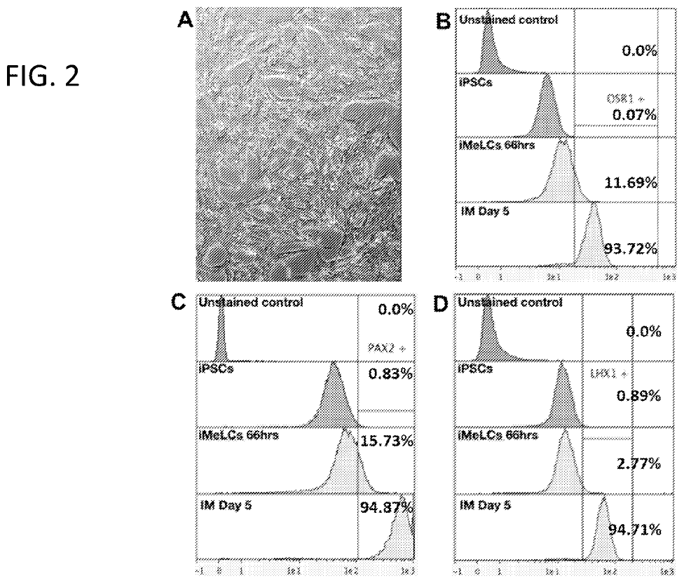

FIG. 2 shows the morphology of day 5 IM cells in culture under phase contrast imaging. The cells were stained for OSR1, PAX2 and LHX1; IM cells (bottom panels in each of FIGS. 2B-D) show high expression of all tested markers as compared to iPSCs and iMeLCs. Unimodal distribution of the signal signifies the purity and homogeneity of the IM cells in culture.

### Example 4: Granulosa and Differentiation into Other Lineages

Depleted media from IM cells was removed and the cells were washed with 500 μL PBS at room temperature. 200 μL TrypLE (at 37° C.) was added and the cells were incubated at 37° C. for 2 min. 300 μL MEF medium (room temp) was added to each well and pipetted 3 times up and down to achieve a single cell suspension. The suspension was transferred to a 15 mL tube. The remainder of the cells were collected with another 500 μL of MEF medium. The transferred cells were centrifuged at 1200 RPM for 5 mins and the supernatant was discarded. The cells were resuspended in 1 ml of GK2 medium and the cells were counted.

500 μL of differentiation media (about 30,000 cells) with ROCK inhibitor (either Y-27 or CET—as previously described) were added to each well of a fibronectin coated 24 well plate. Separate experiments were conducted with each of the following differentiation media (at 37° C.): granulosa basic, granulosa double, granulosa BMP7, alternative differentiation medium (50 ng FGF9), CHIR differentiation medium, and IM (control).

In each case, the plates were cultured for 24 hours, and then 80% of the differentiation media was replaced with fresh media. 80% of the media was again replaced every other day until the cells became confluent (about 5-6 days). Once the cells were confluent, the process was repeated to passage IM cells to fresh fibronectin coated 24 well plates, with at least 30.000 cells per well after each passage (15k cells/cm²). After differentiation of granulosa cells for about 5-7 days, 2-4 million granulosa cells per 24 well plate were produced.

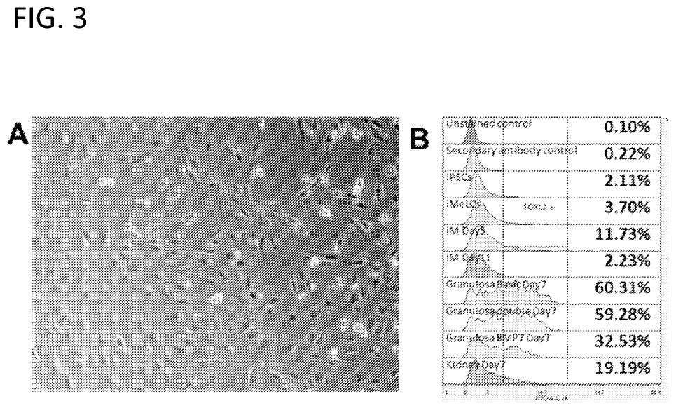

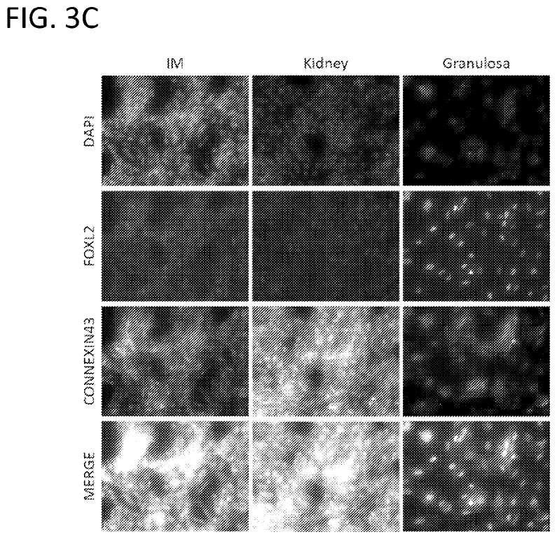

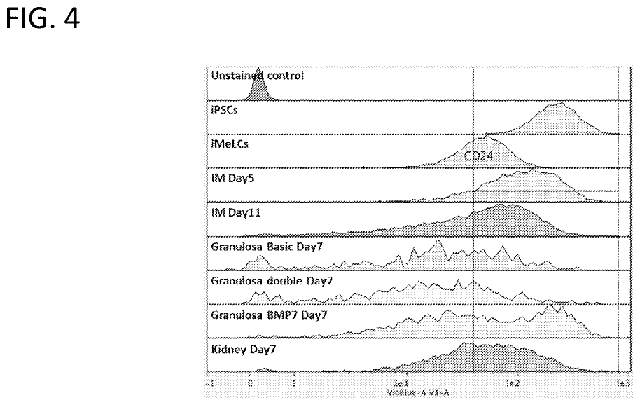

On day 7 of differentiation, cells were collected for staining with FOXL2, a transcription factor that marks the granulosa cells. FIG. 3A shows the morphology of granulosa cells (left). FIG. 3B shows distribution of FOXL2+ cells in different culture conditions, with the highest percentage of FOXL2+ cells in granulosa differentiation. Immunofluorescence staining for FOXL2 and CONNEXIN43 shows expression of FOXL2 in granulosa cells as compared to IM and kidney cells (FIG. 3C).

### Example 5: Identification of Intermediate Stages of Granulosa Differentiation

IM cells are multi-potent progenitor cells that can differentiate to multiple lineages including ovarian somatic cells. There are multiple types of ovarian somatic cells; progenitor cell(s) were identified that are committed to ovarian somatic lineage and can differentiate to these cell types. CD24 was identified as a highly expressed cell surface marker in the early differentiation, but its expression diminishes as cells commit to ovarian somatic fate. This provides us a guiding trajectory of granulosa cell differentiation for downstream applications.

### Example 6: Generation of Intermediate Mesoderm and Fetal Ovarian Somatic Cells, Including Fox12+ Granulosa Cells from Human Pluripotent Stem Cells

### Media Formulation

TABLE 10
IM-induction Medium Formulation

| Intermediate mesoderm | | | | | | | | | | Final | |
|---|---|---|---|---|---|---|---|---|---|---|---|
| induction medium | | | | | | | | | | | |
| GK2 medium | Use within | 50 | mL | 20 | mL | 10 | mL | 1 | mL | | |
| | 2 weeks of | | | | | | | | | | |
| | reconstituting | | | | | | | | | | |
| Thermo-stable FGF2 | −20° C. | 100 | μL | 40 | μL | 20 | μL | 2 | μL | 20 | ng/ml |
| (10 μg/mL) | | | | | | | | | | | |
| CHIR (10 mM) | −20° C. | 10 | μL | 4 | μL | 2 | μL | 0.2 | μL | 2 | μM |
| RA (100 μM) | −20° C. | 500 | μl | 200 | μl | 100 | μl | 10 | μl | 1 | μM |
| Prepare fresh by adding | | | | | | | | | | | |
| inducers to GK2 media | | | | | | | | | | | |
| Rock inhibitor | −20° C. | 50 | μL | 20 | μL | 10 | μL | 1 | μL | 10 | μM |

### A. Generation of a First Intermediate Cell Population Expressing Markers Representative of Mesoderm.

A 12-well plate was coated with 610 μL fibronectin solution, which was prepared with 600 μL PBS (at room temperature)+10 μL Fibronectin (1 mg/ml; Millipore, FC010, which was kept on ice), and incubated at 37° C. for 1 hour. Afterwards, media from human induced Pluripotent Stem Cells (hiPSCs) was removed and the cells were washed gently with PBS (room temp). PBS was then removed and 500 μL of 1:1 TrypLE Select was added to each well of iPSCs, followed by 2-3 min incubation at 37° C. The cells were then removed from the wells by squirting 1 mL MEF medium into the wells and then resuspended into a single cell suspension by pipetting them 3-5 times. The cells were counted and then centrifuged at 1200 rpm for 5 mins. The centrifuged cells were resuspended in GK2 media to obtain about 10×10⁶ cells/mL. Cells were counted again and 60K cells X number of wells to be plated were transferred to a 1.5 mL tube (or 15 mL if many wells are collected). These hiPSCs were centrifuged and resuspended into the iMeLC medium (see Table 9) (a mesoderm induction medium) to obtain about 60k cells/mL

Fibronectin coating solution was removed from the coated wells, and 1 mL of iMeLC medium containing the 60k iPSCs was added to the wells. The plated cells were cultured in an incubator for 56-72 hours (preferably 65 hours) at 37° C. with 5% CO2. The iMeLC media was changed every 24 hours. About 2-4 million iMeLCs per well were produced from one 12 well plate.

iMeLCs were characterized by expression of transcription factors such as—Brachyury. Fluorescently labelled antibodies were used to detect the protein expression in iMeLCs. FIG. 1A shows the morphology of iMeLCs under phase contrast imaging and FIG. 1B shows the high expression of Brachyury (bottom panel) at 48 hrs after induction of the stem cells. Cells not treated with CHIR and activin (middle panel) show low to no expression of Brachyury.

### B. Generation of a Second Intermediate Cell Population Expressing Markers Representative of Intermediate Mesoderm.

iMeLC media in the wells of the first intermediate cell population (expressing markers representative of mesoderm) was removed and washed with 1 mL PBS. 500 μL of TrypLE was added to each well, and the plate was incubated at 37° C. for 2 min. 500 μL MEF medium was added to each well and pipetted 3 times up and down to achieve a single cell suspension. The suspension was transferred to 1.5 mL tube (or 15 mL if many wells are collected), and the remainder of cells was collected with another 500 μL of MEF medium.

The cell suspension was centrifuged at 1200 RPM for 5 min and the supernatant was discarded. The centrifuged cells were resuspended in 1 ml of GK2 medium and the cells were counted. About 720,000 cells (from 24 well plate: 30,000 cells per well) were added to 12 mL IM-induction medium (as prepared in Table 10, but without the ROCK inhibitor). 12 μL ROCK inhibitor Y-27632 (1000×) or 36 μL of CET (3:1000) (final concentration of CET in media: 50 nM Chroman1, 5 μM Emricasan, 0.7 μM Trans-ISRIB) was added to obtain 60,000 cells/mL. 500 μL of the solution was placed in each well of fibronectin coated 24-well plate (about 30,000 cells/well). The plates were cultured for 24 hours, and then 80% of the media was replaced with fresh IM-induction media without the ROCK inhibitor. 80% of the media was again replaced every other day until the cells became confluent (about 5-6 days). Once the cells were confluent, the cells were passaged to fresh fibronectin-coated 24-well plates, with at least 50,000 cells per well after passage (25k/cm²). About 4-5 million second intermediate cells were produced per 24 well plate.

To further determine the effect of the GSK3 inhibitor and a retinoic acid pathway modulator (RAPM) on the efficacy of induction of second intermediate cell population, subsequent to the replating of the first intermediate cell population as described above, the cells were incubated with IM-induction media with increasing levels of RAPM and GSK inhibitors. Retinoic acid (RA) treated IM cells show better survival and induction of gonadal somatic cell differentiation (Data not shown)

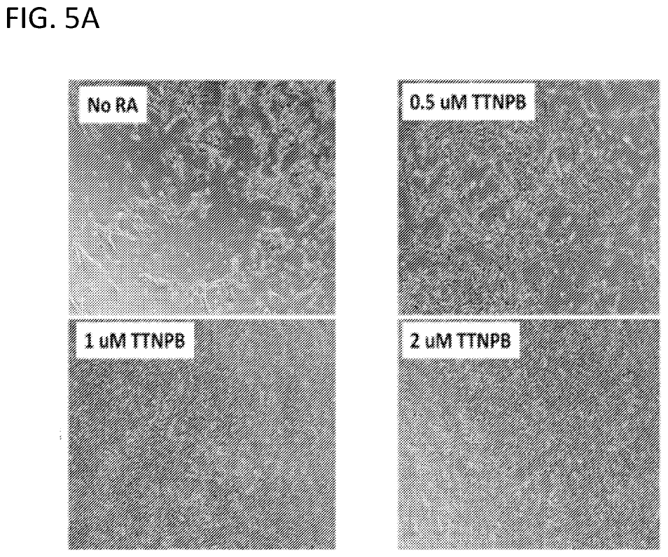

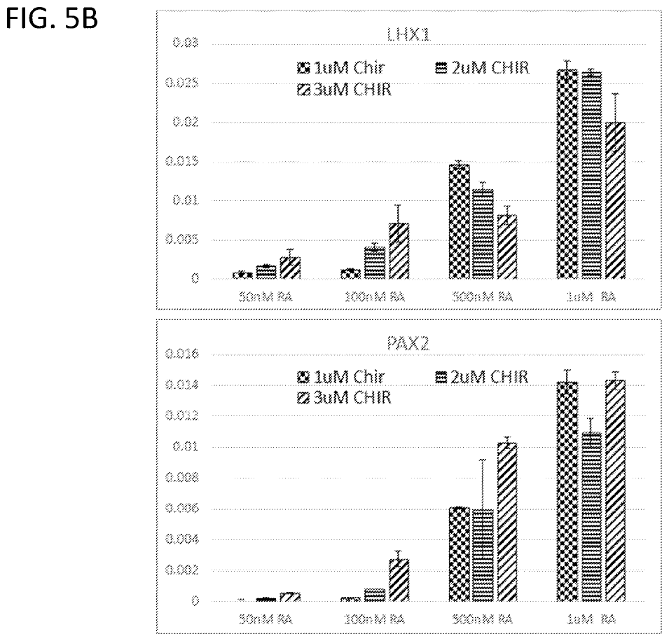

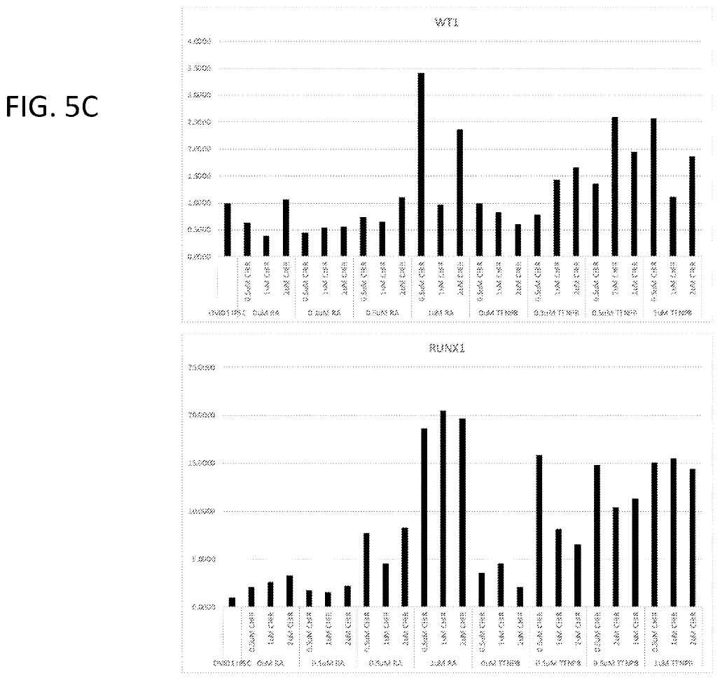

FIG. 5A shows the morphology of the resulting second intermediate cell population at 5 days under phase contrast imaging, after incubation of the first intermediate cell population in IM-induction media comprising GK2 medium, FGF2 and CHIR according to Table 10, and either 0.5 M, 1 μM or 2 μM of the RAPM TTNPB. As shown in FIG. 5A, an increase in the concentration of RA resulted in increased cell survival and uniform cellular morphology.

FIG. 5B shows the expression of intermediate mesoderm markers LHX1 and PAX2 in the resulting second intermediate cell population, after incubation of the first intermediate cell population in IM-induction media comprising GK2 medium and FGF2 at concentrations according to Table 10; either 1 μM, 2 μM or 3 μM of the GSK3 inhibitor CHIR; and either 50 nM, 100 nM, 500 nM or 1 μM of RA. As shown in FIG. 5B, an increase in the concentration of RA and/or CHIR resulted in increased expression of LHX1 (upper panel) and PAX2 (lower panel).

FIG. 5C shows the expression of intermediate mesoderm markers WT1 and RUNX1 in the resulting second intermediate cell population, after incubation of the first intermediate cell population in IM-induction media comprising GK2 medium and FGF2 at concentrations according to Table 10; either 0.5 μM, 1 μM or 2 μM of the GSK3 inhibitor CHIR; and either 0 μM, 0.1 μM, 0.5 μM, or 1 μM of RA or TTNPB. As shown in FIG. 5C, an increase in the concentration of RA and/or CHIR resulted in increased expression of WT1 (left panel) and RUNX1 (right panel).

### C. Generation of Gonadal Somatic Cells

The second intermediate cell population expressing markers representative of intermediate mesoderm were generated according to the protocols above, where the first intermediate cell population was incubated in IM-induction media comprising GK2 medium and FGF2 at concentrations according to Table 10; either 0.5 μM, 1 μM or 2 μM of the GSK3 inhibitor CHIR; and either 0 μM, 0.1 μM, 0.5 μM, or 1 μM of RA or TTNPB

Depleted media from the second intermediate cell population expressing markers representative of intermediate mesoderm was removed and the cells were washed with 500 μL PBS at room temperature. 200 μL TrypLE was added and the cells were incubated at 37° C. for 2 min. 300 μL MEF medium (room temp) was added to each well and pipetted 3 times up and down to achieve a single cell suspension. The suspension was transferred to a 15 mL tube. The remainder of the cells were collected with another 500 μL of MEF medium. The transferred cells were centrifuged at 1200 RPM for 5 mins and the supernatant was discarded. The cells were resuspended in 1 ml of GK2 medium and the cells were counted.

About 60.000 second intermediate population cells generated in each of the conditions described above were reconstituted in 500 μL of differentiation media with ROCK inhibitor (either Y-27632 or CET—as previously described), and replated to respective wells of a fibronectin coated 24 well plate. The respectively replated cells from the second intermediate population (generated under different concentrations of CHIR and RAPM) were incubated in the granulosa basic medium according to Table 3, with or without further supplement of 500 nM RA.

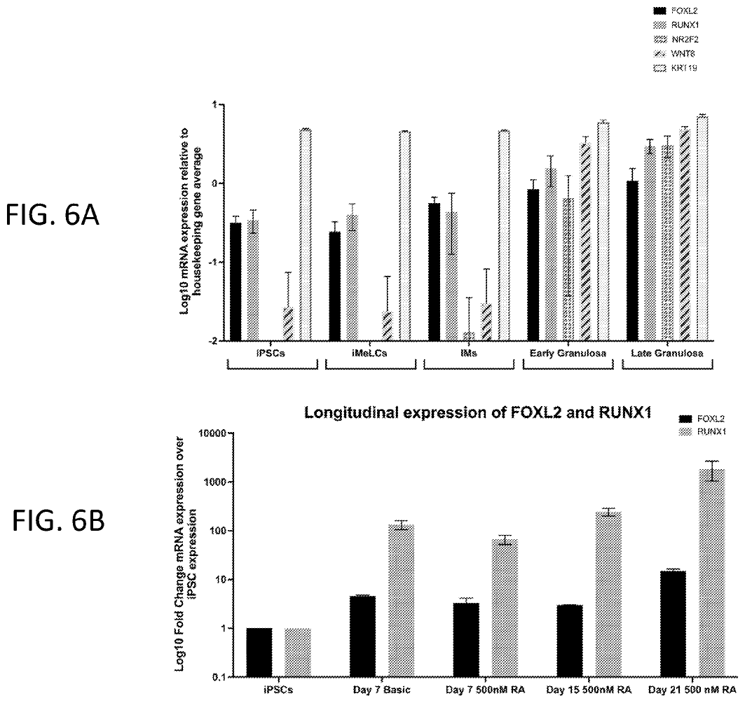

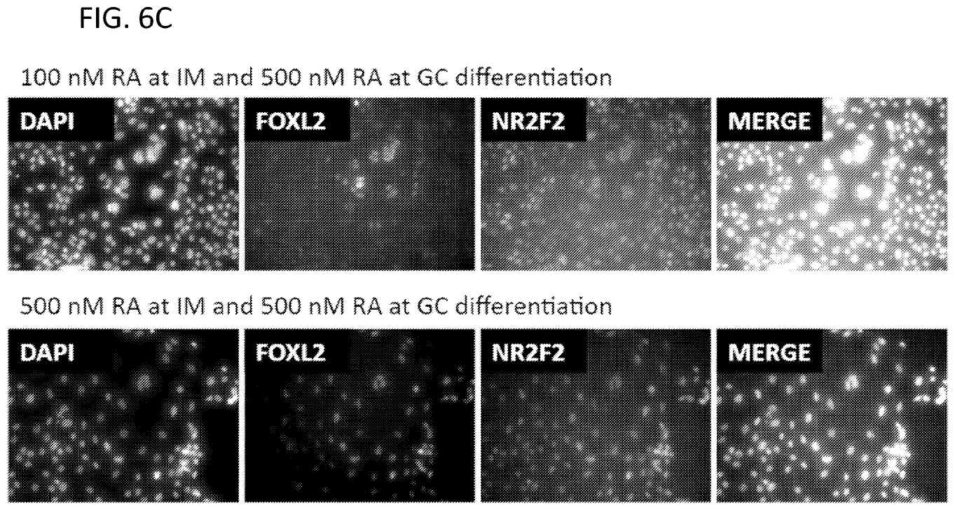

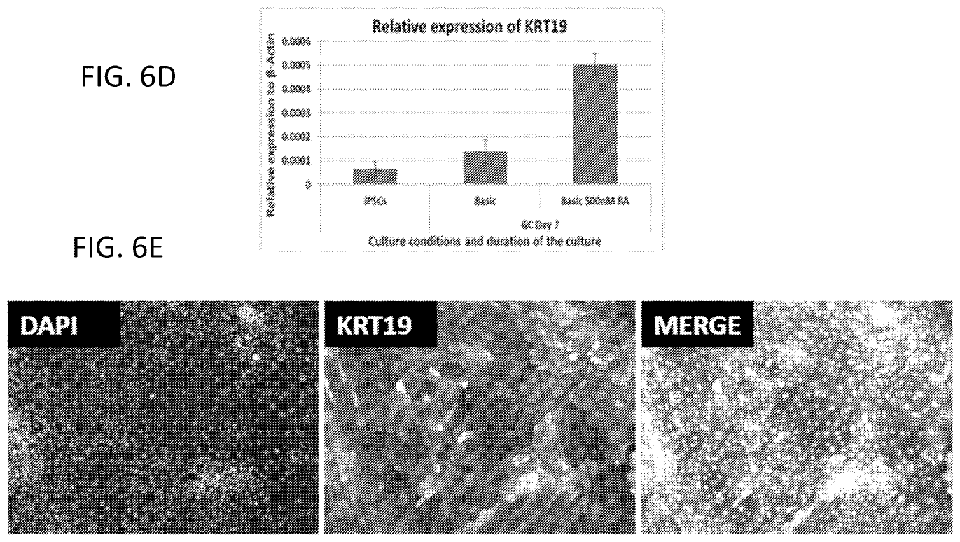

Cells were longitudinally collected for RNA-seq analysis, immunofluorescent staining, and flow cytometry. As assayed in a bulk RNAseq experiment, FIG. 6A indicates that in the absence of RA treatment at any stage, the resulting gonadal cells showed increased expression of gonadal somatic cell markers—FOXL2, RUNX1, NR2F2, WNT6 and KRT19 with FOXL2, a transcription factor that marks the granulosa cells. FIG. 6B indicates that in long term culture, incubation in differentiation medium comprising RA led to increased survival (cells without RA treatment did not survive by D21 of differentiation) and increased granulosa cell marker FOXL2 and RUNX1 expression.

As shown by the immunofluorescent staining in FIG. 6C, a higher concentration of RA in IM-induction media during the generation of the second intermediate cell population resulted in higher levels of FOXL2- and NR2F2-positive cells in the gonadal cell population. We simultaneously observed increased expression of KRT19 with RA treatment, as shown in FIG. 6D. This was also confirmed by immunostaining for KRT19 in continuous RA treated cells for 28 days. We observed a fine balance between length and concentration of RA treatment during generation of second intermediate cell populations and gonadal somatic cells leads to a variable % of FOXL2 and KRT19 positive cells (data not shown), which could be exploited to achieve a desirable heterogeneity for downstream applications.

### Example 7: Generation of Intermediate Mesoderm and Fetal Ovarian Somatic Cells, Including Fox12+ Granulosa Cells from Mouse Pluripotent Stem Cells

The granulosa differentiation protocol designed using human pluripotent stem cells was adapted for a mouse model system and resulted in induction of Fox12+ granulosa-like cells from mouse pluripotent stem cells. Mouse pluripotent stem cells were treated with growth factors on a 2-dimensional tissue culture environment to recapitulate early ovarian development. First, cells were directed towards a first intermediate cell population, then a second intermediate cell population, and then a fetal ovarian somatic cell fate. At each stage, cells were analyzed by qRT-PCR and protein staining to evaluate gene and protein expression of stage specific markers.

### A. Generation of a First Murine Intermediate Cell Population In Vitro

Mouse pluripotent stem cells were cultured, maintained and dissociated using previously published techniques (Chow et al. npg Regenerative Medicine 2020, PMID: 32351711). Prior to beginning the induction towards the first intermediate cell population induction, a 24 well plate was coated with 0.5 mL 100 μg/mL Matrigel diluted in DMEM-F12 for 1 hour at room temperature or overnight at 4° C. 50,000 mouse pluripotent stem cells were then seeded into each well of a 24-well plate in priming medium. Cells were grown in an incubator at 5% CO2, 37° C. for 48 hours. Cells were then treated with murine mesoderm induction medium (Table 14) for 48 hours. The resulting cells expressed high levels of the mesoderm marker brachyury by qRT-PCR and immunostaining (data not shown).

### B. Generation of a Second Murine Intermediate Cell Population In Vitro

The first intermediate cell population was washed 1× with room temperature sterile PBS, then treated with murine intermediate mesoderm induction medium (Table 15) for 48-72 hours. Resulting cells expressed intermediate mesoderm markers Pax2 and Lhx1 by qRT-PCR (data not shown).

### C. Generation of Mouse Fetal Ovarian Somatic Cells In Vitro

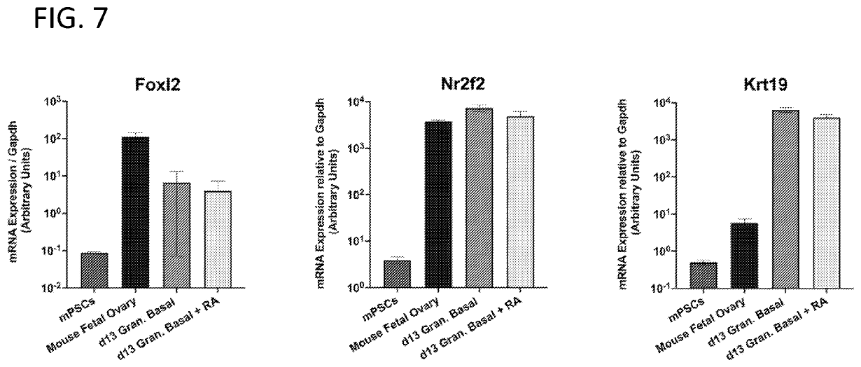

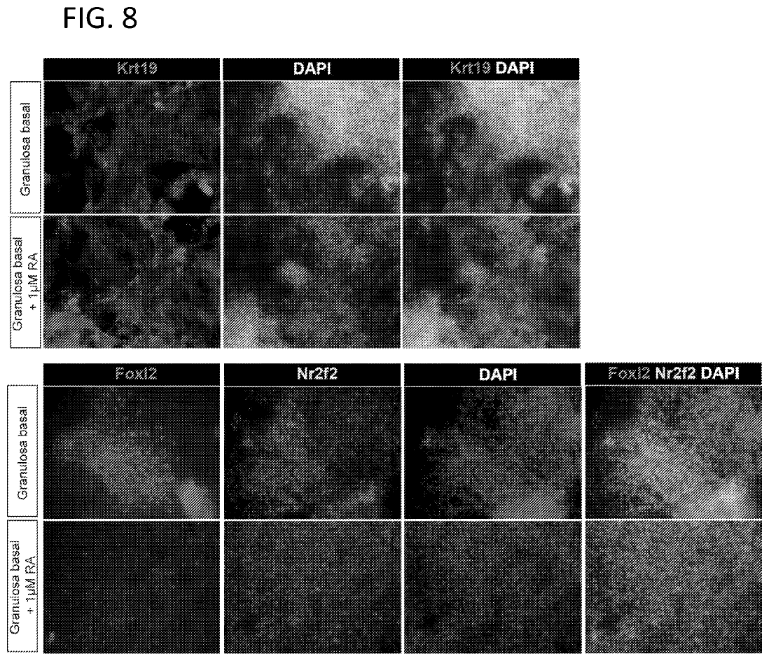

The second murine intermediate cell population was washed 1× with room temperature sterile PBS, then treated with murine gonadal induction medium (basal medium or basal medium supplemented with 1 μM RA; Table 16) for 7 days. The resulting cells were harvested for RT-PCR analysis and immunofluorescent staining. Immunofluorescent staining for Fox12 on cells treated with or without RA revealed that addition of 1 μM retinoic led to an increase in the total number of cells expressing Fox12 protein (FIG. 8, leftmost panel). As assayed by qRT-PCR, FIG. 7 shows that the resulting induced gonadal cells expressed comparable or higher levels of markers of granulosa cells (Fox12), stroma cells (Nr2f2) and ovarian epithelial cells (KRT19) as compared to fetal ovary cells. As shown by immunostaining in FIG. 8, the resulting induced gonadal cells expressed the protein markers for granulosa cells (Fox12), stroma cells (Nr2f2) and ovarian epithelial cells (Krt19)

### Media Formulations:

TABLE 11

| N2B27 basal medium | Total volume: 200 | ml |
|---|---|---|
| DMEM/F-12 (without glutamine) | 95 | ml |
| Neurobasal medium | 95 | ml |
| B27supplement | 4 | ml |
| N2 supplement | 2 | ml |
| Pen/Strep | 2 | ml |
| Glutamax | 2 | ml |
| B-mercaptoethanol (55 mM) | 0.2 | ml |

TABLE 12

| GK15 basal medium | Total volume: 25 | ml |
|---|---|---|
| GMEM | 20.225 | ml |
| KSR | 3.75 | ml |
| B-mercaptoethanol (55 mM) | 0.025 | ml |
| Glutamax | 0.25 | ml |
| Pen/Strep | 0.25 | ml |
| Non-essential amino acids | 0.25 | ml |
| (NEAA) | | |
| Sodium pyruvate | 0.25 | ml |

TABLE 13
Murine Priming Medium

N2B27 basal medium
12 ng/ml Fgf2
20 ng/ml activin A
1% multi-species knockout serum

TABLE 14
Murine Mesoderm Induction Medium

N2B27
4% KSR
30 ng/ml Bmp4
10 ng/ml activin A
12 ng/ml Fgf2

TABLE 15

| Murine intermediate mesoderm induction medium | 12000 μL | |
|---|---|---|
| DMEM/F12 | 11380.68 | μL |
| 4% multi-species knockout serum | 480 | μL |
| 50 U/ml penicillin, 50 μg/ml streptomycin | 120 | μL |
| 10 μM Y27632 | | |
| 100 nM retinoic acid | | |
| 30 ng/ml activin A | | |

TABLE 16

| Murine Gonadal induction medium | 25000 μL |
|---|---|
| GK15 basal medium | 25000 μL |
| Follistatin (25 μg/mL) | |
| BMP4 (50 μg/mL) | |
| Thermo stable FGF2 (10 ng/mL) | |
| Retinoic Acid (1 μM) | |

### Example 8: Induction of OSC with Different BMP Isoform

This example illustrates the effect of various BMP isoforms in the ability to induce gonadal somatic cells (such as the ovarian somatic cells—OSCs) from the second intermediate population. Gonadal somatic cells were induced in granulosa double medium comprising different BMP isoforms, and the expression of bipotential gonadal markers as well as that of granulosa markers were measured in the resulting cells.

A first intermediate cell population expressing markers representative of mesoderm, and a second intermediate cell population expressing markers representative of intermediate mesoderm were generated according to the protocols described in Example 6.

The replated cells from the second intermediate population were incubated in the granulosa double medium (also referred to as gonadal induction medium or OSC induction medium) according to Table 17, with 20 ng/mL of either BMP4, BMP2, BMP 7 or BMP15. The expression of bipotential gonadal markers (WT1, LHX9, GADD45G and GATA4) as well as granulosa markers (FOXL2, NR1H4 and KITLG) were measured by qPCR in the resulting cells.

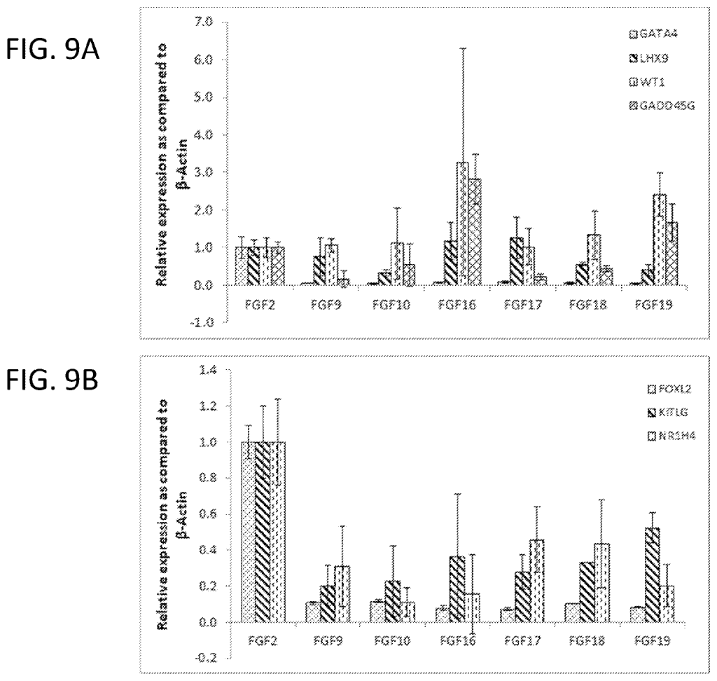

As shown in FIG. 9A, the expression of bipotential gonadal markers were either comparable or higher in OSC induction with medium comprising BMP2, BMP7 or BMP15 as compared to OSC induction with medium comprising BMP4.

As shown in FIG. 9B, the expression of granulosa markers were cither comparable or higher in OSC induction with a medium comprising BMP2, BMP7 or BMP15 as compared to OSC induction with a corresponding medium comprising BMP4.

These results indicate that OSCs could be induced using granulosa double medium comprising BMP4, BMP2, BMP7, BMP15, or a combination thereof.

TABLE 17
Granulosa Double Medium with different BMP isoforms

| Granulosa Double Medium | Final Concentration | |
|---|---|---|
| GK2 medium | | |
| Follistatin | 50 | ng/mL |
| BMP -- (BMP4, BMP2, BMP 7 or BMP 15) | 20 | ng/mL |
| Thermo-stable FGF2 | 10 | ng/mL |
| Prepare fresh by adding inducers to GK2 media | | |
| Rock inhibitor | 10 | μM |

### Example 9: Induction of OSC with Different Fibroblast Growth Factor (FGF) Family Members

Fibroblast growth factors (FGFs) are a large family of proteins that are important for signaling events in a wide variety of processes. This example illustrates the effect of various FGF family members in the ability to induce gonadal somatic cells (such as the ovarian somatic cells—OSCs) from the second intermediate population. Briefly, gonadal cells were induced in granulosa doublemedium comprising different FGFs, and the expression of bipotential gonadal markers as well as that of granulosa markers were measured in the resulting cells.

A first intermediate cell population expressing markers representative of mesoderm, and a second intermediate cell population expressing markers representative of intermediate mesoderm were generated according to the protocols described in Example 6.

The replated cells from the second intermediate population were incubated in the granulosa double medium (also referred to as gonadal induction medium or OSC induction medium) according to Table 18, with the same concentration of either FGF2, 9, 10, 16, 17, 18 or 19. The expression of bipotential gonadal markers (WT1, LHX9, GADD45G and GATA4) as well as granulosa markers (FOXL2, NR1H4 and KITLG) were measured in the resulting cells.

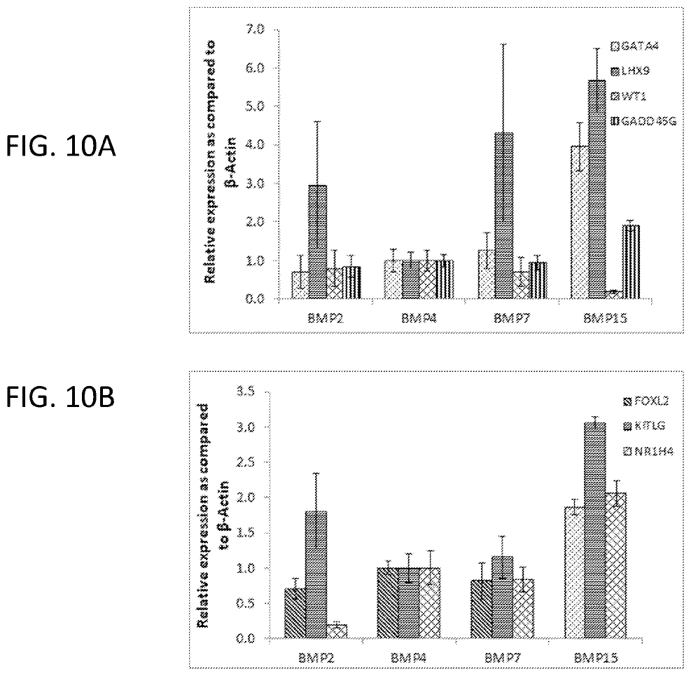

As shown in FIG. 10A, the expression of bipotential gonadal markers were either comparable or higher in OSC induction with a medium comprising FGF9, 10, 16, 17, 18 and 19 as compared to OSC induction with a corresponding medium comprising FGF2.

As shown in FIG. 9B, the expression of granulosa markers were comparable in OSC induction with a medium comprising FGF9, 10, 16, 17, 18 and 19 as compared to OSC induction with a corresponding medium comprising BMP4.

These results indicate that OSCs could be induced using granulosa double medium comprising FGF2, 9, 10, 16, 17, 18 or 19, or a combination thereof.

TABLE 18
Granulosa Double Medium with different BMP isoforms

| Granulosa double Medium | Final | |
|---|---|---|
| GK2 medium | | |
| Follistatin | 5 | ng/mL |
| BMP4 | 20 | ng/mL |
| FGF -- (FGF2, 9, 10, 16, 17, 18 or 19) | 10 | ng/mL |
| Prepare fresh by adding inducers to GK2 media | | |
| Rock inhibitor | 10 | μM |

### Example 10: OSCs Induced from Pluripotent Stem Cells are Functional

Granulosa cells become steroidogenic upon maturation—these cells are able to convert androgens such as testosterone into estrogens such as estradiol. One of the key enzymes responsible for steroidogenesis is Aromatase-CYP19A1.

To assay whether the gonadal somatic cells (such as the ovarian somatic cells—OSCs) generated from pluripotent stem cells are functional, expression of CYP19A1 was assayed by qPCR, and steroidogenesis was measured by ELISA for estradiol.

A first intermediate cell population expressing markers representative of mesoderm, and a second intermediate cell population expressing markers representative of intermediate mesoderm, and the OSCs were generated according to the protocols described in Example 6.

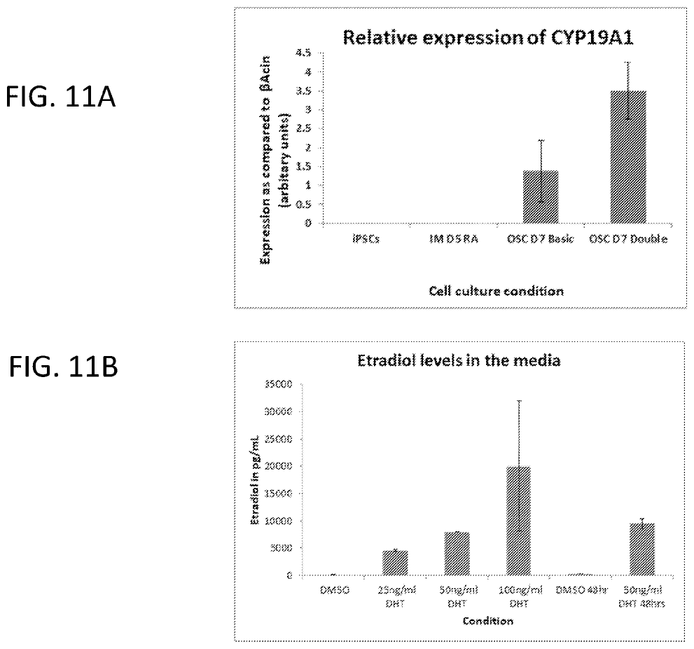

Expression of CYP19A1 was measured by qPCR in OSCs after 7 days of induction. As shown in FIG. 11A, the expression of CYP19A1 was increased in OSCs generated by basic induction media (basic) and further increased in OSCs generated by induction containing twice the growth factors as the basic induction media (double).

To further demonstrate this functionality of the granulosa cells, the estradiol secreted in the media after treatment of OSCs with dihydroxy-testosterone (dhT) was measured by ELISA. As shown in FIG. 11B, the observed estradiol levels in the media increased with higher concentration of dHT treatment after a 24-hr incubation. In addition, with a longer incubation (48 hrs), a further increase in estradiol levels was observed as compared to 24-hr incubation when OSCs were treated with 50 ng/ml dHT.

The results indicate that mature granulosa cells could be generated using the methods of OSC induction protocols in the examples described above.

### Example 11: Concentration Titration for BMP4, Follistatin and FGF for OSC Induction

In certain initial experiments, a medium comprising 10 ng/ml of BMP4, 25 ng/ml of Follistatin and 5 ng/ml of FGF2 was used for inductions of gonadal somatic cells (such as the ovarian somatic cells—OSCs) to test the differentiation potential of a second intermediate population. Various concentration titrations of BMP4, Follistatin and FGF were tested in OSC induction, and the resulting cells were examined for expression of bipotential gonadal markers as well as that of granulosa markers.

A first intermediate cell population expressing markers representative of mesoderm, and a second intermediate cell population expressing markers representative of intermediate mesoderm according to the protocols described in Example 6. The OSCs were induced based on the protocols described in Example 6, but with various concentrations of BMP4. Follistatin and FGF tested.

### BMP4

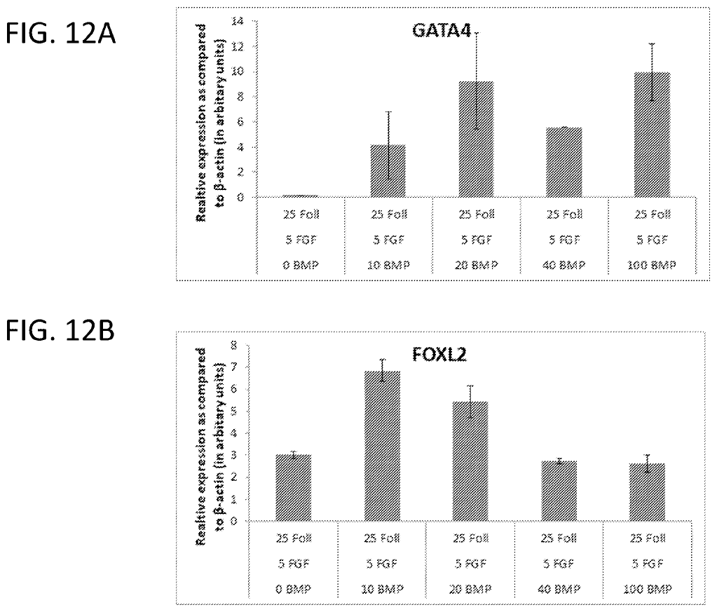

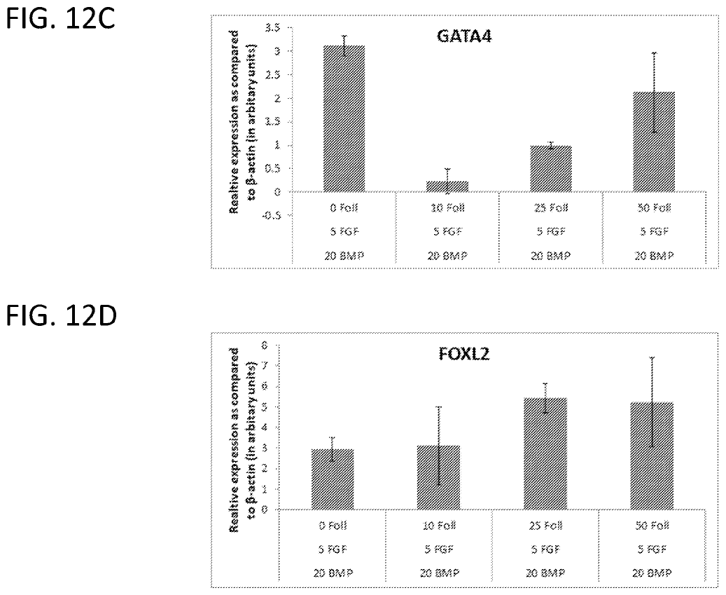

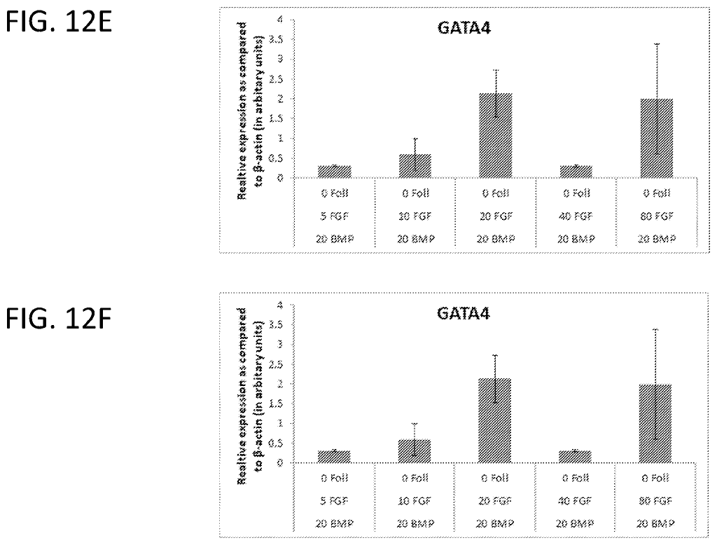

The OSC induction conditions were first tested by varying the BMP4 concentration. Inductions without any BMP4 led to poor survival, but all other tested concentrations of BMP4 led to similar comparable morphology. The gene expression of resulting cells was tested by qPCR for bi-potential gonadal cell markers (GATA4) as well as for granulosa cell markers (FOXL2) (FIGS. 12A and 12B respectively). Expression of GATA4 increased with higher BMP4 concentration, and FOXL2 expression appeared to peak at 10 ng/ml of BMP4 under the conditions tested.

For subsequent experiments, 20 ng/ml of BMP4 was used for OSC induction testing, based on the expression results for progenitor and mature OSC populations.

### Follistatin

Four different concentrations of follistatin were then tested in OSC induction, with the concentration of BMP4 kept constant at 20 ng/ml as previously described. The gene expression of resulting cells was tested by qPCR for bi-potential gonadal cell markers (GATA4) as well as for granulosa cell markers (FOXL2) (FIGS. 12C and 12D respectively). The GATA4 expression in FIG. 12C showed that it was possible to generate OSCs from progenitors in the absence of follistatin. The generation of mature OSCs were also examined by FOXL2 expression. As shown in FIG. 12D, a marginal increase in FOXL2 expression was observed at higher concentrations of follistatin. In subsequent experiments, OSC induction in the absence or at lower concentrations of follistatin was further explored.

### FGF2

Five different concentrations of FGF2 were then tested in OSC induction, with the concentration of BMP4 kept constant at 20 ng/mL and follistatin removed (Ong/mL). The gene expression of resulting cells were tested by qPCR for bi-potential gonadal cells (GATA4) as well as for granulosa cells (FOXL2) (FIGS. 12E and 12F respectively). As shown by FIGS. 12E and 12F, both GATA4 expression and FOXL2 expression reached saturation at 20 ng/ml of FGF2.

### Results

As shown by the comprehensive concentration titrations for BMP4, follistatin and FGF2 above, the results indicated that BMP4 promoted progenitor populations (bi-potential gonadal cells), but the effect appeared to saturate at 20 ng/ml for OSCs under the conditions tested. Similarly, we found the effect of FGF2 on the generation of progenitors as well as mature populations saturate at 20 ng/ml under the conditions tested. On the other hand, while removing follistatin altogether appeared to increase GATA4 expression, GATA4 expressing progenitors could potentially also give rise to other lineages besides OSCs. Therefore, the impact of follistatin concentration was also examined based on the results of FOXL2 expression. At 25 ng/ml of follistatin, a marginal increase in FOXL2 expression was observed but the FOXL2 expression did not increase with higher concentrations of follistatin. The results showed that OSC induction could be carried out without follistatin as well as with the various concentrations of follistatin as tested. Further experiments will be required to understand the relevance of follistatin in OSC induction.

### Example 12: Effect of RA Temporal and Concentration Gradient on Cell Fate to Pre-Granulosa Vs Epithelial Cells

From our previous experiments it had been shown that if RA was present during inductions of gonadal somatic cells (such as the ovarian somatic cells—OSCs) for more than 2 days, many more cells expressed higher levels of KRT19, as well as cytoplasmic KRT19, all of which were indicative of higher proportion of epithelial/mesothelial cells. On the other hand, if no RA was present during OSC induction, there would be a higher proportion of cells that expressed FOXL2, which was indicative of a higher proportion of granulosa cells.

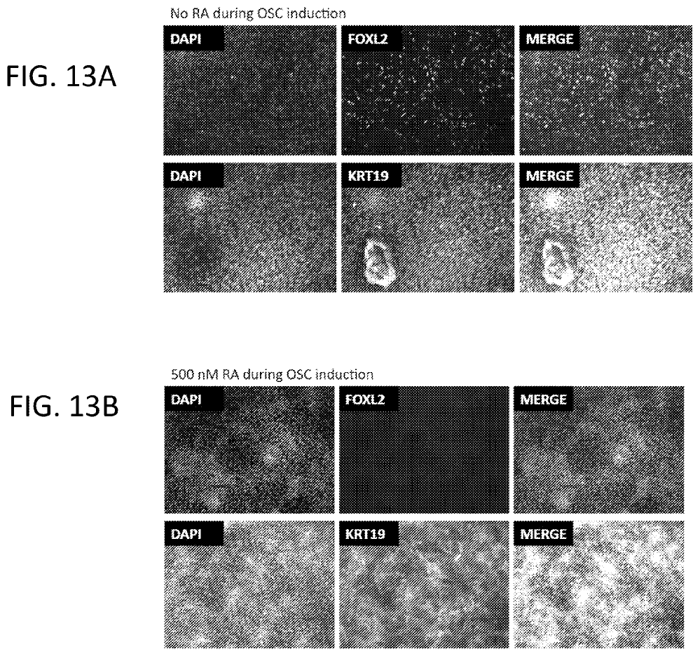

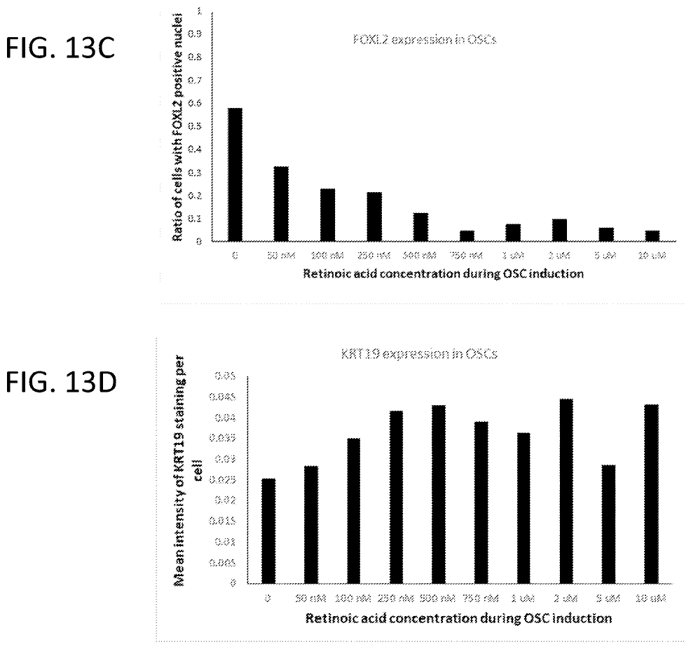

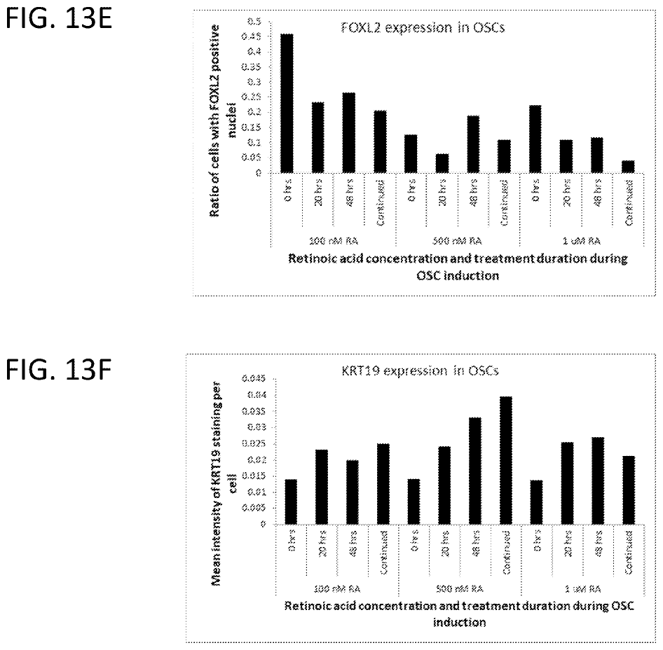

In order to further understand the effect of RA during OSC induction on the fine balance between epithelial/mesothelial cells versus granulosa cells, we created a 10-step concentration gradient of RA from 0 μM to 10 μM. With increasing concentration of RA we observed a decrease in percentage of nuclei positive for FOXL2 staining and a corresponding increase in KRT19 staining that peaked at around 500 nM concentration of RA (FIGS. 13A, 13B, 13C, 13D). Next, we tested if the length of exposure to RA would affect this balance between FOXL2- and KRT19-expressing cells, with the test conducted at three different concentrations of RA—100 nM. 500 nM, and 1 μM. For ¼th of the wells we did not add any RA, for the next ¼th of the wells we added RA for first 20 hrs, for another ¼th of the wells, we added RA for 48 hrs and for the remainder ¼th we continued the RA exposure for the length of the experiment (7 days). All the cells were fixed at D7 and stained for FOXL2 and KRT19. FOXL2 expression was quantified by the percent of FOXL2-positive nuclei; whereas KRT-19 expression was measured by the mean intensity of KRT-19 staining normalized to the number of nuclei based on DAPI staining.

Similar to the trend previously observed, even a short exposure to RA led to an increase in KRT19 positive cells and a decrease in FOXL2 positive cells (FIGS. 13E, 13F). The results confirmed that even a short pulse or a low concentration of RA is sufficient to induce and increase the epithelial/mesothelial cells during OSC induction.

### Example 13 Characterization of the OSCs Based on Gene Expression Profiles from: qPCR for Marker Genes; Single Cell RNAseq and Bulk RNAseq

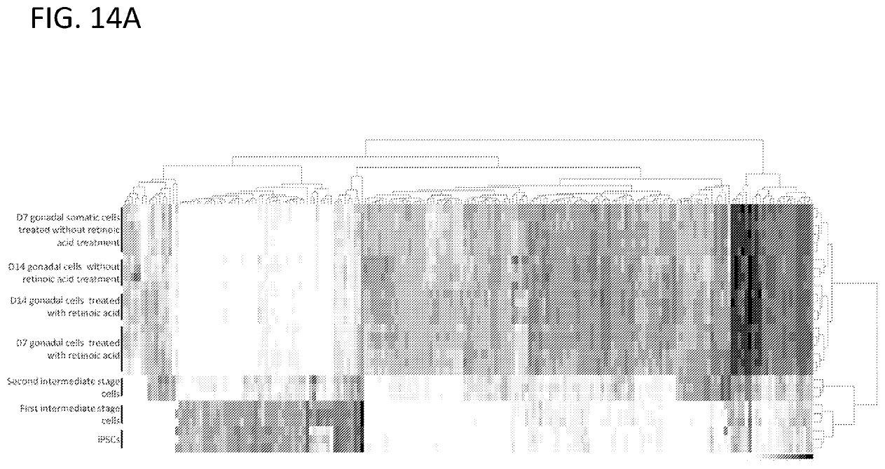

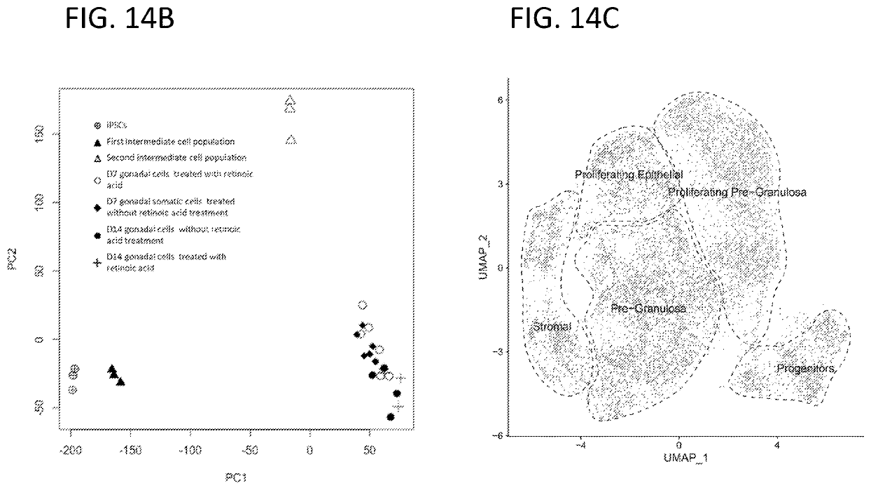

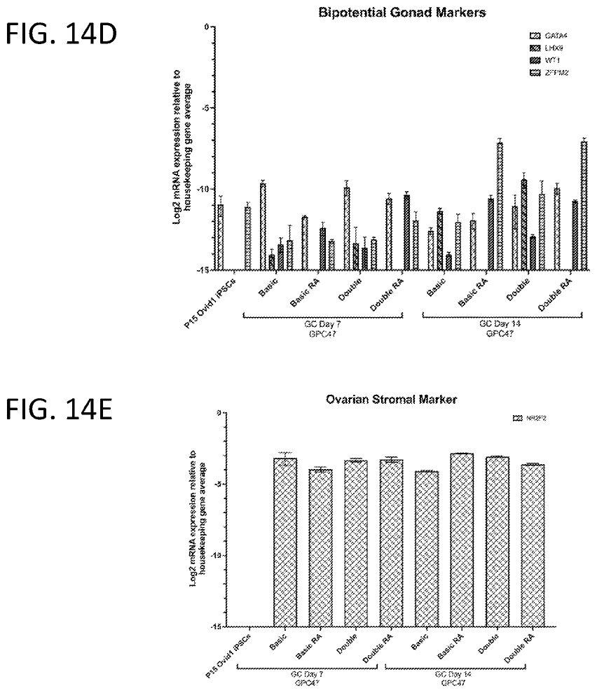

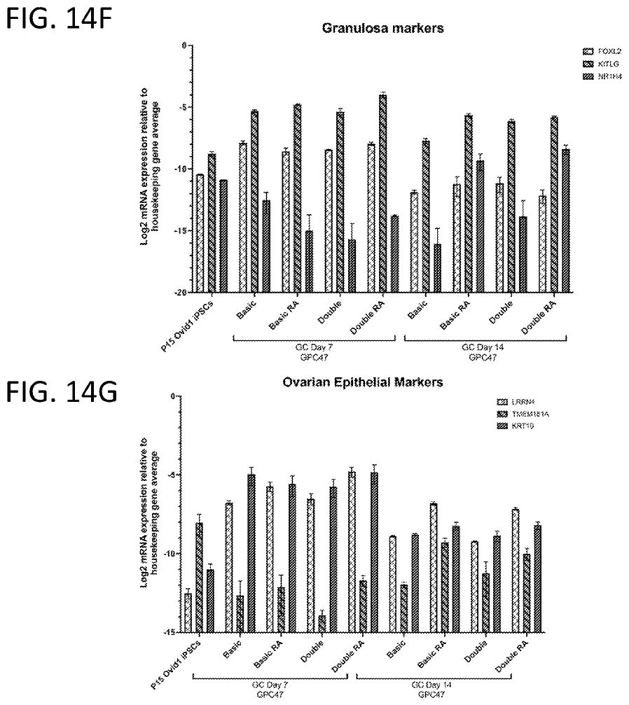

To characterize OSCs at gene expression level, we collected longitudinal samples for a bulk RNAseq experiment. Cells were snap-frozen as iPSCs, at first and second intermediate stages (i.e. first and second intermediate cell populations), and as OSCs (at D7 and D14 of OSC induction, with or without retinoic acid treatment). Heatmap for the top 200 highly variable genes and principal component analysis of the normalized and processed data indicated drastic changes in the gene expression profile during transition from the first intermediate stage to the second intermediate stage, and from the second intermediate stage to the OSCs (FIGS. 14A and 14B). We observed that the retinoic acid treated OSCs, exhibited increased expression in clusters of genes enriched for regulation of hormone levels and cell proliferation (FIG. 14A) To further characterize the heterogeneity of the differentiating OSCs, we collected Day 2 and 7 of OSCs with or without retinoic acid treatment and subjected them to single cell analysis using the 10× genomics platform according to the manufacturer's protocol. There were five distinct clusters of genes with increased expression, observed amongst more than 10.000 cells analyzed in this experiment. We observed that these clusters corresponded to pre-granulosa, dividing pre-granulosa, stromal cells, proliferative progenitors and epithelial cells as characterized by the marker gene expression (FIG. 14C). Furthermore, to validate these sequencing based gene expression readouts, we performed qPCR assays for biopotential gonadal markers-GATA4, WT1, LHX9 and ZFPM2; stromal cell marker-NR2F2; Granulosa cell markers-FOXL2, KITLG and NR1H4; and epithelial cell markers-KRT19, TMEM151A and LRRN4 (FIGS. 14D, 14E, 14F, 14G, respectively). For most of these genes we observed increased expression in day7 and day 14 after OSC induction under basic or double conditions with or without retinoic acid treatment.

### Example 14 qPCR and Immunofluorescence Assays on Induced Ovarian Somatic Cells

A first intermediate cell population expressing markers representative of mesoderm, and a second intermediate cell population expressing markers representative of intermediate mesoderm according to the protocols described in Example 6. The gonadal somatic cells (such as the ovarian somatic cells—OSCs) were induced based on the protocols described in Example 6.

To further verify the identity of OSCs, we analyzed the gene expression for granulosa marker genes-FOXL2 and bipotential gonadal genes-LHX9 and WT1. The expression of all these three genes was upregulated with longer culture duration. We also saw an increase in expression of markers for mature granulosa cells-KITLG and NR1H4 with longer culture duration (data not shown).

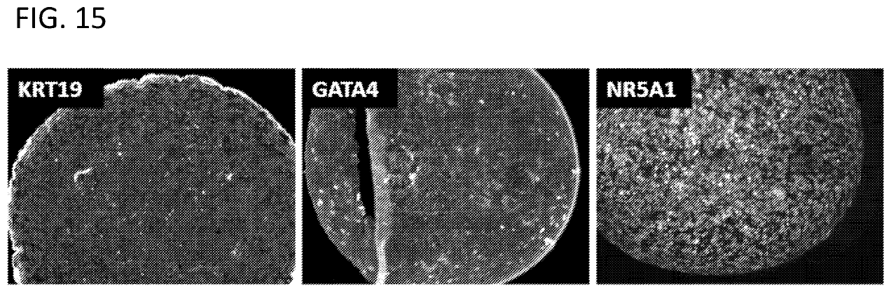

While qPCR assays are effective in assessing the gene expression in bulk, it cannot appropriately demonstrate the heterogeneity of the cell types present in the culture. To assess the heterogeneity as well as localization of different cell types in the OSC differentiation, cell aggregates were sectioned and stained for immunofluorescence imaging. As shown in FIG. 15, ovarian somatic cells were induced (as shown by GATA4 and NR5A1 expressing cells), as were ovarian epithelial/mesothelial cells (as shown by KRT-19 expressing cells).

The result showed that the differentiation protocol could be used to derive the described somatic cell types from pluripotent stem cells.

The present invention is not intended to be limited in scope to the particular disclosed embodiments, which are provided, for example, to illustrate various aspects of the invention. Various modifications to the compositions and methods described will become apparent from the description and teachings herein. Such variations may be practiced without departing from the true scope and spirit of the disclosure and are intended to fall within the scope of the present disclosure.

1. A method of producing a gonadal cell population, the method comprising:
(a) culturing pluripotent stem cells in a mesoderm induction medium comprising Activin A for a first period of time to produce a first intermediate cell population;
(b) culturing the first intermediate cell population in an intermediate mesoderm induction medium comprising an retinoic acid pathway modulator (RAPM) for a second period of time, thereby producing a second intermediate cell population; and
(c) culturing the second intermediate cell population in a gonadal induction medium comprising BMP and FGF, for a third period of time to produce the gonadal cell population.

2. A method of producing a gonadal cell population, the method comprising:
(a) culturing pluripotent stem cells in a mesoderm induction medium comprising Activin A for a first period of time to produce a first intermediate cell population;
(b) culturing the first intermediate cell population in an intermediate mesoderm induction medium comprising an retinoic acid pathway modulator (RAPM) for a second period of time, thereby producing a second intermediate cell population; and
(c) culturing the second intermediate cell population in a gonadal induction medium comprising follistatin, BMP4 and FGF, for a third period of time to produce the gonadal cell population.

3. A method of producing a gonadal cell population, the method comprising:
(a) culturing mesoderm or mesoderm-like cells in an intermediate mesoderm induction medium comprising a retinoic acid pathway modulator (RAPM) for a first period of time thereby producing a second intermediate cell population; and
(b) culturing the second intermediate cell population in a gonadal induction medium comprising BMP, and FGF for a second period of time to produce the gonadal cell population

4. A method of producing a gonadal cell population, the method comprising:
(a) culturing mesoderm or mesoderm-like cells in an intermediate mesoderm induction medium comprising a retinoic acid pathway modulator (RAPM) for a first period of time thereby producing a second intermediate cell population; and
(b) culturing the second intermediate cell population in a gonadal induction medium comprising follistatin, BMP4, and FGF for a second period of time to produce the gonadal cell population.

5. A method of producing a gonadal cell population, the method comprising:
culturing intermediate mesoderm or intermediate mesoderm-like cells in a gonadal induction medium comprising, BMP and FGF for a period of time to produce the gonadal cell population.

6. A method of producing a gonadal cell population, the method comprising:
culturing intermediate mesoderm or intermediate mesoderm-like cells in a gonadal induction medium comprising follistatin, BMP4 and FGF for a period of time to produce the gonadal cell population.

7. A method of producing a first intermediate cell population, the method comprising: culturing pluripotent stem cells in a mesoderm induction medium comprising Activin A and a glycogen synthase kinase-3 inhibitor for a period of time to produce a first intermediate cell population

8. A method of producing a second intermediate cell population, the method comprising:
culturing mesoderm or mesoderm-like cells in an intermediate mesoderm induction medium comprising a retinoic acid pathway modulator (RAPM) for a period of time, thereby producing the second intermediate cell population.

9. A method of producing a second intermediate cell population, the method comprising:
(a) culturing pluripotent stem cells in a mesoderm induction medium comprising activin A and a glycogen synthase kinase-3 inhibitor for a first period of time to produce a first intermediate cell population; and
(b) culturing the first intermediate cell population in an intermediate mesoderm induction medium comprising a retinoic acid pathway modulator (RAPM), FGF, and a glycogen synthase kinase-3 inhibitor for a second period of time, thereby producing the second intermediate cell population.

10. The method of any one of claims 1-4 and 9, wherein at least a portion of cells in the first intermediate cell population express Brachyury.

11. The method of any one of claims 1-4, 8, and 9, wherein at least a portion of the cells in the second intermediate cell population express OSR1, PAX2, or LHX

12. The method of any one of claims 1-6, wherein at least a portion of the cells in the gonadal cell population express FOXL2, NR2F2, or RUNX1

13. The method of any one of claims 1, 2, 7, and 9-11, wherein the pluripotent stem cells are seeded at a density of about 10,000 to about 40,000 cells per cm².

14. The method of any one of claims 1, 2, 7, and 9-13, wherein the pluripotent stem cells are seeded in a culture plate coated with fibronectin.

15. The method of any one of claims 1, 2, 7, and 9-13, wherein the pluripotent stem cells are seeded in a culture plate coated with matrigel.

16. The method of any one of claims 1-15, wherein the mesoderm induction medium further comprises FGF.

17. The method of any one of claims 1-16, wherein the mesoderm induction medium further comprises BMP4.

18. The method of any one of claims 1-17, wherein the mesoderm induction medium further comprises a glycogen synthase kinase-3 inhibitor.

19. The method of any one of claims 1-18, wherein the mesoderm induction medium further comprises an apoptosis inhibitor.

20. The method any one of claims 1-19, wherein the concentration of Activin A in the mesoderm induction medium is about 30 ng/mL to about 70 ng/mL.

21. The method of claim 20, wherein the concentration of Activin A in the mesoderm induction medium is about 50 ng/mL.

22. The method of any one of claims 16-21, wherein the FGF in the mesoderm induction medium is FGF2.

23. The method of claim 22, wherein the concentration of FGF2 in the mesoderm induction medium is about 5 ng/mL to about 20 ng/ml, optionally wherein the concentration of FGF2 in the mesoderm induction medium is about 12 ng/mL.

24. The method of any one of claims 17-23, wherein the concentration of BMP4 in the mesoderm induction medium is about 10 ng/mL to about 50 ng/mL, optionally wherein the concentration of BMP4 in the mesoderm induction medium is about 30 ng/ml.

25. The method any one of claims 18-24, wherein the glycogen synthase kinase-3 inhibitor in the mesoderm induction medium is CHIR99021.

26. The method of claim 25, wherein the concentration of CHIR99021 is about 1 μM to about 5 μM.

27. The method of claim 26, wherein the concentration of CHIR99021 in the mesoderm induction medium is about 3 μM.

28. The method of any one of claims 19-27, wherein within the mesoderm-induction medium:
(A) the apoptosis inhibitor is Y-27632, optionally wherein the concentration of Y-27632 is about 5 μM to about 20 μM; or
(B) the apoptosis inhibitor comprises Chroman1, Emricasan, and Trans-ISRIB, optionally wherein the concentration of Chroman1 is about 30 nM to about 70 nM, the concentration of Emricasan is about 2 μM to about 10 M, and the concentration of Trans-ISRIB is about 0.2 μM to about 2 μM.

29. The method of claim 28, wherein within the mesoderm induction media:
(A) the concentration of Y-27632 is about 10 μM; or
(B) the concentration of Chroman1 is about 50 nM, the concentration of Emricasan is about 5 μM, and the concentration of Trans-ISRIB is about 0.7 μM.

30. The method of any one of claims 1-29, wherein the period of time for culturing in mesoderm induction medium is about 24 hours to about 96 hours.

31. The method of claim 30, wherein the period of time for culturing in mesoderm induction medium is about 24 hours to about 72 hours.

32. The method of claim 31, wherein the period of time for culturing in mesoderm induction medium is about 56 hours to about 72 hours

33. The method of claim any one of claims 1-32, wherein at least 90% of cells in the first intermediate cell population express Brachyury.

34. The method of claim any one of claims 1-33, wherein at least 80% of cells in the first intermediate cell population express one or more of: MIXL1, N-Cadherin, EpCam, NCAM.

35. The method of claim 1-34, wherein at least 90% of cells in the first intermediate cell population expresses: Brachyury, N-Cadherin, EpCam, and NCAM

36. The method of claim any one of claims 1-35, wherein at least 90% of cells in the first intermediate cell population are mesoderm or mesoderm-like cells.

37. The method of claim 36, wherein the first intermediate cell population consists essentially of mesoderm or mesoderm-like cells.

38. The method of any one of claims 1-37, wherein the first intermediate cell population is replated onto a new fibronectin-coated culture plate prior to culturing in intermediate mesoderm induction medium; optionally wherein the first intermediate cell population is enzymatically detached, centrifuged and resuspended before replating.

39. The method of claim 38, wherein the first intermediate cell population is plated a density of about 5,000 to about 25,000 cells/cm².

40. The method of any one of claims 1-39, wherein the intermediate mesoderm induction medium further comprises an FGF.

41. The method of any one of claims 1-40, wherein the intermediate mesoderm induction medium further comprises a glycogen synthase kinase-3 inhibitor.

42. The method of any one of claims 1-41, wherein the intermediate mesoderm induction medium further comprises Activin A.

43. The method of any one of claims 1-42, wherein the intermediate mesoderm induction medium further comprises an apoptosis inhibitor.

44. The method of claim 43, wherein the method comprises culturing the first intermediate cell population
(i) first in the intermediate mesoderm induction medium with a first concentration of the apoptosis inhibitor,
(ii) subsequently in the intermediate mesoderm induction medium with no more than a second concentration of the apoptosis inhibitor.

45. The method of any one of claims 1-44, wherein the RAPM in the intermediate mesoderm induction medium is an RAR agonist, optionally wherein the RAPM comprises retionic acid (RA) and/or TTNPB.

46. The method of any one of claims 1-45, wherein:
(c) the RAPM is RA, wherein the concentration of RA in the intermediate mesoderm induction medium is about 0.5 μM to about 2 μM; and/or
(d) the RAPM is TTNPB, wherein the concentration of TTNPB in the intermediate mesoderm induction medium is about 0.2 μM to about 1 μM.

47. The method of claim 46, wherein:
(c) the concentration of RA in the intermediate mesoderm induction medium is about 1 μM; and/or
(d) the concentration of TTNPB in the intermediate mesoderm induction medium is about 0.5 μM.

48. The method of any one of claims 40-47, wherein the FGF in the intermediate mesoderm induction medium is FGF2, wherein the concentration of the FGF2 is about 10 ng/ml to about 30 ng/mL.

49. The method of claim 48, wherein the concentration of the FGF2 in the intermediate mesoderm induction medium is about 20 ng/mL.

50. The method of any one of claims 41-49, wherein the glycogen synthase kinase-3 inhibitor in the intermediate mesoderm induction medium is CHIR99021, wherein the concentration of CHIR99021 is about 1 μM to about 5 μM.

51. The method of claim 50, wherein the concentration of CHIR99021 in the intermediate mesoderm induction medium is about 2 μM or about 3 μM.

52. The method of any one of claims 42-51, wherein the concentration of Activin A in the intermediate mesoderm induction medium is about 10 ng/mL to about 50 ng/mL.

53. The method of claim 52, wherein the concentration of Activin A in the intermediate mesoderm induction medium is about 30 ng/mL

54. The method of any one of claims 43 and 46-53, wherein within the mesoderm-induction medium:
(A) the apoptosis inhibitor is Y-27632, wherein the concentration of Y-27632 is about 5 μM to about 20 μM; or
(B) the apoptosis inhibitor comprises Chroman1, Emricasan, and Trans-ISRIB, wherein the concentration of Chroman1 is about 30 nM to about 70 nM, the concentration of Emricasan is about 2 μM to about 10 μM, and the concentration of Trans-ISRIB is about 0.2 μM to about 2 μM.

55. The method of any one of claims 45-54, wherein the apoptosis inhibitor in the intermediate mesoderm induction medium is Y-27632, wherein the method comprises culturing the first intermediate cell population:
(i) first in the intermediate mesoderm induction medium comprising about 10 μM of Y-27632; and
(ii) subsequently in the intermediate mesoderm induction medium with no more than about 2 μM of Y-27632.

56. The method of any one of claims 45-55, wherein the method comprises culturing the first intermediate cell population:
(i) first in the intermediate mesoderm induction medium comprising about 10 μM of Y-27632 for about 24 hours,
(ii) subsequently in the intermediate mesoderm induction medium with no more than about 2 μM of Y-27632 for about 5-6 days.

57. The method of any one of claims 1-56, wherein the period of time for culturing in the intermediate mesoderm induction medium is about 4-14 days.

58. The method of any one of claims 1-57, wherein the period of time for culturing in the intermediate mesoderm induction medium is about 5-9 days.

59. The method of any one of claim 1-58, wherein the apoptosis inhibitor in the intermediate mesoderm induction medium is Y-27632, wherein the concentration of Y-27632 is:
(a) about 10 μM during the first 24 hours;
(b) no more than about 2 μM between 24 hours and 72 hours;
(c) no more than about 0.5 μM between 72 hours and 120 hours; or
(d) no more than about 0.1 μM after 120 hours,
during the culturing in the intermediate mesoderm induction medium.

60. The method of any one of claims 44-59, wherein the first intermediate cell population is cultured in intermediate mesoderm induction medium comprising an apoptosis inhibitor for about 24 hours, and wherein after about 24 hours, a portion of the medium is first replaced with an intermediate mesoderm induction medium not comprising the apoptosis inhibitor.

61. The method of claim 60, wherein portion of the medium is about 80% of the medium.

62. The method of claim 60 or 61, wherein the method further comprises subsequent medium replacements, wherein the medium replacements comprise replacing a portion of the medium every about 48 hours after the initial 24 hours of culturing.

63. The method of claim 62, wherein the portion of the medium in each subsequent medium replacement is about 80% of the medium.

64. The method of any one of claims 1-63, wherein at least 90% of cells in the second intermediate cell population expresses one or more of: OSR1, PAX2, LHX1, and RUNX1.

65. The method of any one of claims 1-64, wherein at least 90% of cells in the second intermediate cell population expresses two or more of: OSR1, PAX2, LHX1, and RUNX1

66. The method of any one of claims 1-65, wherein at least 90% of the cells in the second intermediate cell population expresses OSR1, PAX2, and LHX1.

67. The method of any one of claims 1-66, wherein at least 90% of cells in the second intermediate cell population are intermediate mesoderm or intermediate mesoderm-like cells.

68. The method of claim 67, wherein the second intermediate cell population consists essentially of intermediate mesoderm or intermediate mesoderm-like cells.

69. The method of any one of claims 1-68, wherein the second intermediate cell population is replated onto a new fibronectin-coated culture plate prior to culturing in gonadal induction medium; optionally wherein the second intermediate cell population is enzymatically detached, centrifuged and resuspended before replating.

70. The method of claim 69, wherein the second intermediate cell population is plated at a density of about 5,000 to about 25,000 cells/cm².

71. The method of any one of claims 1-70, wherein the gonadal induction medium further comprises an RAPM.

72. The method of any one of claims 1-71, wherein the gonadal induction medium further comprises an apoptosis inhibitor.

73. The method of any one of claims 1, 3, 5 and 10-72, wherein the gonadal induction medium further comprises follistatin.

74. The method of any one of claims 1-73, wherein the concentration of follistatin in the gonadal induction medium is about 10 ng/mL to about 50 ng/ml.

75. The method of claim 74, wherein the concentration of follistatin in the gonadal induction medium about 25 ng/ml.

76. The method of any one of claims 1, 3, 5 and 10-75, wherein the BMP in the gonadal induction medium comprises BMP4, BMP2, BMP7, BMP15, or any combinations thereof.

77. The method of any one of claims 1, 3, 5 and 10-76, wherein the total concentration of BMP in the gonadal induction medium is about 5 ng/mL to about 20 ng/ml, or about 20 ng/ml to about 70 ng/mL.

78. The method of claim 77, wherein the total concentration of BMP in the gonadal induction medium is about 10 ng/ml or about 50 ng/ml.

79. The method of any one of claims 1-78, wherein the concentration of BMP4 in the gonadal induction medium is about 5 ng/ml to about 20 ng/ml, or about 20 ng/ml to about 70 ng/mL.

80. The method of claim 79, wherein the concentration of BMP4 in the gonadal induction medium is about 10 ng/ml or about 50 ng/mL.

81. The method of any one of claims 1-80, wherein the FGF in the gonadal induction medium comprises FGF2, FGF9, FGF10, FGF16, FGF17, FGF18, FGF19, or any combinations thereof.

82. The method of any one of claims 1-81, wherein the concentration of FGF is about 1 ng/mL to about 10 ng/ml or about 5 ng/ml to about 25 ng/ml.

83. The method of claim 82, wherein the concentration of FGF is about 5 ng/mL or about 10 ng/ml

84. The method of any one of claims 1-83, wherein the FGF in the gonadal induction medium is FGF2, wherein the concentration of the FGF2 is about 1 ng/mL to about 10 ng/mL or about 5 ng/mL to about 25 ng/mL.

85. The method of claim 84, wherein the concentration of the FGF2 in the gonadal induction medium is about 5 ng/ml or about 10 ng/mL.

86. The method of any one of claims 71-85, wherein the RAPM in the gonadal induction medium is an RAR agonist, optionally wherein the RAPM comprises retinoic acid (RA) and/or TTNPB.

87. The method of claim 86, wherein:
(c) the RAPM is RA, wherein the concentration of RA in the gonadal induction medium is about 0.5 μM to about 2 μM; and/or
(d) the RAPM is TTNPB, wherein the concentration of TTNPB in the gonadal induction medium is about 0.2 μM to about 1 μM.

88. The method of any one of claims 72-87, wherein within the gonadal induction medium:
(A) the apoptosis inhibitor is Y-27632, optionally wherein the concentration of Y-27632 is about 5 μM to about 20 μM; or
(B) the apoptosis inhibitor comprises Chroman1, Emricasan, and Trans-ISRIB, optionally wherein the concentration of Chroman1 is about 30 nM to about 70 nM, the concentration of Emricasan is about 2 μM to about 10 μM, and the concentration of Trans-ISRIB is about 0.2 μM to about 2 μM.

89. The method of claim 88, wherein within the gonadal induction medium:
(A) the concentration of Y-27632 is about 10 μM; or
(B) the concentration of Chroman1 is about 50 nM, the concentration of Emricasan is about 5 μM, and the concentration of Trans-ISRIB is about 0.7 μM.

90. The method of any one of claims 1-89, wherein the period of time for culturing in gonadal induction medium is about 5 days to about 21 days.

91. The method of claim 90, wherein the period of time for culturing in gonadal induction medium is about 7 days to about 14 days.

92. The method of any one of claims 1-91, wherein the gonadal cell population comprises ovarian somatic cells.

93. The method of any one of claims 1-92, wherein the gonadal cell population consists of ovarian somatic cells.

94. The method of any one of claims 1-93, wherein at least 20% of cells within the gonadal cell population are FOXL2-positive cells.

95. The method of any one of claims 1-94, wherein at least 20% of the cells within the gonadal cell population are NR2F2-positive cells.

96. The method of any one of claims 1-95, wherein at least 20% of the cells within the gonadal cell population are KRT19-positive cells

97. The method of any one of claims 1-96, wherein at least 90% of the gonadal cell population are FOXL2-positive cells, NR2F2-positive cells, and/or KRT-19 positive cells.

98. The method of claim 97, wherein the gonadal cell population consists essentially of FOXL2-positive cells, NR2F2-positive cells, and/or KRT-19 positive cells.

99. The method of claim 97 or 98, wherein the FOXL2-positive cells comprise granulosa cells; wherein the NR2F2-positive cells comprise stroma cells and/or granulosa cells; and wherein the KRT-19 positive cells comprise ovarian epithelial cells.

100. The method of any one of claims 1-99, wherein:
(I) the amount of NR2F2-expressing cells in the gonadal cell population is increased; and/or
(II) the amount of FOXL2-expressing cells in the gonadal cell population is increased; and/or
(III) the amount of RUNX1-expressing cells in the gonadal cell population is increased; and/or
(IV) the amount of WNT6-expressing cells in the gonadal cell population is increased; and/or
(V) the amount of NR5A1-expressing cells in the gonadal cell population is increased; and/or
(VI) the amount of OSR1-expressing cells in the gonadal cell population is increased; and/or
(VII) the amount of LHX9-expressing cells in the gonadal cell population is increased; and/or
(VIII) the amount of EMX2-expressing cells in the gonadal cell population is increased,
as compared to a corresponding gonadal cell population generated by a method wherein the intermediate mesoderm induction medium does not comprise RAPM.

101. The method of any one of claims 1-99, wherein:
(I) the amount of NR2F2-expressing cells in the gonadal cell population is increased; and/or
(II) the amount of FOXL2-expressing cells in the gonadal cell population is increased; and/or
(III) the amount of RUNX1-expressing cells in the gonadal cell population is increased; and/or
(IV) the amount of WNT6-expressing cells in the gonadal cell population is increased; and/or
(V) the amount of NR5A1-expressing cells in the gonadal cell population is increased; and/or
(VI) the amount of OSR1-expressing cells in the gonadal cell population is increased,
as compared to a corresponding gonadal cell population generated by a method wherein the intermediate mesoderm induction medium comprises a lower concentration of RAPM.

102. The method of any one of claims 1-99, wherein:
(I) the amount of NR2F2-expressing cells in the gonadal cell population is increased; and/or
(II) the amount of FOXL2-expressing cells in the gonadal cell population is increased; and/or
(III) the amount of RUNX1-expressing cells in the gonadal cell population is increased; and/or
(IV) the amount of WNT6-expressing cells in the gonadal cell population is increased; and/or
(V) the amount of NR5A1-expressing cells in the gonadal cell population is increased; and/or
(VI) the amount of OSR1-expressing cells in the gonadal cell population is increased; and/or
(VII) the amount of LHX9-expressing cells in the gonadal cell population is increased; and/or
(VIII) the amount of EMX2-expressing cells in the gonadal cell population is increased,
as compared to a corresponding gonadal cell population generated by a method wherein the intermediate mesoderm induction medium does not comprise glycogen synthase kinase-3 inhibitor.

103. The method of any one of claims 1-99, wherein:
(I) the amount of NR2F2-expressing cells in the gonadal cell population is increased; and/or
(II) the amount of FOXL2-expressing cells in the gonadal cell population is increased; and/or
(III) the amount of RUNX1-expressing cells in the gonadal cell population is increased; and/or
(IV) the amount of WNT6-expressing cells in the gonadal cell population is increased; and/or
(V) the amount of NR5A1-expressing cells in the gonadal cell population is increased; and/or
(VI) the amount of OSR1-expressing cells in the gonadal cell population is increased; and/or
(VII) the amount of LHX9-expressing cells in the gonadal cell population is increased; and/or
(VIII) the amount of EMX2-expressing cells in the gonadal cell population is increased,
as compared to a corresponding gonadal cell population generated by a method wherein the intermediate mesoderm induction medium comprises a lower concentration of glycogen synthase kinase-3 inhibitor.

104. The method of any one of claims 1-99, wherein:
(I) the amount of LHX1 expression in the second intermediate cell population is increased; and/or
(II) the amount of PAX2 expression in the second intermediate cell population is increased; and/or
(III) the amount of WT1 expression in the second intermediate population is increased; and/or
(IV) the amount of RUNX1 expression in the second intermediate cell population is increased; and/or
(V) the viability of the second intermediate cell population is increased; and/or
(VI) the cell morphology of the second intermediate cell population is more uniform,
as compared to a corresponding second intermediate cell population generated by a method wherein the intermediate mesoderm induction medium does not comprise RAPM.

105. The method of any one of claims 1-99, wherein:
(I) the amount of LHX1 expression in the second intermediate cell population is increased; and/or
(II) the amount of PAX2 expression in the second intermediate cell population is increased; and/or
(III) the amount of WT1 expression in the second intermediate population is increased; and/or
(IV) the amount of RUNX1 expression in the second intermediate cell population is increased; and/or
(V) the viability of the second intermediate cell population is increased; and/or
(VI) the cell morphology of the second intermediate cell population is more uniform,
as compared to a corresponding second intermediate cell population generated by a method wherein the intermediate mesoderm induction medium comprises a lower concentration of RAPM

106. The method of any one of claims 1-99, wherein:
(I) the amount of LHX1 expression in the second intermediate cell population is increased; and/or
(II) the amount of PAX2 expression in the second intermediate cell population is increased; and/or
(III) the amount of WT1 expression in the second intermediate population is increased; and/or
(IV) the amount of RUNX1 expression in the second intermediate cell population is increased; and/or
(V) the viability of the second intermediate cell population is increased; and/or
(VI) the cell morphology of the second intermediate cell population is more uniform,
as compared to a corresponding second intermediate cell population generated by a method wherein the intermediate mesoderm induction medium does not comprise glycogen synthase kinase-3 inhibitor.

107. The method of any one of claims 1-99, wherein:
(I) the amount of LHX1 expression in the second intermediate cell population is increased; and/or
(II) the amount of PAX2 expression in the second intermediate cell population is increased; and/or
(III) the amount of WT1 expression in the second intermediate population is increased; and/or
(IV) the amount of RUNX1 expression in the second intermediate cell population is increased; and/or
(V) the viability of the second intermediate cell population is increased; and/or
(VI) the cell morphology of the second intermediate cell population is more uniform,
as compared to a corresponding second intermediate cell population generated by a method wherein the intermediate mesoderm induction medium comprises a lower concentration of glycogen synthase kinase-3 inhibitor.

108. The method of any one of claims 1-99, wherein:
(I) the potential of the second intermediate cell population to differentiate into NR2F2-expressing gonadal cells is increased; and/or
(II) the potential of the second intermediate cell population to differentiate into FOXL2-expressing gonadal cells is increased; and/or
(III) the potential of the second intermediate cell population to differentiate into RUNX1-expressing gonadal cells is increased; and/or
(IV) the potential of the second intermediate cell population to differentiate into WNT6-expressing gonadal cells is increased; and/or
(V) the potential of the second intermediate cell population to differentiate into NR5A1-expressing gonadal cells is increased; and/or
(VI) the potential of the second intermediate cell population to differentiate into OSR1-expressing gonadal cells is increased,
as compared to a corresponding second intermediate cell population generated by a method wherein the intermediate mesoderm induction medium does not comprise RAPM.

109. The method of any one of claims 1-99, wherein:
(I) the potential of the second intermediate cell population to differentiate into NR2F2-expressing gonadal cells is increased; and/or
(II) the potential of the second intermediate cell population to differentiate into FOXL2-expressing gonadal cells is increased; and/or
(III) the potential of the second intermediate cell population to differentiate into RUNX1-expressing gonadal cells is increased; and/or
(IV) the potential of the second intermediate cell population to differentiate into WNT6-expressing gonadal cells is increased; and/or
(V) the potential of the second intermediate cell population to differentiate into NR5A1-expressing gonadal cells is increased; and/or
(VI) the potential of the second intermediate cell population to differentiate into OSR1-expressing gonadal cells is increased,
as compared to a corresponding second intermediate cell population generated by a method wherein the intermediate mesoderm induction medium comprises a lower concentration of RAPM

110. The method of any one of claims 1-99, wherein:
(I) the potential of the second intermediate cell population to differentiate into NR2F2-expressing gonadal cells is increased; and/or
(II) the potential of the second intermediate cell population to differentiate into FOXL2-expressing gonadal cells is increased; and/or
(III) the potential of the second intermediate cell population to differentiate into RUNX1-expressing gonadal cells is increased; and/or
(IV) the potential of the second intermediate cell population to differentiate into WNT6-expressing gonadal cells is increased; and/or
(V) the potential of the second intermediate cell population to differentiate into NR5A1-expressing gonadal cells is increased; and/or
(VI) the potential of the second intermediate cell population to differentiate into OSR1-expressing gonadal cells is increased,
as compared to a corresponding second intermediate cell population generated by a method wherein the intermediate mesoderm induction medium does not comprise glycogen synthase kinase-3 inhibitor.

111. The method of any one of claims 1-99, wherein:
(I) the potential of the second intermediate cell population to differentiate into NR2F2-expressing gonadal cells is increased; and/or
(II) the potential of the second intermediate cell population to differentiate into FOXL2-expressing gonadal cells is increased; and/or
(III) the potential of the second intermediate cell population to differentiate into RUNX1-expressing gonadal cells is increased; and/or
(IV) the potential of the second intermediate cell population to differentiate into WNT6-expressing gonadal cells is increased; and/or
(V) the potential of the second intermediate cell population to differentiate into NR5A1-expressing gonadal cells is increased; and/or
(VI) the potential of the second intermediate cell population to differentiate into OSR1-expressing gonadal cells is increased,
as compared to a corresponding second intermediate cell population generated by a method wherein the intermediate mesoderm induction medium comprises a lower concentration of glycogen synthase kinase-3 inhibitor.

112. The method of any one of claims 1-111, wherein:
(I) the amount of FOXL2 expression in the gonadal cell population is decreased; and/or
(II) the amount of expression of NR1H4 and/or KITLG in the gonadal cell population is decreased; and/or
(III) the amount of KRT-19 expression in the gonadal cell population is increased; and/or
(IV) the amount of cytoplasmic KRT-19 expression in the gonadal cell population is increased; and/or
(V) the amount of expression of MSLN, LRRN4, and/or TMEM151A in the gonadal cell population is increased,
as compared to a corresponding gonadal cell population generated by a method wherein the gonadal induction medium comprises a lower concentration of RAPM.

113. The method of any one of claims 1-111, wherein:
(I) the amount of FOXL2 expression in the gonadal cell population is decreased; and/or
(II) the amount of expression of NR1H4 and/or KITLG in the gonadal cell population is decreased; and/or
(III) the amount of KRT-19 expression in the gonadal cell population is increased; and/or
(IV) the amount of cytoplasmic KRT-19 expression in the gonadal cell population is increased; and/or
(V) the amount of expression of MSLN, LRRN4, and/or TMEM151A in the gonadal cell population is increased,
as compared to a corresponding gonadal cell population generated by a method wherein the gonadal induction medium does not comprise a RAPM.

114. The method of any one of claims 1-111, wherein:
(I) the amount of FOXL2 expression in the gonadal cell population is decreased; and/or
(II) the amount of expression of NR1H4 and/or KITLG in the gonadal cell population is decreased; and/or
(III) the amount of KRT-19 expression in the gonadal cell population is increased; and/or
(IV) the amount of cytoplasmic KRT-19 expression in the gonadal cell population is increased; and/or
(V) the amount of expression of MSLN, LRRN4, and/or TMEM151A in the gonadal cell population is increased,
as compared to a corresponding gonadal cell population generated by a gonadal induction step comprising contacting with an RAPM for a shorter period of time.

115. The method of any one of claims 1-114, wherein the pluripotent stem cells are mammalian stem cells.

116. The method of any one of claims 1-115, wherein the pluripotent stem cells are human pluripotent stem cells.

117. The method of any one of claims 1-115, wherein the pluripotent stem cells are bovine stem cells.

118. The method of any one of claims 1-115, wherein the pluripotent stem cells are murine pluripotent stem cells.

119. The method of any one of claims 115-118, wherein the pluripotent stem cells are embryonic stem cells or induced pluripotent stem cells.

120. The method of any one of claims 1-6 and 10-119, wherein the gonadal population comprises one or more populations selected from granulosa cells, ovarian stroma cells and epithelial cells or a combination thereof.

121. The method of any one of claims 1-6 and 10-120, wherein the gonadal population comprises a mixture of granulosa cells, ovarian stroma cells and epithelial cells

122. A gonadal cell population produced by the method of any one of claims 1-6 and 10-121.

123. The gonadal cell population of claim 122, wherein the gonadal population comprises one or more populations selected from granulosa cells, ovarian stroma cells and epithelial cells or a combination thereof.

124. The gonadal cell population of claim 122 or claim 123, wherein the gonadal population comprises a mixture of granulosa cells, ovarian stroma cells and epithelial cells

125. A first intermediate cell population produced by the method of any one of claims 7 and 13-119.

126. A second intermediate cell population produced by the method of any one of claims 6-11 and 13-119.

127. A method of producing a granulosa cell, the method comprising:
culturing a pluripotent stem cell in the presence of activin A, a glycogen synthase kinase-3 inhibitor, and a ROCK inhibitor to produce an incipient mesoderm-like cell (iMeLC),
culturing the iMeLC in the presence of FGF2, a glycogen synthase kinase-3 inhibitor, and a ROCK inhibitor for a first period of time,
reducing an amount of the ROCK inhibitor in the iMeLC culture and then culturing the iMeLC for a second period of time to produce an intermediate mesoderm cell, and
culturing the intermediate mesoderm cell in the presence of follistatin, BMP4, FGF2, and a ROCK inhibitor to produce the granulosa cell.

128. The method of claim 127, wherein the glycogen synthase kinase-3 inhibitor is CHIR99021.

129. The method of claim 127 or 128, wherein the ROCK inhibitor is Y-27632 or CET.

130. The method of any one of claims 127-129, wherein the pluripotent stem cell is cultured for about 56 to 72 hours.

131. The method of any one of claims 127-130, wherein the pluripotent stem cell is cultured for about 65 hours.

132. The method of any one of claims 127-131, wherein pluripotent stem cell is cultured in a medium comprising the activin A, glycogen synthase kinase-3 inhibitor, and ROCK inhibitor, and wherein the medium is replaced with fresh medium every about 24 hours.

133. The method of any one of claims 127-132, wherein the first period of time is about 24 hours.

134. The method of any one of claims 127-133, wherein the second period of time is about 5 or 6 days.

135. The method of any one of claims 127-134, wherein the iMeLC is cultured in a medium comprising the FGF2, Glycogen synthase kinase-3 inhibitor, and ROCK inhibitor for the first period of time, and wherein after the first period of time a portion of the medium is replaced with a medium comprising FGF2 and glycogen synthase kinase-3 inhibitor but not a ROCK inhibitor.

136. The method of claim 135, wherein the portion of the medium is about 80% of the medium.

137. The method of claim 135 or 136, wherein the method further comprises replacing a second portion of the medium every about 48 hours during the second time period.

138. The method of claim 137, wherein the second portion of the medium is about 80% of the medium.

139. The method of any one of claims 127-138, wherein the intermediate mesoderm cell is cultured for a period of about 5-7 days.

140. The method of any one of claims 127-139, wherein the intermediate mesoderm cell is cultured in a medium comprising the follistatin, BMP4, FGF2, and ROCK inhibitor, and wherein a portion of the medium is replaced with fresh medium every about 24 to 48 hours.

141. The method of any one of claims 127-140, wherein the iMeLC expresses Brachyury.

142. The method of any one of claims 127-139, wherein the intermediate mesoderm cell expresses OSR1, PAX2, and LHX1.

143. The method of any one of claims 127-142, wherein the granulosa cell expresses FOXL2 and CONNEXIN43.

144. The method of any one of claims 127-143, wherein the pluripotent stem cell is a human induced pluripotent stem cell.

145. A population comprising granulosa cells produced by the method of any one of claims 127-144.

146. The method or population of any one of claims 1-145, wherein:
(a) one or more of the culturing steps comprises adherent culture; and/or
(b) one or more of the culturing steps comprises 3-dimensional organoid culture.

147. The method or population of any one of claims 1-146, wherein one or more of the culturing steps comprises adherent culture.

148. The method or population of any one of claims 1-147, wherein one or more of the culturing step comprises 3-dimensional organoid culture.

149. An in vitro stem cell-derived gonadal somatic cell population comprising FOXL2-expressing cells, NR2F2-expressing cells and/or KRT-19 expressing cells, optionally wherein the gonadal somatic cell population is an ovarian somatic cell population.

150. The gonadal somatic cell population of claim 149, wherein the population comprises at least a first cell type expressing FOXL2, a second cell type expressing NR2F2, and a third cell type expressing KRT-19.

151. The gonadal somatic cell population of claim 149 or 150, wherein:
(a) at least 20% of cells within the cell population are FOXL2-positive cells; and/or
(b) at least 20% of the cells within the cell population are NR2F2-positive cells; and/or
(c) at least 20% of the cells within the gonadal cell population are KRT19-positive cells.

152. The gonadal somatic cell population of any one of claims 149-151, wherein at least 90% of the gonadal cell population are FOXL2-positive cells, NR2F2-positive cells, and/or KRT-19 positive cells; optionally wherein:
the gonadal somatic cell population consists essentially of FOXL2-positive cells, NR2F2-positive cells, and/or KRT-19 positive cells.

153. The gonadal somatic cell population of claim 151 or 152, wherein the FOXL2-positive cells comprise granulosa cells; wherein the NR2F2-positive cells comprise ovarian stromal cells and/or granulosa cells; and wherein the KRT-19 positive cells comprise ovarian epithelial cells.

154. The gonadal somatic cell population of any one of claims 149-153, wherein the cell population is derived in a process comprising:
(a) culturing pluripotent stem cells in a mesoderm induction medium comprising Activin A for a first period of time to produce a first intermediate cell population;
(b) culturing the first intermediate cell population in an intermediate mesoderm induction medium comprising an retinoic acid pathway modulator (RAPM) for a second period of time, thereby producing a second intermediate cell population; and
(c) culturing the second intermediate cell population in a gonadal induction medium comprising BMP and FGF, and optionally follistatin, for a third period of time to produce the gonadal cell population.
